(19) United States
(12) Patent Application Publication
(10) Pub. No.: US 2026/0078111 A1
(43) Pub. Date: Mar. 19, 2026

(54) CARBONIC ANHYDRASE ENZYME INHIBITORS AND METHODS OF USE THEREOF

(71) Applicants: RUTGERS, THE STATE UNIVERSITY OF NEW JERSEY, New Brunswick, NJ (US); NEMAGEN DISCOVERIES, INC., Princeton, NJ (US)

(72) Inventors: Mark C. SIRACUSA, New Brunswick, NJ (US); Jacques Y. ROBERGE, New Brunswick, NJ (US); John A. GILLERAN, New Brunswick, NJ (US); John PIWINSKI, Princeton, NJ (US)

(73) Assignees: RUTGERS, THE STATE UNIVERSITY OF NEW JERSEY, New Brunswick, NJ (US); NEMAGEN DISCOVERIES, INC., Princeton, NJ (US)

(21) Appl. No.: 19/130,641

(22) PCT Filed: Dec. 7, 2023
(86) PCT No.: PCT/US2023/082927

§ 371 Date: May 16, 2025

Related U.S. Application Data
(60) Provisional application No. 63/430,904, filed on Dec. 7, 2022.

(51) Int. Cl. C07D 413/10; A61K 31/404; A61K 31/4178; A61K 31/4439; A61K 31/454; A61K 31/4709; A61K 31/4725; A61K 31/496; A61K 31/5377; A61P 37/08; C07D 209/34; C07D 401/04; C07D 401/06; C07D 401/10; C07D 403/10; C07D 405/04; C07D 405/06

(52) U.S. Cl. CPC C07D 413/10; A61K 31/404; A61K 31/4178; A61K 31/4439; A61K 31/454; A61K 31/4709; A61K 31/4725; A61K 31/496; A61K 31/5377; A61P 37/08; C07D 209/34; C07D 401/04; C07D 401/06; C07D 401/10; C07D 403/10; C07D 405/04; C07D 405/06

(57) ABSTRACT

Provided herein are novel compounds, such as a compound of Formula I or Formula A: or a salt thereof, wherein R¹-R³, L¹, R¹⁰, ring A and ring B have any of the values described in the specification, as well as compositions comprising the novel compounds herein. The compounds are typically carbonic anhydrase inhibitors and are useful for the prophylactic or therapeutic treatment of a disease or condition mediated by a carbonic anhydrase enzyme.

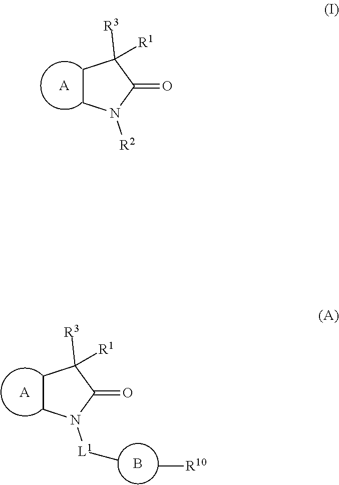

### PRIORITY

This application claims priority to U.S. Provisional Patent Application No. 63/430,904, which was filed on 7 Dec. 2022. The entire content of this United States Provisional patent application is hereby incorporated herein by reference.

### GOVERNMENT FUNDING

This invention was made with government support under AI123224 awarded by the National Institutes of Health. The government has certain rights in the invention.

### BACKGROUND

Mast cell-related diseases, such as allergies/allergic inflammation, mastocytosis, mast cell activation syndrome and anaphylaxis, are increasingly common. For example, allergic diseases have risen to epidemic proportions in developed areas of the world, and it is now estimated that 30-40% of the global population suffer from one or more allergic diseases, including atopic dermatitis, asthma and food allergies (Pawankar, et al., WAO White Book on Allergy 2011-2012: Executive Summary. World Allergy Organization (2012)). There are limited treatment options for patients suffering from chronic allergic inflammation and many of them have significant side effects (Bauer, et al., J Allergy Clin Immunol 135, 312-323 (2015)). For example, allergies and asthma are most commonly treated with steroids; however, it is well known that steroids have a limited effect in treating these disease states. Further, diseases such as mastocytosis and mast cell activation syndrome (MCAS) have very few effective treatments. Therefore, a compound that can effectively inhibit mast cell development and mast cell-mediated inflammation would fill an important clinical need.

While the cellular pathways and growth factors that regulate mast cell responses have been challenging to detect, a unique mast cell progenitor cell defined by its expression of the enzyme Carbonic anhydrase (Car) 1, was recently identified (Henry, E. K., et al., J Exp Med, 2016. 213(9): p. 1663-73; Inclan-Rico, J. M., et al., PLoS Pathogens, 2020. 16(5): p. e1008579; Voehringer, D., Nat Rev Immunol, 2013. 13(5): p. 362-75; Dahlin, J. S. and J. Hallgren, Mol Immunol, 2015. 63(1): p. 9-17). It was demonstrated that genetically or pharmacologically targeting Car1 via CRIPS/Cas9 technology or the use of Car enzyme inhibitors was sufficient to prevent murine mast cell responses in vitro. Further, targeting Car1 via Car enzyme inhibitors was also sufficient to prevent human mast cell development in culture assays (Henry, E. K., et al., J Exp Med, 2016. 213(9): p. 1663-73). Moreover, it was shown that targeting Car1 with Car enzyme inhibitors was sufficient to prevent murine mast cell responses and mast cell-mediated inflammation in vivo (Id.). Collectively, these data suggest that Car1 may be a therapeutic target for treating mast cell-related diseases.

There are several FDA-approved Carbonic anhydrase inhibitors that are actively used in clinics. Among these inhibitors is methazolamide (MZ). MZ is capable of inhibiting mast cell development and mast cell mediated-inflammation at a high dose. There is a need for new carbonic anhydrase inhibitors, such as those with improved potency and/or selectivity (e.g., for use in the treatment of allergic inflammation and other mast cell-associated disorders).

### SUMMARY

In various embodiments, the invention provides novel compounds that are carbonic anhydrase inhibitors, such as carbonic anhydrase 1 inhibitors, which are useful for treating diseases or conditions mediated by carbonic anhydrase, such as mastocytosis, mast cell-mediated inflammation, allergic diseases, bacterial infections, fungal infections and viral infections.

In one embodiment, the invention provides a compound of Formula I:

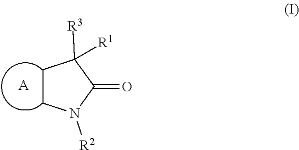

or a pharmaceutically acceptable salt thereof, wherein:
R¹ is aryl, 5-membered heteroaryl, 6-membered heteroaryl, or (C₁-C₃)alkyl that is substituted with aryl, 5-membered heteroaryl, or 6-membered heteroaryl, wherein any aryl, 5-membered heteroaryl, and 6-membered heteroaryl, is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;
R² is H, aryl, 5-membered heteroaryl, 6-membered heteroaryl, or (C₁-C₃)alkyl that is optionally substituted with aryl, 5-membered heteroaryl, or 6-membered heteroaryl, wherein any aryl, 5-membered heteroaryl, and 6-membered heteroaryl, is optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{c}R^{d}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;
R³ is H, fluoro, hydroxy, (C₁-C₆)alkyl, or (C₁-C₆)alkoxy, wherein any (C₁-C₆)alkyl and (C₁-C₆)alkoxy, is optionally substituted with one or more fluoro;
ring A is phenyl, 5-membered heteroaryl, or 6-membered heteroaryl, and ring A is optionally substituted with 1, 2, 3, or 4 groups, as valency permits, independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;
each R^{a} and R^{b} is independently selected from the group consisting of H, (C₁-C₆)alkyl, (C₃-C₆)cycloalkyl, and (C₃-C₆)cycloalkyl(C₁-C₆)alkyl; or R^{a} and R^{b} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl;
each R^{c} and R^{d} is independently selected from the group consisting of H, (C₁-C₆)alkyl, (C₃-C₆)cycloalkyl, and (C₃-C₆)cycloalkyl(C₁-C₆)alkyl; or R^{c} and R^{d} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl; and
each R^{e} and R^{f} is independently selected from the group consisting of H, (C₁-C₆)alkyl, (C₃-C₆)cycloalkyl, and (C₃-C₆)cycloalkyl(C₁-C₆)alkyl; or R^{e} and R^{f} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl. In some embodiments, the compound of Formula I can be characterized as having a structure according to a subformula of Formula I, such as Formula Ia, Ib, IIa, IIb, IIIa, or IIIb, as defined herein.

In some embodiments, the invention provides a compound of Formula A, or a pharmaceutically acceptable salt thereof:

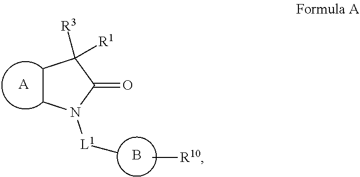

wherein:
R¹, R³, and ring A are as defined herein in connection with Formula I,
Ring B is absent, an optionally substituted carbocyclylene, optionally substituted arylene, optionally substituted heteroarylene, or optionally substituted heterocyclylene;
L¹ is absent or an optionally substituted C₁₋₃ alkylene; and
R¹⁰ is hydrogen, halo, hydroxy, cyano, nitro, NH₂, COOH, CONH₂, S(O)₂NH₂, G¹, OG¹, NHG¹, NG¹G¹, C(O)G¹, C(O)OG¹, C(O)NHG¹, C(O)NG¹G¹, S(O)₂G¹, S(O)₂NHG¹, or S(O)₂NG¹G¹, wherein G¹ at each occurrence is independently an optionally substituted (C₁-C₆)alkyl, optionally substituted (C₁-C₆)heteroalkyl, optionally substituted 3-7 membered carbocyclic, optionally substituted aryl, optionally substituted heteroaryl, or optionally substituted heterocyclyl. In some embodiments, the compound of Formula A can be characterized as having a structure according to a subformula of Formula A, such as Formula A-1, A-1-A, A-2, A-2-A, A-2-B, A-2-C, A-3, A-E1, or A-E2, as defined herein.

In some embodiments, the invention also provides a pharmaceutical composition comprising a compound of Formula I or Formula A, e.g., any of the subformulae or specific compounds according to Formula I or Formula A as defined herein, or a pharmaceutically acceptable salt thereof, and a pharmaceutically acceptable excipient.

Certain embodiments provide a method of inhibiting a carbonic anhydrase enzyme in vitro or in vivo comprising contacting the carbonic anhydrase enzyme with an effective amount of a compound of Formula I or Formula A, e.g., any of the subformulae or specific compounds according to Formula I or Formula A as defined herein, or a pharmaceutically acceptable salt thereof.

Certain embodiments provide a method of treating a disease or condition mediated by a carbonic anhydrase enzyme in a mammal (e.g., a human), comprising administering a compound of Formula I or Formula A, e.g., any of the subformulae or specific compounds according to Formula I or Formula A as defined herein, or a pharmaceutically acceptable salt thereof, to the mammal.

Certain embodiments provide a compound of Formula I or Formula A, e.g., any of the subformulae or specific compounds according to Formula I or Formula A as defined herein, or a pharmaceutically acceptable salt thereof, for the prophylactic or therapeutic treatment of a disease or condition mediated by a carbonic anhydrase enzyme.

Certain embodiments provide the use of a compound of Formula I or Formula A, e.g., any of the subformulae or specific compounds according to Formula I or Formula A as defined herein, or a pharmaceutically acceptable salt thereof, to prepare a medicament for treating a disease or condition mediated by a carbonic anhydrase enzyme.

In some embodiments, the invention also provides a compound of Formula I or Formula A, e.g., any of the subformulae or specific compounds according to Formula I or Formula A as defined herein, or a pharmaceutically acceptable salt thereof, for use in medical therapy.

In some embodiments, the invention also provides a pharmaceutical composition comprising a compound of Formula I or Formula A, e.g., any of the subformulae or specific compounds according to Formula I or Formula A as defined herein, or a pharmaceutically acceptable salt thereof, for use in the treatment of of a disease or condition mediated by a carbonic anhydrase enzyme.

In some embodiments, the invention also provides processes and intermediates disclosed herein that are useful for preparing a compound of Formula I or Formula A, e.g., any of the subformulae or specific compounds according to Formula I or Formula A as defined herein, or a salt thereof.

It is to be understood that both the foregoing summary and the following detailed description are exemplary and explanatory only, and are not restrictive of the invention herein.

### BRIEF DESCRIPTION OF THE DRAWINGS

FIG. 1. Murine bone marrow cells were cultured with media or IL-3 (10 ng/mL) for 7 days. Cells were treated with either vehicle, MZ or 4-(3-hydroxy-1-(4-methoxybenzyl)-2-oxoindolin-3-yl)benzenesulfonamide (CAR0037) at the indicated doses and mast cell numbers were quantified. Student's t-test (**, p<0.01). Results are representative of at least 3 separate experiments.

FIG. 2. Mice were infected with T. spiralis and treated with either vehicle, MZ or 4-(3-hydroxy-1-(4-methoxybenzyl)-2-oxoindolin-3-yl)benzenesulfonamide (CAR0037) (i.p). On day 10 post-infection intestinal mast cell responses were evaluated by esterase staining and histological analysis. Student's t-test. Results are representative of at least 4 separate experiments.

FIG. 3. Murine bone marrow cells were cultured with media or IL-3 (10 ng/mL) for 7 days. Cells were treated with either MZ (126 uM), NEM-14A (14A), NEM-15A (15A), or NEM-23A (23A) at the indicated doses and the percentage of inhibition (POI) of mast cell numbers was measured.

### DETAILED DESCRIPTION

The present disclosure generally relates to novel carbonic anhydrase inhibitors, such as carbonic anhydrase 1 inhibitors. The present disclosure is based in part on the discovery that novel compounds of Formula I or Formula A as defined herein can inhibit carbonic anhydrase in vitro and/or in vivo and in some cases with similar or better efficacy than certain FDA-approved carbonic anhydrase inhibitor such as methazolamide. The compounds herein represent a novel class of carbonic anhydrase inhibitors, which are useful for treating various diseases or disorders herein, such as mastocytosis, mast cell-mediated inflammation, and various allergic diseases.

### Definitions

Unless defined otherwise, all technical and scientific terms used herein have the same meaning as commonly understood by one of ordinary skill in the art to which the disclosure pertains.

It is meant to be understood that proper valences are maintained for all moieties and combinations thereof.

It is also meant to be understood that a specific embodiment of a variable moiety herein can be the same or different as another specific embodiment having the same identifier.

The present disclosure encompasses all combinations of the aspects and/or embodiments of the disclosure herein. It is understood that any and all embodiments of the present disclosure may be taken in conjunction with any other embodiment or embodiments to describe additional embodiments. It is also to be understood that each individual element of the embodiments is meant to be combined with any and all other elements from any embodiment to describe an additional embodiment.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

When a range of values is listed, it is intended to encompass each value and sub-range within the range. For example “C₁₋₆” is intended to encompass, Cl, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocycles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g., tricyclic and tetracyclic carbocycles with up to about 20 carbon atoms). The rings of the multiple condensed ring system can be connected to each other via fused, spiro and bridged bonds when allowed by valency requirements. For example, multicyclic carbocycles can be connected to each other via a single carbon atom to form a spiro connection (e.g., spiropentane, spiro[4,5]decane, etc.), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc.). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl). The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heteroaryl” as used herein, unless specified to have a different ring size, refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). Ring sulfur and nitrogen atoms can optionally be oxidized and the nitrogen heteroatom can optionally be quaternized. Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, and thiadiazolyl. In heteroaryl groups that contain one or more nitrogen atoms, the point of attachment can be a carbon or nitrogen atom, as valency permits. Heteroaryl bicyclic ring systems can include one or more heteroatoms in one or both rings. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl).

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Preferably, the C₁₋₄ heteroalkyl (or C₁₋₄ heteroalkylene) herein contains 1 or 2 heteroatoms, such as one oxygen, one nitrogen, two oxygens, two nitrogens, or one oxygen and one nitrogen. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, together with recitations of specific heteroalkyl groups, such as alkoxy or —NR′R″ or the like, it will be understood that the terms heteroalkyl and alkoxy or —NR′R″ are not redundant or mutually exclusive. Rather, the specific heteroalkyl groups are recited to add clarity. Thus, the term “heteroalkyl” should not be interpreted herein as excluding specific heteroalkyl groups, such as alkoxy or —NR′R″ or the like.

“Heterocyclyl” or “heterocyclic” as used by itself or as part of another group, unless specified to have a different ring size, refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings.

As used herein, unless specified or otherwise contrary, a “ring structure”, “cyclic structure”, or simply “ring”, with a designated number of ring members, such as a “3-10 membered ring structure”, a “3-12 membered ring structure”, or a “5- or 6-membered ring”, should be understood as encompassing any ring structure (e.g., carbocyclic, heterocyclic, aryl, heteroaryl, etc.) having the designated number of ring members, which can be (1) monocyclic or polycyclic (as chemically feasible), such as a monocyclic ring or a bicyclic ring (including fused, spiro, and bridged bicyclic ring, and those ring systems where two monocyclic rings are connected through a single or double bond); (2) aromatic, partially unsaturated, or fully saturated; and in the case of a polycyclic structure, each ring can be independently aromatic, partially unsaturated, or fully saturated; and (3) contain no heteroatom or 1-4 heteroatoms; in the case of a polycyclic structure, each ring can independently have no ring heteroatom or 1-4 ring heteroatoms (e.g., O, N, S, etc.). When a ring is said to contain a ring sulfur or nitrogen atom, the sulfur or nitrogen atom can be optionally oxidized. One or more ring carbon atoms in a ring structure can be present as C(═O). A fully saturated ring refers to a ring in which none of the ring carbon and nitrogen (if present) atoms forms a double bond or triple bond with any other atom. The ring structure can be optionally substituted with one or more substituents described herein. The substituents of a ring structure herein can also have a cyclic structure, and in some cases, two substituents of a ring structure may be said to be joined to form a cyclic structure.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

The term “alkanoyl” as used herein refers to a group (alkyl)-C(═O)—, wherein the term alkyl has the meaning defined herein.

As used herein, the term “heteroatom” is meant to include oxygen (O), nitrogen (N), sulfur (S) and silicon (Si).

As commonly understood in the art, alkylene, alkenylene, alkynylene, heteroalkylene, carbocyclylene, heterocyclylene, arylene, and heteroarylene refer to the corresponding divalent radicals of alkyl, alkenyl, alkynyl, heteroalkyl, carbocyclyl, heterocyclyl, aryl, and heteroaryl groups, respectively.

An “optionally substituted” group, such as an optionally substituted alkyl, optionally substituted alkenyl, optionally substituted alkynyl, optionally substituted heteroalkyl, optionally substituted carbocyclyl, optionally substituted heterocyclyl, optionally substituted aryl, optionally substituted heteroaryl group, or an optionally substituted ring structure, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), C(O)N(Y₁)(Y₂) and S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O)(C₁-C₈)alkyl groups, —NHC(O)(C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(—NH)—(C₁-C₆)alkyl groups, —C(—NOH)—(C₁-C₆)alkyl groups, or —C(—N—O—(C₁-C₆)alkyl)-(C₁-C₆)alkyl groups.

Exemplary carbon atom substituents include, but are not limited to, deuterium, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

In some embodiments, unless specified or otherwise contrary from context, an optionally substituted group herein can be unsubstituted or substituted with 1-5 substituents, as valency permits, wherein, when substituted: (i) each substituent is independently selected from halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy, wherein each R^{e} and R^{f} is independently selected from the group consisting of H, (C₁-C₆)alkyl, (C₃-C₆)cycloalkyl, and (C₃-C₆)cycloalkyl(C₁-C₆)alkyl; or R^{e} and R^{f} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl; or (ii) two of the substituents are joined to form a 3-10 membered ring and the remaining substituent(s) are as defined in (i).

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure [symbol] indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of a bacterial infection, a fungal infection, a viral infection, mastocytosis, mast cell-mediated inflammation or an allergic disease. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention.

The term “therapeutically effective amount” or “effective amount” is an amount sufficient to effect beneficial or desired results such as clinical results. An effective amount can be administered in one or more administrations. An effective amount is typically sufficient to palliate, ameliorate, stabilize, reverse, slow or delay the progression of the disease state.

The term “mammal” refers to any mammalian species such as a human, mouse, rat, dog, cat, hamster, guinea pig, rabbit, livestock, and the like. Accordingly, in certain embodiments, the mammal is a human, mouse, rat, dog, cat, hamster, guinea pig, rabbit or livestock. In certain embodiments, the mammal is a patient (e.g., a human patient). In certain embodiments, the mammal is a pet, such a dog, cat, hamster, guinea pig or rabbit. In certain embodiments, the mammal is a livestock mammal (e.g., a cow, sheep, horse, pig, chicken, etc.).

The term “food” as used herein refers to a substance that can be consumed, e.g., to provide nutritional or therapeutic support to an organism. The term may include, but is not limited to, proteins, carbohydrates, fats, therapeutic agents, such as medicines, etc.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of Formula I or Formula A, e.g., any of the subformulae or specific compounds according to Formula I or Formula A as defined herein, or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkanoyloxy can be formyloxy, acetoxy, propanoyloxy, butanoyloxy, isobutanoyloxy, pentanoyloxy, or hexanoyloxy; and aryl can be phenyl, indenyl, or naphthyl.

### Formula I

Some embodiments of the present disclosure are directed to compounds of Formula I, as defined herein, or a pharmaceutically acceptable salt thereof.

In one specific embodiment, the compound of Formula I is a compound of Formula Ia:

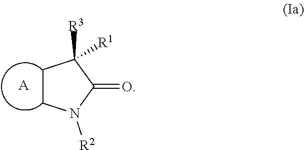

In one specific embodiment, the compound of Formula I is a compound of Formula Ib:

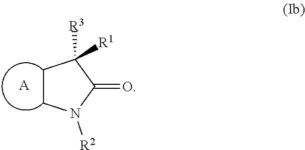

In one specific embodiment, R¹ is aryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.

In one specific embodiment, R¹ is 5-membered heteroaryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.

In one specific embodiment, R¹ is 6-membered heteroaryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.

In one specific embodiment, R¹ is (C₁-C₃)alkyl that is substituted with aryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.

In one specific embodiment, R¹ is (C₁-C₃)alkyl that is substituted with 5-membered heteroaryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.

In one specific embodiment, R¹ is (C₁-C₃)alkyl that is substituted with 6-membered heteroaryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.

In one specific embodiment, R¹ is phenyl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.

In one specific embodiment, R¹ is phenyl that is substituted with —S(═O)₂NH₂.

In one specific embodiment, R¹ is 4-(aminosulfonyl)phenyl.

In one specific embodiment, R² is benzyl that is optionally substituted with one or more groups independently selected from the group consisting of halo, hydroxy, cyano, nitro, (C₁-C₆)alkyl, (C₁-C₆)alkoxy, (C₁-C₆)alkanoyl, (C₁-C₆)alkoxycarbonyl, (C₁-C₆)alkanoyloxy, and NR^{c}R^{d}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.

In one specific embodiment, R² is benzyl that is optionally substituted with (C₁-C₆)alkoxy.

In one specific embodiment, R² is 4-methoxybenzyl.

In one specific embodiment, R³ is H.

In one specific embodiment, R³ is fluoro.

In one specific embodiment, R³ is (C₁-C₆)alkyl that is optionally substituted with one or more fluoro;

In one specific embodiment, R³ is (C₁-C₆)alkoxy that is optionally substituted with one or more fluoro;

In one specific embodiment, ring A is phenyl, and ring A is optionally substituted with 1, 2, 3, or 4 groups independently selected from the group consisting of halo, hydroxy, cyano, nitro, (C₁-C₆)alkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;

In one specific embodiment, ring A is 5-membered heteroaryl, and ring A is optionally substituted with 1, 2, 3, or 4 groups independently selected from the group consisting of halo, hydroxy, cyano, nitro, (C₁-C₆)alkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;

In one specific embodiment, ring A is 6-membered heteroaryl, and ring A is optionally substituted with 1, 2, 3, or 4 groups independently selected from the group consisting of halo, hydroxy, cyano, nitro, (C₁-C₆)alkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;

In one specific embodiment, the compound of Formula I is a compound of Formula IIa:

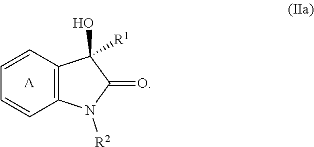

In one specific embodiment, the compound of Formula I is a compound of Formula IIb:

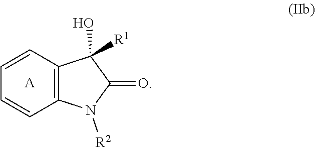

In one specific embodiment, the compound of Formula I is a compound of Formula IIIa:

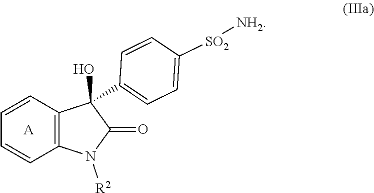

In one specific embodiment, the compound of Formula I is a compound of Formula IIIb:

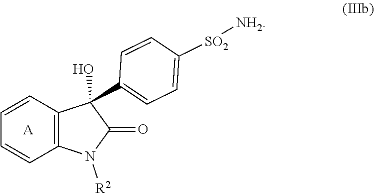

In one specific embodiment, the invention provides the compound:

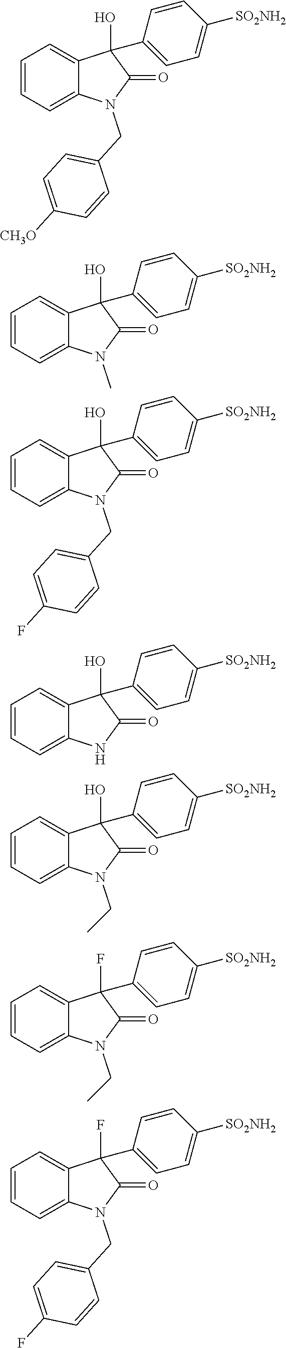

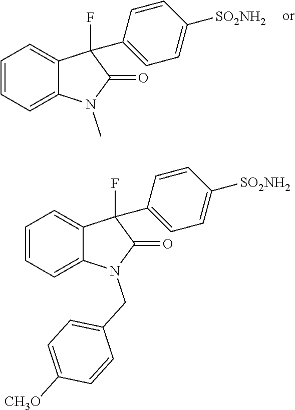

or a pharmaceutically acceptable salt thereof.

In one embodiment, aryl is phenyl or naphthyl.

In one embodiment, 5-membered heteroaryl is pyrrole, furan, thiophene, pyrazole, isoxazole, oxazole, isothiazole, thiazole, or triazole.

In one embodiment, 6-membered heteroaryl is pyridine, pyridazine, pyrimidine, or pyrazine.

### Formula A

Some embodiments of the present disclosure are directed to compounds of Formula A, or a pharmaceutically acceptable salt thereof:

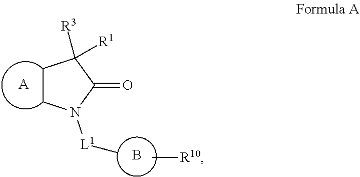

wherein the variables R¹, R³, L¹, R¹⁰, ring A, and ring B are defined herein.

In some embodiments, L¹ in Formula A is an optionally substituted C₁₋₃ alkylene, such as an optionally substituted methylene. When substituted, the C₁₋₃ alkylene can be substituted with 1-5 substituents as described herein. For example, in some embodiments, the C₁₋₃ alkylene can be substituted with 1, 2, 3, or 4 groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy, wherein each R^{e} and R^{f} is independently selected from the group consisting of H, (C₁-C₆)alkyl, (C₃-C₆)cycloalkyl, and (C₃-C₆)cycloalkyl(C₁-C₆)alkyl; or R^{e} and R^{f} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl.

In some embodiments, L¹ in Formula A is a C₁₋₃ alkylene, such as an methylene, which can be unsubstituted or substituted with an optionally substituted alkyl, for example, substituted with an optionally substituted C₁₋₃ alkyl.

For example, in some embodiments, the compound of Formula A can be characterized as having a structure according to Formula A-1:

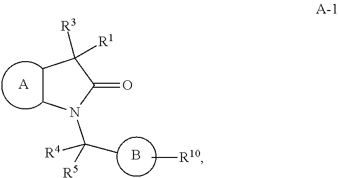

wherein:
R⁴ and R⁵ are each independently hydrogen or an optionally substituted C₁₋₃ alkyl. In some embodiments, R⁴ is hydrogen. In some embodiments, both R⁴ and R⁵ are hydrogen.

In some embodiments, L¹ in Formula A is absent.

Ring B in Formula A is typically a cyclic structure, although in some embodiments, ring B can also be absent, in which case, R¹⁰ is directly attached to L¹ or the nitrogen atom, when L¹ is also absent, in Formula A.

In some embodiments, ring B in Formula A can be an optionally substituted arylene, such as optionally substituted phenylene or optionally substituted naphthylene.

In some embodiments, ring B in Formula A can also be an optionally substituted heteroarylene, such as a 5 or 6-membered heteroarylene, or a 9 or 10 membered bicyclic heteroarylene.

In some embodiments, ring B in Formula A can also be an optionally substituted carbocyclylene.

In some embodiments, ring B in Formula A can also be an optionally substituted heterocyclylene.

For example, in some embodiments, the compound of Formula A-1 can have a ring B which is an optionally substituted phenylene or optionally substituted 5 or 6-membered heteroarylene. In some specific embodiments, ring B can be an unsubstituted phenylene.

In some embodiments, the compound of Formula A-1 can be characterized as having a structure according to formula A-1-A:

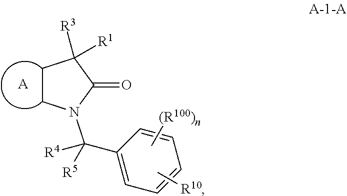

wherein:
n is 0, 1, 2, or 3; and
(i) R¹⁰⁰ at each occurrence is independently halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f} wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy, wherein each R^{e} and R^{f} is independently selected from the group consisting of H, (C₁-C₆)alkyl, (C₃-C₆)cycloalkyl, and (C₃-C₆)cycloalkyl(C₁-C₆)alkyl; or R^{e} and R^{f} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl; or
(ii) two adjacent R¹⁰⁰, together with the intervening atoms, are joined to form an optionally substituted 4-7 membered ring, which optionally contains a ring heteroatom and is aromatic or nonaromatic, and any remaining R¹⁰⁰ is defined in (i). For example, in some embodiments,

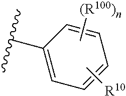

### in Formula A-1-A can be

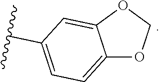 [SMILES: *c1ccc2c(c1)OCO2]

In some embodiments, the compound of Formula A-1 can be characterized as having a structure according to formula A-1-A:

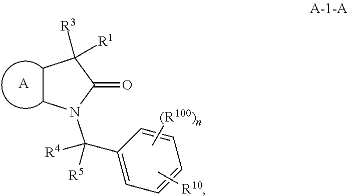

wherein:
n is 0, 1, 2, or 3; and
(i) R¹⁰⁰ at each occurrence is independently halogen (e.g., F), OH, C₁₋₄ alkyl, C₁₋₄ alkoxy, or C₁₋₄ heteroalkyl, wherein the C₁₋₄ alkyl, C₁₋₄ alkoxy, or C₁₋₄ heteroalkyl is optionally substituted with 1-3 fluorine; or
(ii) two adjacent R¹⁰⁰, together with the intervening atoms, are joined to form an optionally substituted 4-7 membered ring, which optionally contains a ring heteroatom and is aromatic or nonaromatic, and any remaining R¹⁰⁰ is defined in (i).
   R can be at an ortho, meta, or para position to the C(R⁴)(R⁵) unit in Formula A-1-A. For example, in some preferred embodiments,

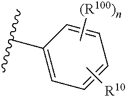

### in Formula A-1-A can be

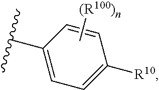

wherein R¹⁰, R¹⁰⁰ and n are as defined herein.

In some embodiments, n in Formula A-1-A is 0. In some embodiments, n in Formula A-1-A is 1 or 2.

In some embodiments, ring B in Formula A, such as Formula A-1, can be an optionally substituted 5 or 6 membered heteroarylene having 1-4 ring heteroatoms, wherein each ring heteroatom is independently nitrogen, oxygen, or sulfur. In some embodiments, the 5 or 6 membered heteroarylene can be a pyridylene. In some embodiments, a ring nitrogen atom of the 5 or 6 membered heteroarylene is optionally oxidized. Typically, when substituted, the 5 or 6 membered heteroarylene is preferably substituted with 1-3 substituents as valency permits. For example, in some embodiments, the 5 or 6 membered heteroarylene can be substituted with 1-3 substituents, as valency permits, each independently selected from halogen (e.g., F), OH, C₁₋₄ alkyl, C₁₋₄ alkoxy, C₁₋₄ heteroalkyl, or a 3-6 membered ring, wherein the C₁₋₄ alkyl, C₁₋₄ alkoxy, or C₁₋₄ heteroalkyl is optionally substituted with 1-3 fluorine.

In some embodiments, ring B in Formula A, such as Formula A-1, can be an optionally substituted bicyclic heteroarylene (e.g., a 9 or 10-membered bicyclic heteroarylene, such as a 5,6-bicyclic or 6,6-bicyclic heteroarylene), which has 1-4 ring heteroatoms, wherein each ring heteroatom is independently nitrogen, oxygen, or sulfur. In some embodiments, the bicyclic heteroarylene can be a 6,6-bicyclic heteroarylene, such as a quinoline or isoquinoline ring. In some embodiments, a ring nitrogen atom of the bicyclic heteroarylene is optionally oxidized. Typically, when substituted, the bicyclic heteroarylene is preferably substituted with 1-3 substituents as valency permits. For example, in some embodiments, the bicyclic heteroarylene can be substituted with 1-3 substituents, as valency permits, each independently selected from halogen (e.g., F), OH, C₁₋₄ alkyl, C₁₋₄ alkoxy, C₁₋₄ heteroalkyl, or a 3-6 membered ring, wherein the C₁₋₄ alkyl, C₁₋₄ alkoxy, or C₁₋₄ heteroalkyl is optionally substituted with 1-3 fluorine.

In some embodiments, ring B in Formula A, such as in Formula A-1, can be an optionally substituted 4-7 membered monocyclic heterocyclic ring having 1 or 2 ring heteroatoms, wherein each ring heteroatom is independently nitrogen, oxygen, or sulfur. For example, in some embodiments, the 4-7 membered monocyclic heterocyclic ring can be a tetrahydropyran ring. Typically, when substituted, the 4-7 membered monocyclic heterocyclic ring is preferably substituted with 1-3 substituents. For example, in some embodiments, the 4-7 membered monocyclic heterocyclic ring can be substituted with 1-3 substituents each independently selected from oxo, halogen (e.g., F), OH, C₁₋₄ alkyl, C₁₋₄ alkoxy, C₁₋₄ heteroalkyl, or a nitrogen protecting group, wherein the C₁₋₄ alkyl, C₁₋₄alkoxy, or C₁₋₄ heteroalkyl is optionally substituted with 1-3 fluorine.

Various groups are suitable for R¹⁰ in Formula A. In some embodiments according to Formula A, such as Formula A-1, R¹⁰ can be hydrogen. In some embodiments according to Formula A, such as Formula A-1, R¹⁰ can be a halo group. In some embodiments according to Formula A, R¹⁰ can be G¹ or O-G¹ as defined herein. Other suitable definitions of R¹⁰ are described herein.

In some embodiments, R¹⁰ in Formula A, such as in Formula A-1 or A-1-A, is an optionally substituted 4-7 membered monocyclic heterocyclic ring having 1 or 2 ring heteroatoms, wherein each ring heteroatom is independently nitrogen, oxygen, or sulfur. Typically, when substituted, the 4-7 membered monocyclic heterocyclic ring is preferably substituted with 1-3 substituents. For example, in some embodiments, the 4-7 membered monocyclic heterocyclic ring can be substituted with 1-3 substituents each independently selected from oxo, halogen (e.g., F), OH, C₁₋₄ alkyl, C₁₋₄ alkoxy, C₁₋₄ heteroalkyl, a 3-6 membered ring, or a nitrogen protecting group (as applicable), wherein the C₁₋₄ alkyl, C₁₋₄ alkoxy, or C₁₋₄ heteroalkyl is optionally substituted with 1-3 fluorine.

In some embodiments, R¹⁰ in Formula A, such as in Formula A-1 or A-1-A, is an optionally substituted 5 or 6 membered monocyclic heterocyclic ring having 1 or 2 ring heteroatoms, wherein each ring heteroatom is independently nitrogen, oxygen, or sulfur. For example, in some embodiments, the 5 or 6 membered monocyclic heterocyclic ring can be pyrrolidine, piperidine, piperazine, morpholine, etc., which can be attached to the remainder of the molecule through a ring carbon atom or a ring nitrogen atom. Typically, when substituted, the 5 or 6 membered monocyclic heterocyclic ring is preferably substituted with 1-3 substituents. For example, in some embodiments, the 5 or 6 membered monocyclic heterocyclic ring can be substituted with 1-3 substituents each independently selected from oxo, halogen (e.g., F), OH, C₁₋₄ alkyl, C₁₋₄ alkoxy, C₁₋₄ heteroalkyl, a 3-6 membered ring, or a nitrogen protecting group (as applicable), wherein the C₁₋₄ alkyl, C₁₋₄ alkoxy, or C₁₋₄ heteroalkyl is optionally substituted with 1-3 fluorine. In some specific embodiments, R¹⁰ in Formula A, such as in Formula A-1 or A-1-A, can be selected from:

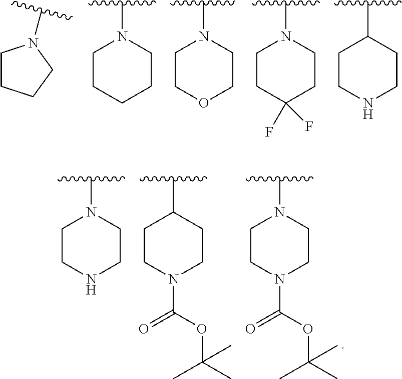

In some embodiments, R¹⁰ in Formula A, such as in Formula A-1 or A-1-A, is an optionally substituted 5 or 6 membered heteroaryl ring having 1-4 ring heteroatoms, wherein each ring heteroatom is independently nitrogen, oxygen, or sulfur. For example, in some embodiments, the 5 or 6 membered heteroaryl is a pyridyl or imidazole ring. In some embodiments, a ring nitrogen atom of the 5 or 6 membered heteroaryl can be optionally oxidized. Typically, when substituted, the 5 or 6 membered heteroaryl ring is preferably substituted with 1-3 substituents as valency permits. For example, in some embodiments, the 5 or 6 membered heteroaryl ring is substituted with 1-3 substituents each independently selected from halogen (e.g., F), OH, C₁₋₄ alkyl, C₁₋₄ alkoxy, C₁₋₄ heteroalkyl, or a 3-6 membered ring, wherein the C₁₋₄ alkyl, C₁₋₄ alkoxy, or C₁₋₄ heteroalkyl is optionally substituted with 1-3 fluorine. In some specific embodiments, R¹⁰ in Formula A, such as in Formula A-1 or A-1-A, can be

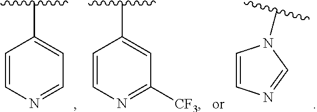

In some embodiments, the compound of Formula A can be characterized as having a structure according to Formula A-2:

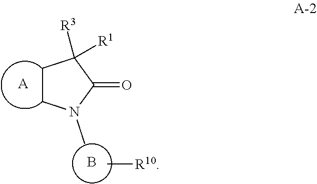

Ring B in Formula A-2 can be any of those ring B as described herein, such as those described hereinabove in connection with Formula A-1.

In some embodiments, ring B in Formula A-2 is an optionally substituted phenylene, optionally substituted heteroarylene (e.g., 5-10 membered heteroarylene, such as isoquinolinylene), or an optionally substituted naphthylene.

In some embodiments, ring B in Formula A-2 can be an optionally substituted 5 or 6 membered heteroarylene having 1-4 ring heteroatoms, wherein each ring heteroatom is independently nitrogen, oxygen, or sulfur. In some embodiments, the 5 or 6 membered heteroarylene can be a pyridylene. In some embodiments, a ring nitrogen atom of the 5 or 6 membered heteroarylene is optionally oxidized. Typically, when substituted, the 5 or 6 membered heteroarylene is preferably substituted with 1-3 substituents as valency permits. For example, in some embodiments, the 5 or 6 membered heteroarylene can be substituted with 1-3 substituents, as valency permits, each independently selected from halogen (e.g., F), OH, C₁₋₄ alkyl, C₁₋₄ alkoxy, C₁₋₄ heteroalkyl, or a 3-6 membered ring, wherein the C₁₋₄ alkyl, C₁₋₄ alkoxy, or C₁₋₄ heteroalkyl is optionally substituted with 1-3 fluorine.

In some embodiments, ring B in Formula A-2 can be an optionally substituted bicyclic heteroarylene (e.g., a 9 or 10-membered bicyclic heteroarylene, such as a 5,6-bicyclic or 6,6-bicyclic heteroarylene), which has 1-4 ring heteroatoms, wherein each ring heteroatom is independently nitrogen, oxygen, or sulfur. In some embodiments, the bicyclic heteroarylene can be a 6,6-bicyclic heteroarylene, such as a quinoline or isoquinoline ring. In some embodiments, a ring nitrogen atom of the bicyclic heteroarylene is optionally oxidized. Typically, when substituted, the bicyclic heteroarylene is preferably substituted with 1-3 substituents as valency permits. For example, in some embodiments, the bicyclic heteroarylene can be substituted with 1-3 substituents, as valency permits, each independently selected from halogen (e.g., F), OH, C₁₋₄ alkyl, C₁₋₄ alkoxy, C₁₋₄ heteroalkyl, or a 3-6 membered ring, wherein the C₁₋₄ alkyl, C₁₋₄ alkoxy, or C₁₋₄ heteroalkyl is optionally substituted with 1-3 fluorine.

For example, in some embodiments, the compound of Formula A-2 can be characterized as having a structure according to Formula A-2-A, A-2-B, or A-2-C:

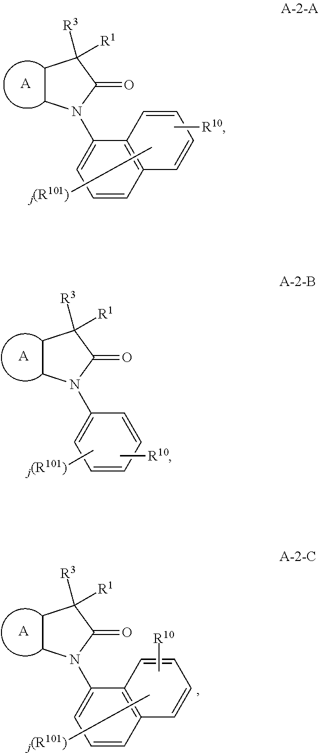

wherein:
j is 0, 1, 2, 3, or 4; and
(i) R¹⁰¹ at each occurrence is independently halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy, wherein each R^{e} and R^{f} is independently selected from the group consisting of H, (C₁-C₆)alkyl, (C₃-C₆)cycloalkyl, and (C₃-C₆)cycloalkyl(C₁-C₆)alkyl; or R^{e} and R^{f} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl; or
(ii) two instances of R¹⁰¹ are joined together to form a 5-7 membered ring, which is optionally substituted with halogen, CN, C₁₋₄ alkyl optionally substituted with 1-3 F, C₁₋₄ alkoxy optionally substituted with 1-3 F, or C₁₋₄ heteroalkyl optionally substituted with 1-3 F, and any remaining R¹⁰¹ is defined in (i). For example, in some embodiments,

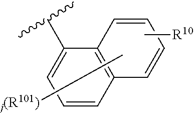

### in Formula A-2-A can be

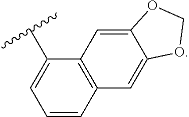 [SMILES: *c1cccc2cc3c(cc12)OCO3]

To be clear, in Formula A-2-A and A-2-C, it should be understood that the R¹⁰¹ group(s) and R¹⁰ group can be attached to any available position at either of the two rings.

In some embodiments, the compound of Formula A-2 can be characterized as having a structure according to Formula A-2-A, A-2-B, or A-2-C:

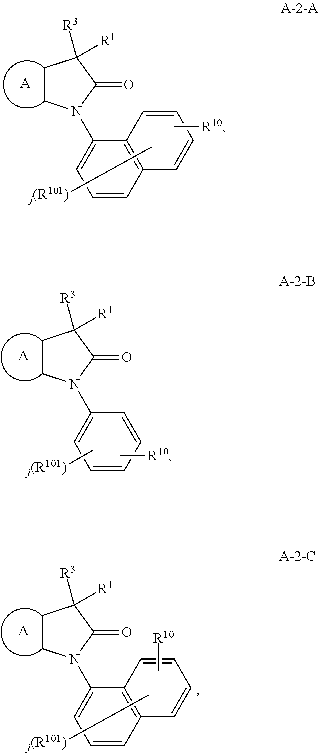

wherein:
j is 0, 1, 2, 3, or 4; and
(i) R¹⁰¹ at each occurrence is independently halogen, CN, OH, G², or OG², wherein G² at each occurrence is independently C₁₋₄ alkyl, C₁₋₄ heteroalkyl, 3-6 membered ring, (C₁₋₄ alkylene)-(3-6 membered ring), or (C₁₋₄ heteroalkylene)-(3-6 membered ring), wherein the C₁₋₄ alkyl, C₁₋₄ heteroalkyl, C₁₋₄ alkylene, or C₁₋₄ heteroalkylene, is optionally substituted with 1-3 fluorine; and the 3-6 membered ring is optionally substituted with halogen, CN, C₁₋₄ alkyl optionally substituted with 1-3 F, C₁₋₄ alkoxy optionally substituted with 1-3 F, or C₁₋₄ heteroalkyl optionally substituted with 1-3 F, or
(ii) two instances of R¹⁰¹ are joined together to form a 5-7 membered ring, which is optionally substituted with halogen, CN, C₁₋₄ alkyl optionally substituted with 1-3 F, C₁₋₄ alkoxy optionally substituted with 1-3 F, or C₁₋₄ heteroalkyl optionally substituted with 1-3 F, and any remaining R¹⁰¹ is defined in (i). To be clear, in Formula A-2-A and A-2-C, it should be understood that the R¹⁰¹ group(s) and R¹⁰ group can be attached to any available position at either of the two rings.

In Formula A-2-A, R¹⁰ can be at any position of the naphthyl ring. For example, in some preferred embodiments,

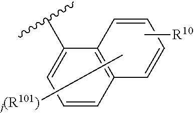

### in Formula A-2-A can be

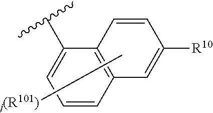

wherein R¹⁰, R¹⁰¹ and j are as defined herein.

In Formula A-2-B, R¹⁰ can be at an ortho, meta, or para position to the amide (N(CO)) unit. For example, in some preferred embodiments,

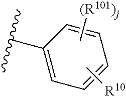

### in Formula A-2-B can be

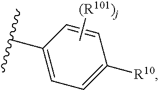

wherein R¹⁰, R¹⁰¹ and j are as defined herein.

In some embodiments, R¹⁰ can be hydrogen and

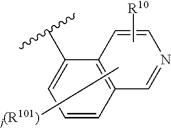

in Formula A-2-C can be represented by

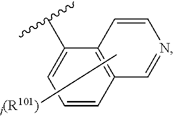

wherein R¹⁰¹ and j are as defined herein.

Suitable R¹⁰ groups for Formula A-2 such as A-2-A, A-2-B, or A-2-C include any of those described herein, such as those described in connection with Formula A-1. In some embodiments, R¹⁰ in Formula A-2 such as A-2-A, A-2-B, or A-2-C can be hydrogen, halogen, OH, or C₁₋₄ alkoxy optionally substituted with 1-3 F. For example, in some embodiments, R¹⁰ in Formula A-2 such as A-2-A, A-2-B, or A-2-C can be hydrogen. In some embodiments, R¹⁰ in Formula A-2 such as A-2-A, A-2-B, or A-2-C can be F or Cl. In some embodiments, R¹⁰ in Formula A-2 such as A-2-A, A-2-B, or A-2-C can be a an optionally substituted 5 or 6 membered monocyclic heterocyclic ring having 1 or 2 ring heteroatoms, wherein each ring heteroatom is independently nitrogen, oxygen, or sulfur, such as morpholine,

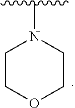 [SMILES: *N1CCOCC1]

When substituted, the 5 or 6 membered monocyclic heterocyclic ring is preferably substituted with 1-3 substituents, such as 1-3 substituents each independently selected from oxo, halogen (e.g., F), OH, C₁₋₄ alkyl, C₁₋₄ alkoxy, C₁₋₄ heteroalkyl, a 3-6 membered ring, or a nitrogen protecting group, wherein the C₁₋₄ alkyl, C₁₋₄ alkoxy, or C₁₋₄ heteroalkyl is optionally substituted with 1-3 fluorine.

Suitable R¹, R³, and ring A for Formula A (e.g., A-1, A-1-A, A-2, A-2-A, A-2-B, or A-2-C) include any of those described herein in connection with formula I and its subformulae. For example, in some embodiments, R¹ in Formula A can be

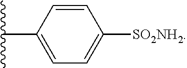 [SMILES: *c1ccc(S(N)(=O)=O)cc1]

In some embodiments, R³ in Formula A can be OH. In some embodiments, ring A in Formula A can be a phenyl ring. For example, in some embodiments, the compound of Formula A can be characterized as having a structure according to Formula A-3:

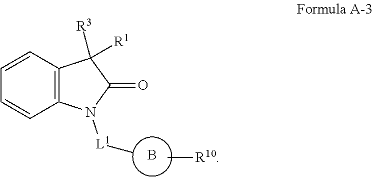

In some embodiments, the compounds of Formula A (including any of the subformulae herein) can be characterized as having certain stereochemistry. For example, in some embodiments, the compound of Formula A can have a stereochemistry according to Formula A-E1 or A-E2:

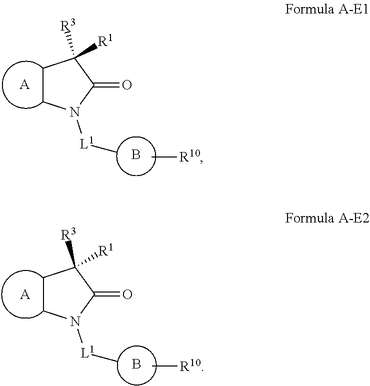

In some embodiments, the present disclosure also provides novel specific compounds as described in the Examples section herein, including any new synthetic intermediate or target compound, as well as a stereoisomer thereof, a deuterated analog thereof, or a pharmaceutically acceptable salt thereof.

In some embodiments, the present disclosure provides a compound selected from the following table, a stereoisomer thereof, a deuterated analog thereof, or a pharmaceutically acceptable salt thereof.

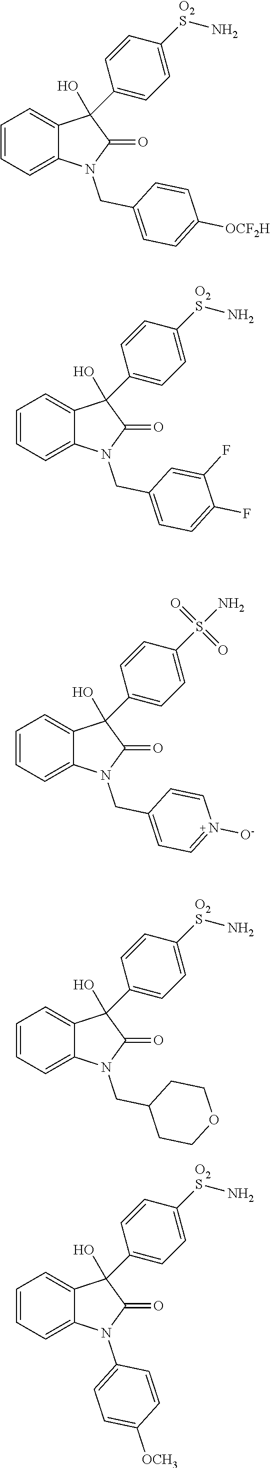

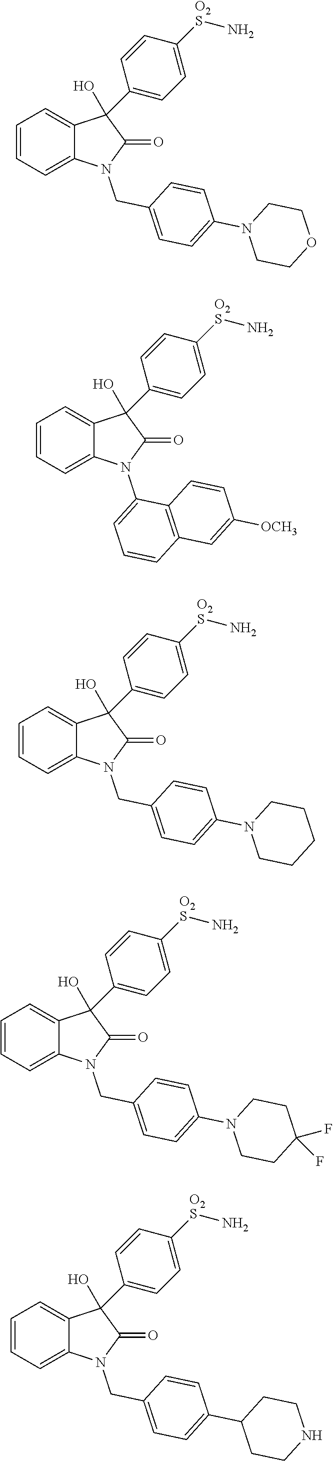 [SMILES: NS(=O)(=O)c1ccc(C2(O)C(=O)N(Cc3ccc(N4CCOCC4)cc3)c3ccccc32)cc1 ; COc1ccc2c(N3C(=O)C(O)(c4ccc(S(N)(=O)=O)cc4)c4ccccc43)cccc2c1 ; NS(=O)(=O)c1ccc(C2(O)C(=O)N(Cc3ccc(N4CCCCC4)cc3)c3ccccc32)cc1 ; NS(=O)(=O)c1ccc(C2(O)C(=O)N(Cc3ccc(N4CCC(F)(F)CC4)cc3)c3ccccc32)cc1 ; NS(=O)(=O)c1ccc(C2(O)C(=O)N(Cc3ccc(C4CCNCC4)cc3)c3ccccc32)cc1]

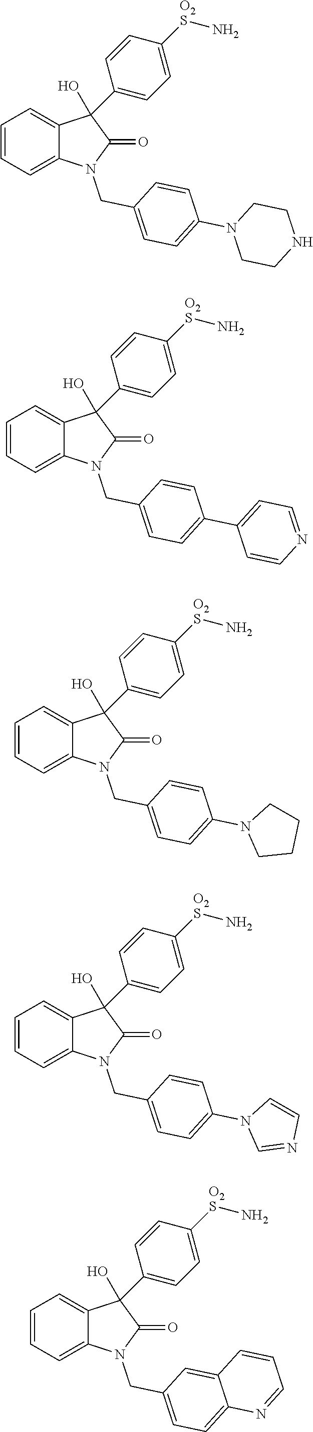 [SMILES: NS(=O)(=O)c1ccc(C2(O)C(=O)N(Cc3ccc(N4CCNCC4)cc3)c3ccccc32)cc1 ; NS(=O)(=O)c1ccc(C2(O)C(=O)N(Cc3ccc(-c4ccncc4)cc3)c3ccccc32)cc1 ; NS(=O)(=O)c1ccc(C2(O)C(=O)N(Cc3ccc(N4CCCC4)cc3)c3ccccc32)cc1 ; NS(=O)(=O)c1ccc(C2(O)C(=O)N(Cc3ccc(-n4ccnc4)cc3)c3ccccc32)cc1 ; NS(=O)(=O)c1ccc(C2(O)C(=O)N(Cc3ccc4ncccc4c3)c3ccccc32)cc1]

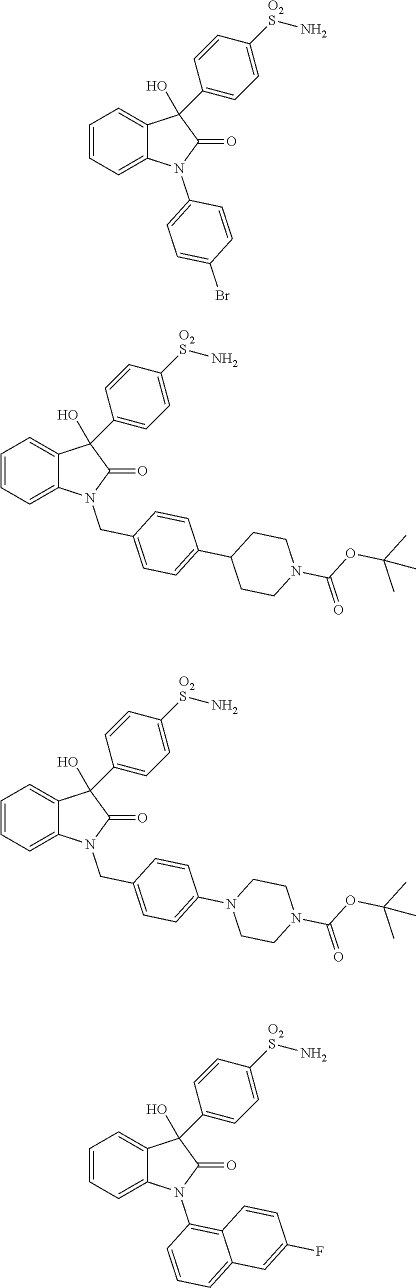 [SMILES: NS(=O)(=O)c1ccc(C2(O)C(=O)N(c3ccc(Br)cc3)c3ccccc32)cc1 ; CC(C)(C)OC(=O)N1CCC(c2ccc(CN3C(=O)C(O)(c4ccc(S(N)(=O)=O)cc4)c4ccccc43)cc2)CC1 ; CC(C)(C)OC(=O)N1CCN(c2ccc(CN3C(=O)C(O)(c4ccc(S(N)(=O)=O)cc4)c4ccccc43)cc2)CC1 ; NS(=O)(=O)c1ccc(C2(O)C(=O)N(c3cccc4cc(F)ccc34)c3ccccc32)cc1]

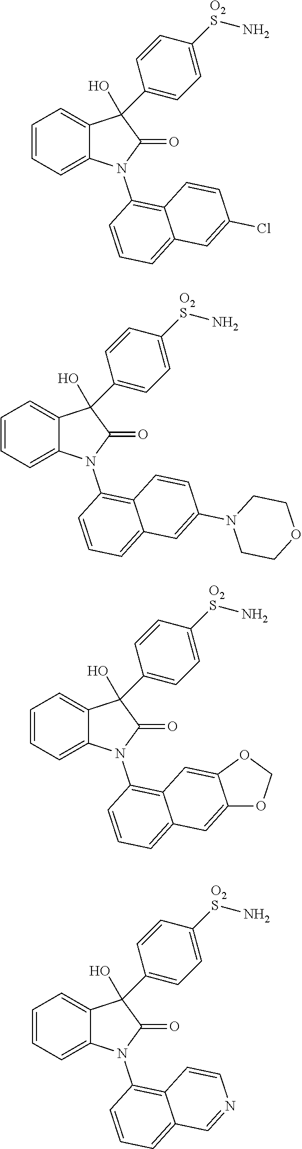 [SMILES: NS(=O)(=O)c1ccc(C2(O)C(=O)N(c3cccc4cc(Cl)ccc34)c3ccccc32)cc1 ; NS(=O)(=O)c1ccc(C2(O)C(=O)N(c3cccc4cc(N5CCOCC5)ccc34)c3ccccc32)cc1 ; NS(=O)(=O)c1ccc(C2(O)C(=O)N(c3cccc4cc5c(cc34)OCO5)c3ccccc32)cc1 ; NS(=O)(=O)c1ccc(C2(O)C(=O)N(c3cccc4cnccc34)c3ccccc32)cc1]

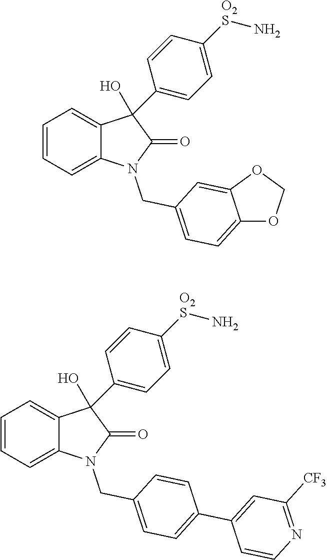 [SMILES: NS(=O)(=O)c1ccc(C2(O)C(=O)N(Cc3ccc4c(c3)OCO4)c3ccccc32)cc1 ; NS(=O)(=O)c1ccc(C2(O)C(=O)N(Cc3ccc(-c4ccnc(C(F)(F)F)c4)cc3)c3ccccc32)cc1]

In cases where compounds are sufficiently basic or acidic, a salt of a compound of Formula I or Formula A can be useful as an intermediate for isolating or purifying a compound of Formula I or Formula A. Additionally, administration of a compound of Formula I or Formula A as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of the present disclosure can be readily synthesized by those skilled in the art in view of the present disclosure. Exemplified synthesis are also shown in the Examples section.

### Certain Methods of Use

As described herein, compounds of Formula I or Formula A may be used as carbonic anhydrase inhibitors. Carbonic anhydrases (Car) are a family of metabolic enzymes that regulate pH and CO₂ homeostasis (Supuran, C. T. 2008. Nat Rev Drug Discov 7:168-181, which is hereby incorporated by reference herein). The Car enzymes found in mammals are divided into four broad subgroups, which, in turn consist of several isoforms: cytosolic Car (Car1, Car2, Car3, Car7, Car13), mitochondrial Car (Car5A, Car5B), secreted Car (Car6), and membrane-associated Car (Car4, Car9, Car12, Car14). Additionally, there are three additional “acatalytic” Car isoforms (Car8, Car10, Car11) whose functions remain unclear.

Accordingly, certain embodiments of the invention provide a method of inhibiting a carbonic anhydrase enzyme (e.g., the enzyme's activity or function) in vitro or in vivo, the method comprising contacting the carbonic anhydrase enzyme with an effective amount of a compound of Formula I or Formula A, or a salt thereof (e.g., a pharmaceutically acceptable salt thereof). In certain embodiments, such a method comprises contacting a cell comprising the carbonic anhydrase enzyme. In certain embodiments, the cell is in a mammal. In certain embodiments, the cell is contacted by administering the compound of Formula I or Formula A, a salt thereof (e.g., a pharmaceutically acceptable salt thereof) to the mammal.

In certain embodiments, the activity or function of the carbonic anhydrase is inhibited by at least about 5%, at least about 10%, at least about 20%, at least about 30%, at least about 40%, at least about 50%, at least about 60%, at least about 70%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% (e.g., as compared to a control, such as a cell or a mammal not contacted with the compound of Formula I or Formula A).

Certain embodiments also provide a compound of Formula I or Formula A, or a salt thereof, for use in inhibiting a carbonic anhydrase enzyme in vitro or in vivo.

Certain embodiments provide the use of a compound of Formula I or Formula A, or a salt thereof, for the manufacture of a medicament for inhibiting a carbonic anhydrase enzyme in vitro or in vivo.

In certain embodiments, the carbonic anhydrase enzyme is carbonic anhydrase (Car) 1 (see, UniProKB No P00915).

Certain embodiments also provide a method of inhibiting mast cell development and/or mast cell function (e.g., mast cell activation) in a mammal, comprising administering a compound of Formula I or Formula A, or a pharmaceutically acceptable salt thereof, to the mammal.

Certain embodiments provide a compound of Formula I or Formula A, or a pharmaceutically acceptable salt thereof, for use in the inhibition of mast cell development and/or mast cell function (e.g., mast cell activation).

Certain embodiments also provide the use of a compound of Formula I or Formula A, or a pharmaceutically acceptable salt thereof, to prepare a medicament for inhibiting mast cell development and/or mast cell function (e.g., mast cell activation).

Certain embodiments also provide a method of treating a disease or condition mediated by a carbonic anhydrase enzyme in a mammal (e.g., a human), comprising administering a compound of Formula I or Formula A, or a pharmaceutically acceptable salt thereof, to the mammal.

Certain embodiments provide a compound of Formula I or Formula A, or a pharmaceutically acceptable salt thereof, for the prophylactic or therapeutic treatment of a disease or condition mediated by a carbonic anhydrase enzyme.

Certain embodiments also provide the use of a compound of Formula I or Formula A, or a pharmaceutically acceptable salt thereof, to prepare a medicament for treating a disease or condition mediated by a carbonic anhydrase enzyme.

Examples of diseases or conditions mediated by a carbonic anhydrase enzyme (e.g., mediated by carbonic anhydrase enzyme activity/function) are known in the art, and include, e.g., mast cell-mediated diseases. For example, diseases associated with carbonic anhydrase enzymes and/or mast cells include, but are not limited to, e.g., allergic diseases, bacterial infections, fungal infections, viral infections, mastocytosis and mast cell-mediated inflammation.

In certain embodiments, the disease or condition mediated by a carbonic anhydrase enzyme is an allergic disease.

The term “allergic disease” refers to a condition caused by hypersensitivity of the immune system in response to an environmental exposure. Examples of the allergic diseases that can be treated include, but are not limited to, asthma (e.g. fungal asthma), atopic dermatitis, contact dermatitis, chronic itch (pruritus), urticaria, hay fever, allergic conjunctivitis, allergic rhinitis, anaphylaxis, eosinophilic esophagitis, food allergies, and allergen-induced mastocytosis.

In certain other embodiments, the disease or condition mediated by a carbonic anhydrase enzyme is a bacterial, fungal or viral infection.

Non-limiting examples of bacterial infections include, e.g., Klebsiella pneumoniae and Pseudomonas aeruginosa. In certain embodiments, the bacterial infection is a gram-negative bacterial infection.

Non-limiting examples of fungal infections include infections caused by Aspergillus fumigates, or other Aspergillus species, as well as Candida species, Cryptococcus species, Histoplasma capsulatum, Pneumocystis jirovecii, and Stachybotrys chartarum.

Non-limiting examples of viral infections include HIV, SARS-CoV-2, and Dengue.

In another aspect, the disease or condition mediated by a carbonic anhydrase enzyme is mastocytosis.

As used herein, the term “mastocytosis” refers to a disease characterized by the presence of too many mast cells in various organs and tissues, including but not limited to, infection- or allergen-induced mastocytosis, cutaneous mastocytosis, indolent systemic mastocytosis, systemic mastocytosis with associated clonal hematologic non-mast cell lineage diseases (such as myelodysplastic syndrome, myeloproliferative syndrome, acute myeloid leukemia, non-Hodgkin's lymphoma), aggressive systemic mastocytosis, mast cell leukemia, mast cell activation syndrome, and localized mast cell proliferations (such as mast cell sarcoma and extracutaneous mastocytoma). In certain embodiments, the mastocytosis is mast cell activation syndrome.

In another aspect, the disease or condition mediated by a carbonic anhydrase enzyme is mast cell-mediated inflammation.

As used herein, the term “mast cell-mediated inflammation” refers to any inflammatory response and/or pathology that is promoted or supported by mast cell development and/or activation. This includes mast cell responses promoted by exposure to allergens, infectious agents, or unknown stimuli. In certain embodiments, the mast cell-mediated inflammation is caused by mastocytosis, an infection (e.g., a parasite infection, such as a helminth parasite (e.g., a Trichinella spiralis infection)), or an allergy (e.g., a food allergy or food allergy-like disease). In certain embodiments, the mast cell-mediated inflammation is mast cell-mediated intestinal inflammation. In certain embodiments, the mast cell-mediated inflammation is mast cell-mediated airway inflammation (e.g., eosinophilic airway inflammation).

In certain embodiments, administration of a compound of Formula I or Formula A, or a pharmaceutically acceptable salt thereof, reduces a mast cell response, such as mast cell-mediated inflammation (e.g., mast cell-mediated intestinal inflammation; or mast cell-mediated airway inflammation). In certain embodiments, the mast cell response (e.g., mast cell-mediated inflammation) is induced by an allergy, such as a food allergy.

In certain embodiments, a mast cell response (e.g., mast cell activation and/or mast-cell mediated inflammation) causes airway constriction. In certain embodiments, administration of a compound of Formula I or Formula A, or a pharmaceutically acceptable salt thereof, is capable of treating such airway constriction.

In certain embodiments, a method described herein may further comprise administering one or more additional therapeutic agent(s). For example, such as an agent may be useful for treating a disease or condition mediated by a carbonic anhydrase enzyme (e.g., for treating allergic diseases, bacterial infections, fungal infections, viral infections, mastocytosis and/or mast cell-mediated inflammation).

In certain embodiments, the one or more additional therapeutic agent(s) is/are administered simultaneously or sequentially with a compound of Formula I or Formula A, or a pharmaceutically acceptable salt thereof. In certain embodiments, the one or more additional therapeutic agent(s) is/are administered simultaneously with a compound of Formula I or Formula A, or a pharmaceutically acceptable salt thereof. In certain embodiments, a pharmaceutical composition/formulation comprising a compound of Formula I or Formula A, or a pharmaceutically acceptable salt thereof, and the one or more additional therapeutic agent(s) is administered. In certain embodiments, a compound of Formula I or Formula A, or a pharmaceutically acceptable salt thereof, and the one or more additional therapeutic agent(s) are administered sequentially. In certain embodiments, the compound of Formula I or Formula A, or a pharmaceutically acceptable salt thereof, is administered first and the one or more additional therapeutic agent(s) is administered second. In certain embodiments the one or more additional therapeutic agent(s) is administered first and the compound of Formula I or Formula A, or a pharmaceutically acceptable salt thereof, is administered second.

In certain embodiments, the one or more additional therapeutic agent(s) is an anti-histamine, a steroid, immunotherapy (e.g., an allergy shot, oral tolerance therapy, etc.), a decongestant, a bronchodilator, a mast cell stabilizer, a prostaglandin antagonist, a blocking/neutralizing antibody (e.g. an anti-IgE therapy, an anti-IL-4R alpha therapy, or an anti-SIGLEC8 therapy), and/or a leukotriene modifier. In certain embodiments, a combination of such agents is administered.

In certain embodiments, the one or more additional therapeutic agent(s) is an anti-histamine. A histamine antagonist, commonly called an antihistamine, is a pharmaceutical drug class that includes two types of drugs: histamine H₁-receptor antagonists and histamine H₂-receptor antagonists. Antagonists of the histamine H₁-receptor are used to treat allergic reactions in the nose (e.g., itching, runny nose, and sneezing) and which are used off-label for insomnia. They are sometimes also used to treat motion sickness or vertigo caused by problems with the inner ear. Antagonists of the histamine H₂-receptor are used to treat gastric acid conditions (e.g., peptic ulcers and acid reflux). They work by binding to histamine H₁ receptors in mast cells, smooth muscle, and endothelium in the body and tuberomammillary nucleus the brain or histamine H₂ receptors in the upper gastrointestinal tract, primarily in the stomach.

Non-limiting examples of antihistamines that may be used in the present invention include: acrivastine, azelastine, bilastine, brompheniramine, buclizine, bromodiphenhydramine, carbinoxamine, cetirizine (Zyrtec; metabolite of hydroxyzine, its prodrug), chlorpromazine, cimetidine, cyclizine, chlorphenamine, chlorodiphenhydramine, clemastine, cyproheptadine, desloratadine, dexbrompheniramine, dexchlorpheniramine, dimetindene, diphenhydramine (Benadryl), ebastine, embramine, famotidine, fexofenadine (Allegra), hydroxyzine (Vistaril), lafutidine, levocetirizine, loratadine (Claritin), nizatidine, olopatadine, phenindamine, pheniramine, phenyltoloxamine, promethazine, pyrilamine, ranitidine, roxatidine, rupatadine, tiotidine, tripelennamine, and triprolidine.

In certain embodiments, the one or more additional therapeutic agent(s) is a blocking and/or neutralizing antibody (e.g., an antibody useful for treating inflammation). For example, such an antibody may target IgE, IL-4R alpha or SIGLEC8.

Thus, in certain embodiments, the one or more additional therapeutic agent(s) is an anti-IgE therapy. Anti-IgE therapies that may be used in the present invention include anti-IgE therapeutic antibodies, such as a monoclonal antibody. A non-limiting example of a suitable monoclonal antibody is Omalizumab (Xolair).

In certain embodiments, the one or more additional therapeutic agent(s) is an anti-IL-4R alpha therapy. Anti-IL-4R therapies that may be used in the present invention include anti-IL-4R alpha therapeutic antibodies, such as a monoclonal antibody. A non-limiting example of a suitable antibody is dupilumab.

In certain embodiments, the one or more additional therapeutic agent(s) is an anti-SIGLEC8 alpha therapy. Anti-SIGLEC8 therapies that may be used in the present invention include anti-SIGLEC8 alpha therapeutic antibodies, such as a monoclonal antibody. A non-limiting example of a suitable antibody is lirentelimab.

In certain embodiments, the one or more additional therapeutic agent(s) is a prostaglandin antagonist. A prostaglandin antagonist is a hormone antagonist acting upon one or more prostaglandins, a subclass of eicosanoid compounds, which function as signaling molecules in numerous types of animal tissues. Non-limiting examples of prostaglandin antagonists that may be used in the present invention include NSAIDs and seratrodast.

In certain embodiments, the one or more additional therapeutic agent(s) a steroid. A steroid is an organic compound, typically containing four rings arranged in a specific configuration. Steroids have two principal biological functions: certain steroids (such as cholesterol) are important components of cell membranes which alter membrane fluidity, and many steroids are signaling molecules which activate steroid hormone receptors. Generally, the steroid core structure is composed of seventeen carbon atoms, bonded in four “fused” rings: three six-member cyclohexane rings (rings A, B and C in the first illustration) and one five-member cyclopentane ring (the D ring). Steroids vary by the functional groups attached to this four-ring core and by the oxidation state of the rings. Sterols are forms of steroids with a hydroxyl group at position three and a skeleton derived from cholestane. Steroids can also vary more markedly by changes to the ring structure (for example, ring scissions which produce secosteroids, such as vitamin D₃).

Non-limiting examples of steroids that may be used in the present invention include: beclomethasone, ciclesonide, fluticasone proprionate, fluticasone furoate, mometasone, budesonide, triamcinolone, dexamethasone, deltasone, and prednisone.

In certain embodiments, the one or more additional therapeutic agent(s) is an immunotherapy. Immunotherapy is the treatment of a disease or condition by inducing, enhancing, or suppressing an immune response. Immunotherapies that are designed to elicit or amplify an immune response are classified as activation immunotherapies, while immunotherapies that reduce or suppress are classified as suppression immunotherapies.

Non-limiting examples of immunotherapies that may be used in the present invention include, allergy shots and oral tolerance therapies.

In certain embodiments, the one or more additional therapeutic agent(s) is a decongestant. Decongestants are a type of agent that is used to relieve nasal congestion in the upper respiratory tract. Non-limiting examples of decongestants that may be used in the present invention include pseudoephedrine, phenylephrine, and oxymetazoline.

In certain embodiments, the one or more additional therapeutic agent(s) is a bronchodilator. Bronchodilators are a type of agent that dilates the bronchi and bronchioles, decreasing resistance in the respiratory airway and increasing airflow to the lungs. Non-limiting examples of bronchodilators that may be used in the present invention include albuterol and levalbuterol.

In certain embodiments, the one or more additional therapeutic agent(s) is a mast cell stabilizer. Mast cell stabilizers are generally cromone medications that are used to prevent or control certain allergic disorders. They block a calcium channel essential for mast cell degranulation, stabilizing the cell and thereby preventing the release of histamine and related mediators. Non-limiting examples of mast cell stabilizers that may be used in the present invention include cromolyn sodium, lodoxamide, and nedocromil.

In certain embodiments, the one or more additional therapeutic agent(s) is a leukotriene modifier. Leukotriene modifiers are a type of agent that functions as a leukotriene-related enzyme inhibitor (arachidonate 5-lipoxygenase) or leukotriene receptor antagonist (cysteinyl leukotriene receptors), and consequently, opposes the function of these inflammatory mediators. Non-limiting examples of leukotriene modifiers that may be used in the present invention include monteleukast, zafirlukast, and zyflo.

In certain embodiments, the one or more additional therapeutic agent(s) is a tyrosine kinase inhibitor (TKIs), such as KIT inhibitors. Tyrosine kinases are enzymes that activate signal transduction cascades by phosphorylating proteins involved in those signaling processes. TKIs inhibit tyrosine kinase phosphorylation. Non-limiting examples of TKIs that may be used in the present invention include imatinib, sunitinib, dasatinib, nilotinib, avapritinib, and bezuclatinib.

### Administration and Formulation

The compounds of Formula I or Formula A can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of Formula I or Formula A to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of Formula I or Formula A can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents. Examples of such agents include anti-histamines, steroids, immunotherapies (e.g., allergy shots, oral tolerance therapies, etc.), decongestants, bronchodilators, mast cell stabilizers, leukotriene modifiers, prostaglandin antagonists, and blocking/neutralizing antibodies (e.g., anti-IgE therapies, anti-IL-4R alpha therapies, and anti-SIGLEC8 therapies).

Accordingly, one embodiment the invention also provides a composition comprising a compound of Formula I or Formula A, or a pharmaceutically acceptable salt thereof, at least one other therapeutic agent (e.g., an additional agent described herein), and a pharmaceutically acceptable diluent or carrier. The invention also provides a kit comprising a compound of Formula I or Formula A, or a pharmaceutically acceptable salt thereof, at least one other therapeutic agent, packaging material, and instructions for administering the compound of Formula I or Formula A or the pharmaceutically acceptable salt thereof and the other therapeutic agent or agents to an animal to treat a disease or condition that is mediated by carbonic anhydrase.

### Non-Limiting Exemplary Embodiments

In some embodiments, the present disclosure provides the following non-limiting exemplary enumerated Embodiments 1-48:
Embodiment 1. A compound of Formula I:

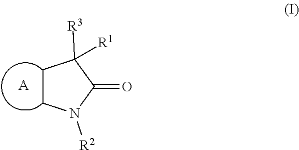

or a pharmaceutically acceptable salt thereof, wherein:
   R¹ is aryl, 5-membered heteroaryl, 6-membered heteroaryl, or (C₁-C₃)alkyl that is substituted with aryl, 5-membered heteroaryl, or 6-membered heteroaryl, wherein any aryl, 5-membered heteroaryl, and 6-membered heteroaryl, is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;
   R² is H, aryl, 5-membered heteroaryl, 6-membered heteroaryl, or (C₁-C₃)alkyl that is optionally substituted with aryl, 5-membered heteroaryl, or 6-membered heteroaryl, wherein any aryl, 5-membered heteroaryl, and 6-membered heteroaryl, is optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{c}R^{d}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;
   R³ is H, fluoro, hydroxy, (C₁-C₆)alkyl, or (C₁-C₆)alkoxy, wherein any (C₁-C₆)alkyl and (C₁-C₆)alkoxy, is optionally substituted with one or more fluoro;
   ring A is phenyl, 5-membered heteroaryl, or 6-membered heteroaryl, and ring A is optionally substituted with 1, 2, 3, or 4 groups, as valency permits, independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;
   each R^{a} and R^{b} is independently selected from the group consisting of H, (C₁-C₆)alkyl, (C₃-C₆)cycloalkyl, and (C₃-C₆)cycloalkyl(C₁-C₆)alkyl; or R^{a} and R^{b} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl;
   each R^{c} and R^{d} is independently selected from the group consisting of H, (C₁-C₆)alkyl, (C₃-C₆)cycloalkyl, and (C₃-C₆)cycloalkyl(C₁-C₆)alkyl; or R^{c} and R^{d} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl; and
   each R^{e} and R^{f} is independently selected from the group consisting of H, (C₁-C₆)alkyl, (C₃-C₆)cycloalkyl, and (C₃-C₆)cycloalkyl(C₁-C₆)alkyl; or R and R together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl.
Embodiment 2. The compound or pharmaceutically acceptable salt of Embodiment 1, wherein the compound of Formula I is a compound of Formula Ia:

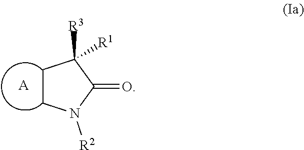

Embodiment 3. The compound or pharmaceutically acceptable salt of Embodiment 1, wherein the compound of Formula I is a compound of Formula Ib:

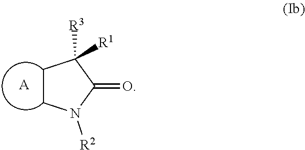

Embodiment 4. The compound or pharmaceutically acceptable salt of any one of Embodiments 1-3, wherein R¹ is aryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 5. The compound or pharmaceutically acceptable salt of any one of Embodiments 1-3, wherein R¹ is 5-membered heteroaryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 6. The compound or pharmaceutically acceptable salt of any one of Embodiments 1-3, wherein R¹ is 6-membered heteroaryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 7. The compound or pharmaceutically acceptable salt of any one of Embodiments 1-3, wherein R¹ is (C₁-C₃)alkyl that is substituted with aryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 8. The compound or pharmaceutically acceptable salt of any one of Embodiments 1-3, wherein R¹ is (C₁-C₃)alkyl that is substituted with 5-membered heteroaryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 9. The compound or pharmaceutically acceptable salt of any one of Embodiments 1-3, wherein R¹ is (C₁-C₃)alkyl that is substituted with 6-membered heteroaryl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 10. The compound or pharmaceutically acceptable salt of any one of Embodiments 1-3, wherein R¹ is phenyl that is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 11. The compound or pharmaceutically acceptable salt of any one of Embodiments 1-3, wherein R¹ is phenyl that is substituted with —S(═O)₂NH₂.
Embodiment 12. The compound or pharmaceutically acceptable salt of any one of Embodiments 1-3, wherein R¹ is 4-(aminosulfonyl)phenyl.
Embodiment 13. The compound or pharmaceutically acceptable salt of any one of Embodiments 1-12, wherein R² is benzyl that is optionally substituted with one or more groups independently selected from the group consisting of halo, hydroxy, cyano, nitro, (C₁-C₆)alkyl, (C₁-C₆)alkoxy, (C₁-C₆)alkanoyl, (C₁-C₆)alkoxycarbonyl, (C₁-C₆)alkanoyloxy, and NR^{c}R^{d}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 14. The compound or pharmaceutically acceptable salt of any one of Embodiments 1-12, wherein R² is benzyl that is optionally substituted with (C₁-C₆)alkoxy.
Embodiment 15. The compound or pharmaceutically acceptable salt of any one of Embodiments 1-12, wherein R² is 4-methoxybenzyl.
Embodiment 16. The compound or pharmaceutically acceptable salt of any one of Embodiments 1-15, wherein R³ is H.
Embodiment 17. The compound or pharmaceutically acceptable salt of any one of Embodiments 1-15, wherein R³ is fluoro.
Embodiment 18. The compound or pharmaceutically acceptable salt of any one of Embodiments 1-15, wherein R³ is (C₁-C₆)alkyl that is optionally substituted with one or more fluoro.
Embodiment 19. The compound or pharmaceutically acceptable salt of any one of Embodiments 1-15, wherein R³ is (C₁-C₆)alkoxy that is optionally substituted with one or more fluoro.
Embodiment 20. The compound or pharmaceutically acceptable salt of any one of Embodiments 1-19, wherein ring A is phenyl, and ring A is optionally substituted with 1, 2, 3, or 4 groups independently selected from the group consisting of halo, hydroxy, cyano, nitro, (C₁-C₆)alkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 21. The compound or pharmaceutically acceptable salt of any one of Embodiments 1-19, wherein ring A is 5-membered heteroaryl, and ring A is optionally substituted with 1, 2, 3, or 4 groups independently selected from the group consisting of halo, hydroxy, cyano, nitro, (C₁-C₆)alkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 22. The compound or pharmaceutically acceptable salt of any one of Embodiments 1-19, wherein ring A is 6-membered heteroaryl, and ring A is optionally substituted with 1, 2, 3, or 4 groups independently selected from the group consisting of halo, hydroxy, cyano, nitro, (C₁-C₆)alkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy.
Embodiment 23. The compound or pharmaceutically acceptable salt of Embodiment 1, wherein the compound of Formula I is a compound of Formula IIa:

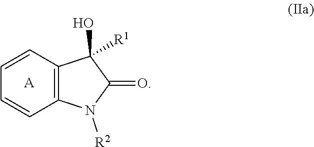

Embodiment 24. The compound or pharmaceutically acceptable salt of Embodiment 1, wherein the compound of Formula I is a compound of Formula IIb:

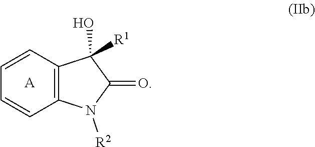

Embodiment 25. The compound or pharmaceutically acceptable salt of Embodiment 1, wherein the compound of Formula I is a compound of Formula IIIa:

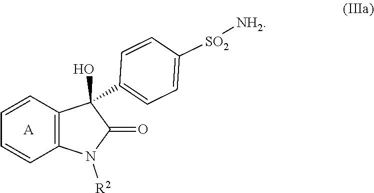

Embodiment 26. The compound or pharmaceutically acceptable salt of Embodiment 1, wherein the compound of Formula I is a compound of Formula IIIb:

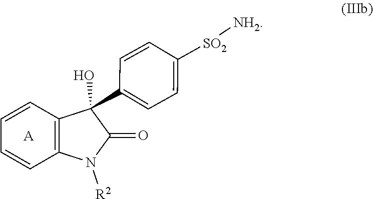

Embodiment 27. The compound:

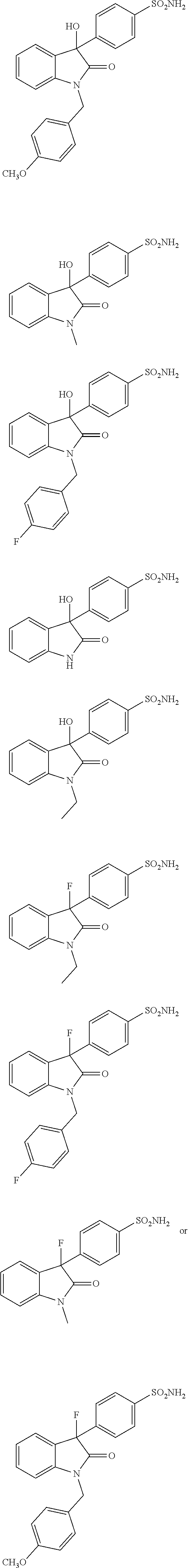

or a pharmaceutically acceptable salt thereof
Embodiment 28. A pharmaceutical composition comprising a compound as described in any one of Embodiments 1-27 or a pharmaceutically acceptable salt thereof and a pharmaceutically acceptable excipient.
Embodiment 29. A compound as described in any one of Embodiments 1-27 or a pharmaceutically acceptable salt thereof, for use in medical therapy.
Embodiment 30. A method of inhibiting a carbonic anhydrase enzyme in vitro or in vivo comprising contacting the carbonic anhydrase enzyme with an effective amount of a compound as described in any one of Embodiments 1-27 or a pharmaceutically acceptable salt thereof.
Embodiment 31. A method of treating a disease or condition mediated by a carbonic anhydrase enzyme in a mammal (e.g., a human), comprising administering a compound as described in any one of Embodiments 1-27, or a pharmaceutically acceptable salt thereof, to the mammal.
Embodiment 32. The method Embodiment 31, further comprising administering one or more additional therapeutic agent(s).
Embodiment 33. The method of Embodiment 32, wherein the one or more additional therapeutic agent(s) is an anti-histamine, a steroid, a decongestant, a bronchodilator, a mast cell stabilizer, a leukotriene modifier, a prostaglandin antagonist, a blocking/neutralizing antibody, and/or an immunotherapy.
Embodiment 34. The method of Embodiment 32, wherein the one or more additional therapeutic agent(s) is an anti-histamine.
Embodiment 35. The method of Embodiment 34, wherein the anti-histamine is acrivastine, azelastine, bilastine, brompheniramine, buclizine, bromodiphenhydramine, carbinoxamine, cetirizine (Zyrtec; metabolite of hydroxyzine, its prodrug), chlorpromazine, cimetidine, cyclizine, chlorphenamine, chlorodiphenhydramine, clemastine, cyproheptadine, desloratadine, dexbrompheniramine, dexchlorpheniramine, dimetindene, diphenhydramine (Benadryl), ebastine, embramine, famotidine, fexofenadine (Allegra), hydroxyzine (Vistaril), lafutidine, levocetirizine, loratadine (Claritin), nizatidine, olopatadine, phenindamine, pheniramine, phenyltoloxamine, promethazine, pyrilamine, ranitidine, roxatidine, rupatadine, tiotidine, tripelennamine, or triprolidine.
Embodiment 36. The method of any one of Embodiments 30-35, wherein the mammal is a human, mouse, rat, dog, cat, hamster, guinea pig, rabbit or livestock.
Embodiment 37. A compound as described in any one of Embodiments 1-27 or a pharmaceutically acceptable salt thereof, for the prophylactic or therapeutic treatment of a disease or condition mediated by a carbonic anhydrase enzyme.
Embodiment 38. The use of a compound as described in any one of Embodiments 1-27 or a pharmaceutically acceptable salt thereof, to prepare a medicament for treating a disease or condition mediated by a carbonic anhydrase enzyme.
Embodiment 39. The method, compound or use of any one of Embodiments 30-38, wherein the disease or condition mediated by a carbonic anhydrase enzyme is an allergic disease, a bacterial infection, a fungal infection, a viral infection, mastocytosis or mast cell-mediated inflammation.
Embodiment 40. The method, compound or use of Embodiment 39, wherein the disease or condition mediated by a carbonic anhydrase enzyme is an allergic disease.
Embodiment 41. The method, compound or use of Embodiment 40, wherein the allergic disease is asthma (e.g., fungal asthma), atopic dermatitis, contact dermatitis, chronic itch (pruritus), urticaria, hay fever, allergic conjunctivitis, allergic rhinitis, anaphylaxis, eosinophilic esophagitis, a food allergy(ies) or allergen-induced mastocytosis.
Embodiment 42. The method, compound or use of Embodiment 40, wherein the allergic disease is asthma.
Embodiment 43. The method, compound or use of Embodiment 40, wherein the allergic disease is a food allergy.
Embodiment 44. The method, compound or use of Embodiment 39, wherein the disease or condition mediated by a carbonic anhydrase enzyme is a bacterial infection.
Embodiment 45. The method, compound or use of Embodiment 39, wherein the disease or condition mediated by a carbonic anhydrase enzyme is a fungal infection.
Embodiment 46. The method, compound or use of Embodiment 39, wherein the disease or condition mediated by a carbonic anhydrase enzyme is a viral infection.
Embodiment 47. The method, compound or use of Embodiment 39, wherein the disease or condition mediated by a carbonic anhydrase enzyme is mastocytosis.
Embodiment 48. The method, compound or use of Embodiment 39, wherein the disease or condition mediated by a carbonic anhydrase enzyme is mast cell-mediated inflammation.

The invention will now be illustrated by the following non-limiting Examples.

### EXAMPLES

Mast cells are potent innate immune cells that activate in response to diverse stimuli, including cytokines and antigen-antibody complexes. Upon activating, mast cells release numerous effector molecules that are well described for their ability to promote detrimental inflammation in the context of allergies, asthma, mastocytosis and mast cell activation syndrome. Despite the clinical need, therapeutic strategies to target mast cell responses and reduce mast cell activation remain limited.

Mast cell progenitors that express high levels of the enzyme Carbonic anhydrase (Car)1 were recently identified. Using these cells, it was demonstrated that targeting Car1 with the inhibitor methazolamide (MZ) was sufficient to prevent mast cell development and mast cell-mediated inflammation. As described below, a more potent Car1 inhibitor was designed and synthesized (4-(3-hydroxy-1-(4-methoxybenzyl)-2-oxoindolin-3-yl)benzenesulfonamide (CAR0037)). In particular, it was shown that CAR0037 is a more potent inhibitor of Car1 and more efficiently inhibits mast cell responses as compared to MZ. Additional studies show that other compounds of the present disclosure can also have similar or better efficacy in inhibiting carbonic anhydrase than MZ and/or CAR0037. In summary, these data suggest that the compounds of Formula I or Formula A described herein, including CAR0037, may be used for the treatment of mast cell-mediated inflammation and other mast-cell mediated diseases or conditions.

The abbreviations used in the Examples section should be understood as having their ordinary meanings in the art unless specifically indicated otherwise or obviously contrary from context.

### Example 1. Preparation of 4-(3-hydroxy-1-(4-methoxybenzyl)-2-oxoindolin-3-yl)benzenesulfonamide

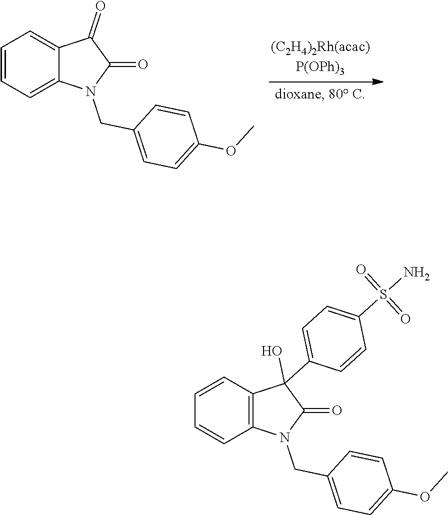 [SMILES: COc1ccc(CN2C(=O)C(=O)c3ccccc32)cc1 ; COc1ccc(CN2C(=O)C(O)(c3ccc(S(N)(=O)=O)cc3)c3ccccc32)cc1]

Acetylacetonatobis(ethylene)rhodium(I) (13.2 mg, 0.051 mmol, 3 mol %), and triphenyl phosphite (36.9 mg, 0.12 mmol, 7 mol %) in dry dioxane were degassed under bubbling nitrogen for 10 minutes. To this solution was added a mixture of 1-(4-methoxybenzyl)indoline-2,3-dione (457 mg, 1.7 mmol, 1.0 equiv.) and (4-sulfamoylphenyl)boronic acid (687 mg, 3.4 mmol, 2.0 equiv.) in dry dioxane. The reaction was heated overnight at 80° C. After the reaction was complete (as monitored by TLC), the reaction mixture was extracted with ethyl acetate (3×5 mL) and washed with water (2×10 mL). The ethyl acetate layer was separated and dried over Na₂SO₄. After evaporation of the solvent, the residue was purified by flash column chromatography (ethyl acetate/hexane) to give the title compound (210 mg, 29.1%) as a white solid. ¹H NMR (CDCl₃, 500 MHz): δ 7.76 (2H, d, J=8.5 Hz); 7.44 (2H, d, J=8.5); 7.27-7.23 (3H, m); 7.18 (1H, d, J=7.0 Hz); 7.05-7.02 (1H, m); 6.87 (3H, d, J=8.5 Hz); 5.29 (2H, d, J=2.5 Hz); 4.95 (1H, d, 15.5 Hz); 4.78 (1H, d, J=15.5 Hz); 4.52 (1H, s), 3.78 (3H, s). LCMS: 425.2 [M+H]⁺.

The intermediate compound 1-(4-methoxybenzyl)indoline-2,3-dione was prepared as follows.

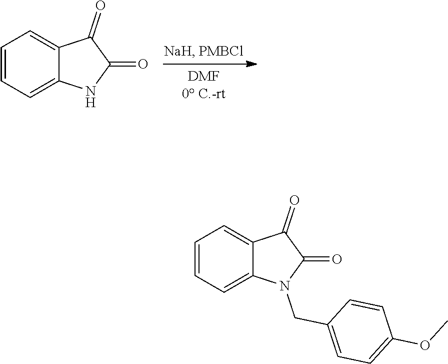 [SMILES: O=C1Nc2ccccc2C1=O ; COc1ccc(CN2C(=O)C(=O)c3ccccc32)cc1]

### a. 1-(4-methoxybenzyl)indoline-2,3-dione

To a solution of isatin (500 mg, 3.4 mmol, 1.0 equiv.) in anhydrous DMF under nitrogen at 0° C. was added NaH (60% in mineral oil) (148 mg, 3.7 mmol, 1.1 equiv.) over a 15 min. period. After 30 min., 4-methoxybenzyl chloride (509 μL, 3.7 mmol, 1.1 equiv.) was added dropwise and the reaction was stirred for another 8 h. At completion, the solution was concentrated under reduced pressure and redissolved in EtOAc. This solution was washed with water, brine, dried over anhydrous Na₂SO₄, filtered and concentrated. The crude product was purified by recrystallization in hot EtOAc/hexanes to give 1-(4-methoxybenzyl)indoline-2,3-dione (410 mg, 45%) as an orange solid. ¹H NMR (CDCl₃, 500 MHz): δ 7.57 (1H, dd, J=0.8, 6.8 Hz); 7.47 (1H, dt, J=1.2, 7.6 Hz), 7.22-7.29 (2H, m), 7.06 (1H, dt, J=0.4, 7.6 Hz), 6.82-6.88 (2H, m), 6.79 (1H, d, J=8.0 Hz), 4.85 (2H, s), 3.77 (3H, s). LCMS: 289.7 [M+Na]⁺.

### Example 2. Alternative Preparation of 4-(3-hydroxy-1-(4-methoxybenzyl)-2-oxoindolin-3-yl)benzenesulfonamide

A mixture of added PMB-isatin (0.25 mmol, 1.0 equiv), arylboronic acid (0.28 mmol, 1.1 equiv), Rh₂(OAc)₄ (0.003 mmol, 1 mol %), [(t-Bu)₃PH]BF₄ (0.0075 mmol, 2.5 mol %), anhydrous K₂CO₃ (0.015 mmol, 5 mol %) was evacuated and purged three times with nitrogen. DME/H₂O (1 mL, 1/1 v/v) was added, and the mixture was stirred at 90° C. for 12 h. After the reaction was complete (as monitored by TLC), the reaction mixture was extracted with ethyl acetate (3×5 mL) and washed with water (2×10 mL). The ethyl acetate layer was separated and dried over Na₂SO₄. After evaporation of the solvent, the residue was purified by flash column chromatography (ethyl acetate/hexane) to give the title compound.

¹H NMR (CDCl₃, 500 MHz): δ 7.76 (2H, d, J=8.5 Hz); 7.44 (2H, d, J=8.5); 7.27-7.23 (3H, m); 7.18 (1H, d, J=7.0 Hz); 7.05-7.02 (1H, m); 6.87 (3H, d, J=8.5 Hz); 5.29 (2H, d, J=2.5 Hz); 4.95 (1H, d, 15.5 Hz); 4.78 (1H, d, J=15.5 Hz); 4.52 (1H, s), 3.78 (3H, s).

### Example 3. Preparation of 4-(3-fluoro-1-(4-methoxybenzyl)-2-oxoindolin-3-yl)benzenesulfonamide

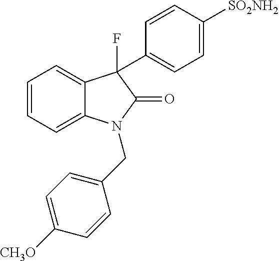

To a solution of 4-(3-hydroxy-1-(4-methoxybenzyl)-2-oxoindolin-3-yl)benzenesulfonamide (CAR-0037) (100 mg, 0.24 mmol, 1 eq) in DCM at 0° C. was added DAST (37 μL, 0.28 mmol, 1.2 eq). The reaction was slowly warmed to room temperature and stirred for 2 hours. At completion, the reaction was diluted in DCM and quenched with NaHCO₃ (aq, sat). The organic was separated, dried over Na₂SO₄, filtered and concentrated. Purification by silica gel chromatography (Hex/EtOAc) afforded the title compound as a white solid (78 mg, 76.2%). LCMS: 427.2 [M+H]⁺.

### Example 4. Preparation of 4-(3-hydroxy-1-methyl-2-oxoindolin-3-yl)benzenesulfonamide

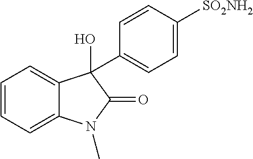 [SMILES: CN1C(=O)C(O)(c2ccc(S(N)(=O)=O)cc2)c2ccccc21]

Using a procedure similar to that described in Example 1, except using 1-methylindoline-2,3-dione (161 mg, 1 mmol) and (4-sulfamoylphenyl)boronic acid (402 mg, 2.0 mmol, 2 equiv.), the title compound was prepared (160 mg, 50%) as a beige solid. LCMS: 319.2 [M+H]⁺.

### Example 5. Preparation of 4-(3-fluoro-1-methyl-2-oxoindolin-3-yl)benzenesulfonamide

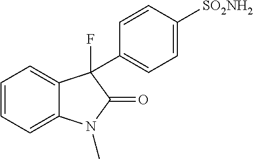 [SMILES: CN1C(=O)C(F)(c2ccc(S(N)(=O)=O)cc2)c2ccccc21]

Using a procedure similar to that described in Example 3, except using 4-(3-hydroxy-1-methyl-2-oxoindolin-3-yl)benzenesulfonamide (66 mg, 0.21 mmol), the title compound was prepared (42 mg) as a white solid. LCMS: 321.2 [M+H]⁺.

### Example 6. Preparation of 4-(1-(4-fluorobenzyl)-3-hydroxy-2-oxoindolin-3-yl)benzenesulfonamide

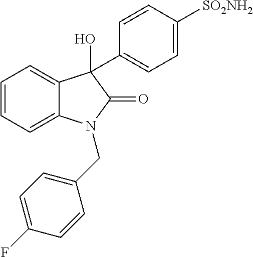 [SMILES: NS(=O)(=O)c1ccc(C2(O)C(=O)N(Cc3ccc(F)cc3)c3ccccc32)cc1]

Using a procedure similar to that described in Example 1, except using 1-(4-fluorobenzyl)indoline-2,3-dione (174 mg, 0.68 mmol) and (4-sulfamoylphenyl)boronic acid (273 mg, 1.36 mmol, 2 equiv.) the title compound was prepared (102 mg, 36%) as a white solid. LCMS: 413.2 [M+H]⁺.

The intermediate compound 1-(4-fluorobenzyl)indoline-2,3-dione was prepared as follows.

### a. 1-(4-fluorobenzyl)indoline-2,3-dione

Using a procedure similar to that described in Example 1, sub-part a, and using indoline-2,3-dione (588 mg, 4 mmol) and 4-fluorobenzyl chloride (636 mg, 4.4 mmol, 1.1 equiv.) the title compound was prepared (708 mg, 69%) as an orange solid. LCMS: 255.5 [M+H]⁺.

### Example 7. Preparation of 4-(3-fluoro-1-(4-fluorobenzyl)-2-oxoindolin-3-yl)benzenesulfonamide

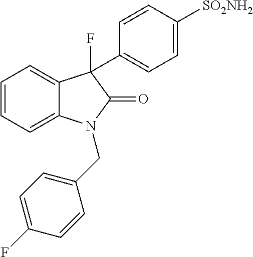 [SMILES: NS(=O)(=O)c1ccc(C2(F)C(=O)N(Cc3ccc(F)cc3)c3ccccc32)cc1]

Using a procedure similar to that described in Example 3, except using 4-(1-(4-fluorobenzyl)-3-hydroxy-2-oxoindolin-3-yl)benzenesulfonamide (40 mg, 0.10 mmol) the title compound was prepared (29 mg, 72%) as a white solid. LCMS: 415.22 [M+H]⁺.

### Example 8. Preparation of 4-(3-hydroxy-2-oxoindolin-3-yl)benzenesulfonamide

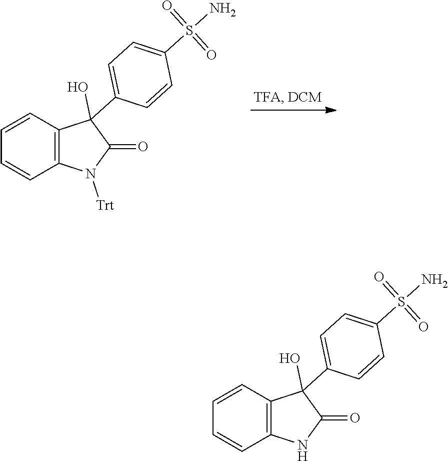

4-(3-hydroxy-2-oxo-1-tritylindolin-3-yl)benzenesulfonamide (180 mg, 0.33 mmol) was dissolved in a mixture of DCM (10 ml) and TFA (5 ml) and stirred overnight at room temperature. The reaction was determined complete by LCMS and concentrated under reduced pressure. The residue was taken up in DCM and washed 1×NaHCO₃ (sat, aq), 1×water, 1×brine). The organic was dried over sodium sulfate and concentrated to afford the title compound as a beige solid. LCMS: 305.1 [M+H]⁺.

The intermediate compound 4-(3-hydroxy-2-oxo-1-tritylindolin-3-yl)benzenesulfonamide was prepared as follows.

### a. 1-tritylindoline-2,3-dione

Using a procedure similar to that described in Example 1, sub-part a, and using indoline-2,3-dione (500 mg, 3.4 mmol) and trityl chloride (1.04 g, 3.74 mmol, 1.1 equiv.) the title compound was prepared (720 mg, 55%) as a yellow/orange solid. LCMS: 411.9 [M+Na]⁺.

### b. Preparation of 4-(3-hydroxy-2-oxo-1-tritylindolin-3-yl)benzenesulfonamide

Using a procedure similar to that described in Example 1, except using 1-tritylindoline-2,3-dione (530 mg, 1.36 mmol) and (4-sulfamoylphenyl)boronic acid (547 mg, 2.72 mmol, 2 equiv.) the title compound was prepared (220 mg, 29.6%) as a yellow solid. LCMS: 568.7 [M+Na]⁺.

### Example 9. Preparation of 4-(1-ethyl-3-hydroxy-2-oxoindolin-3-yl)benzenesulfonamide

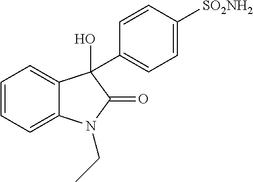 [SMILES: CCN1C(=O)C(O)(c2ccc(S(N)(=O)=O)cc2)c2ccccc21]

Using a procedure similar to that described in Example 1, except using 1-ethylindoline-2,3-dione (176 mg, 1.0 mmol) and (4-sulfamoylphenyl)boronic acid (402 mg, 2.0 mmol, 2 equiv.) the title compound was prepared (204 mg, 61.4%) as a white solid. LCMS: 333.2 [M+H]⁺.

The intermediate compound 1-ethylindoline-2,3-dione was prepared as follows.

### a. 1-ethylindoline-2,3-dione

Using a procedure similar to that described in Example 1, sub-part a, and using indoline-2,3-dione (1.47 g, 10 mmol, 1 equiv.) and bromoethane (0.76 ml, 10 mmol, 1 equiv.) to give the title compound was prepared (1.57 g, 90%) as a bright orange solid. LCMS: 176.3 [M+H]⁺.

### Example 10. Preparation of 4-(1-ethyl-3-fluoro-2-oxoindolin-3-yl)benzenesulfonamide

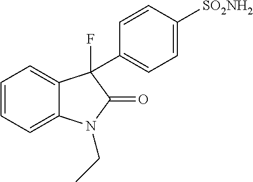 [SMILES: CCN1C(=O)C(F)(c2ccc(S(N)(=O)=O)cc2)c2ccccc21]

Using a procedure similar to that described in Example 3, except using 4-(1-ethyl-3-hydroxy-2-oxoindolin-3-yl)benzenesulfonamide (90 mg, 0.27 mmol) the title compound was prepared (57 mg, 63%) as a white solid. LCMS: 335.1 [M+H]⁺.

### Example 11. Synthesis of Compound NEM-4A

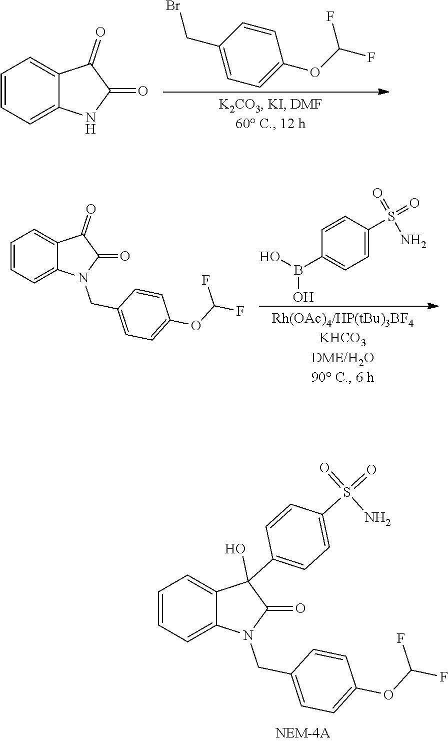 [SMILES: O=C1Nc2ccccc2C1=O ; FC(F)Oc1ccc(CBr)cc1 ; O=C1C(=O)N(Cc2ccc(OC(F)F)cc2)c2ccccc21 ; NS(=O)(=O)c1ccc(B(O)O)cc1 ; NS(=O)(=O)c1ccc(C2(O)C(=O)N(Cc3ccc(OC(F)F)cc3)c3ccccc32)cc1]

### 1. Synthesis of 1-[[4-(difluoromethoxy)phenyl]methyl]indoline-2,3-dione

A mixture of indoline-2,3-dione (300 mg, 2.04 mmol, 1 eq), 1-(bromomethyl)-4-(difluoromethoxy)benzene (483.33 mg, 2.04 mmol, 1 eq), KI (135.39 mg, 815.60 umol, 0.4 eq), K₂CO₃ (563.61 mg, 4.08 mmol, 2 eq) in DMF (5 mL) was degassed and purged with N₂ for 3 times, and then the mixture was stirred at 60° C. for 3 h under N₂ atmosphere. The reaction mixture was cooled to room temperature and diluted by water (15 mL), extracted with ethyl acetate (20 mL*2), the combined organics were washed with brine (15 mL), dried over Na₂SO₄, filtered and concentrated under reduced pressure to give a residue. The crude product 1-[[4-(difluoromethoxy)phenyl]methyl]indoline-2,3-dione (350 mg, 1.15 mmol) was obtained as a red solid which was used into the next step without further purification. LCMS (ESI) m/z: 304.1 [M+H]⁺

### 2. Synthesis of 4-[1-[[4-(difluoromethoxy)phenyl]methyl]-3-hydroxy-2-oxo-indolin-3-yl]benzenesulfonamide (NEM-4A)

A mixture of 1-[[4-(difluoromethoxy)phenyl]methyl]indoline-2,3-dione (300 mg, 989.25 umol, 1 eq), (4-sulfamoylphenyl)boronic acid (258.50 mg, 1.29 mmol, 1.3 eq), diacetoxyrhodium (2.19 mg, 9.89 umol, 0.01 eq), K₂CO₃ (6.84 mg, 49.46 umol, 0.05 eq) and tritert-butylphosphonium;tetrafluoroborate (8.61 mg, 29.68 umol, 0.03 eq) in DME (3 mL), H₂O (3 mL) was degassed and purged with N₂ for 3 times, and then the mixture was stirred at 90° C. for 5 h under N₂ atmosphere. The reaction mixture was cooled to room temperature and diluted with water (15 mL), extracted with ethyl acetate (20 mL*2). The residue was purified by preparative HPLC (column: Phenomenex Luna 80*30 mm*3 um;mobile phase: [water(TFA)-ACN]; B %: 30%-60%, 8 min). 4-[1-[[4-(difluoromethoxy)phenyl]methyl]-3-hydroxy-2-oxo-indolin-3-yl]benzenesulfonamide (26.50 mg, 56.57 umol, 5.72%) was obtained as a pale yellow solid.

¹H NMR (400 MHz, ACETONITRILE-d3) δ 7.86-7.82 (m, 2H), 7.54-7.49 (m, 2H), 7.39 (d, J=8.8 Hz, 2H), 7.32 (dt, J=1.3, 7.8 Hz, 1H), 7.21 (dd, J=0.8, 7.4 Hz, 1H), 7.14 (d, J=8.6 Hz, 2H), 7.11-7.06 (m, 1H), 6.96 (d, J=7.9 Hz, 1H), 6.75 (t, J=74.2 Hz, 1H), 5.65 (s, 2H), 4.96-4.86 (m, 2H) LCMS (ESI) m/z: 461.0 [M+H]⁺

### Example 12. Synthesis of Compound NEM-9A

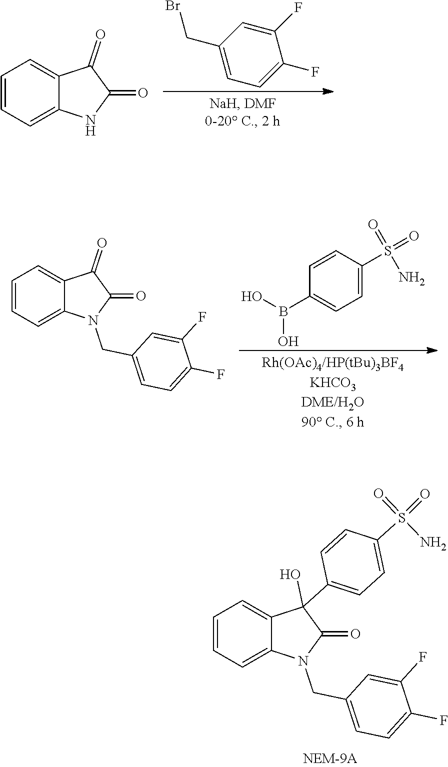 [SMILES: O=C1Nc2ccccc2C1=O ; Fc1ccc(CBr)cc1F ; O=C1C(=O)N(Cc2ccc(F)c(F)c2)c2ccccc21 ; NS(=O)(=O)c1ccc(B(O)O)cc1 ; NS(=O)(=O)c1ccc(C2(O)C(=O)N(Cc3ccc(F)c(F)c3)c3ccccc32)cc1]

### 1. Synthesis of 1-[(3,4-difluorophenyl)methyl]indoline-2,3-dione

NaH (326.21 mg, 8.16 mmol, 60% purity, 1.2 eq) was added to a solution of indoline-2,3-dione (1 g, 6.80 mmol, 1 eq) in DMF (10 mL) at 0° C., the mixture was stirred for 10 min at 0° C., then 4-(bromomethyl)-1,2-difluoro-benzene (1.55 g, 7.48 mmol, 955.38 uL, 1.1 eq) was added. The mixture was stirred for 3 h at 20° C. The mixture poured into 20 mL of ice cold water, the solid was collected by filtration, washed with H₂O (5 mL*3) and dried under reduced pressure to give crude product. The crude product 1-[(3,4-difluorophenyl)methyl]indoline-2,3-dione (900 mg, crude) was obtained as a red solid which was used into the next step without further purification. LCMS (ESI) m/z: 274.2 [M+H]⁺

### 2. Synthesis of 4-[1-[(3,4-difluorophenyl)methyl]-3-hydroxy-2-oxo-indolin-3-yl]benzenesulfonamide (NEM-9A)

A mixture of 1-[(3,4-difluorophenyl)methyl]indoline-2,3-dione (300 mg, 1.10 mmol, 1 eq), (4-sulfamoylphenyl)boronic acid (286.91 mg, 1.43 mmol, 1.3 eq), diacetoxyrhodium (2.43 mg, 10.98 umol, 0.01 eq), K₂CO₃ (7.59 mg, 54.90 umol, 0.05 eq) and tritert-butylphosphonium;tetrafluoroborate (9.56 mg, 32.94 umol, 0.03 eq) in DME (3 mL) and H₂O (3 mL) was degassed and purged with N₂ for 3 times, and then the mixture was stirred at 90° C. for 5 h under N₂ atmosphere. The reaction mixture was cooled to room temperature and diluted by water (15 mL), extracted with ethyl acetate (20 mL*2). The organic layer was washed with brine (5 mL), dried over Na₂SO₄ and concentrated to give crude product. The residue was purified by preparative HPLC (column: Phenomenex Luna 80*30 mm*3 um;mobile phase: [water(TFA)-ACN];B %: 30%-60%, 8 min). Compound 4-[1-[(3,4-difluorophenyl)methyl]-3-hydroxy-2-oxo-indolin-3-yl]benzenesulfonamide (41.72 mg, 93.91 umol, 8.54%) was obtained as a pale yellow solid.

¹H NMR (400 MHz, ACETONITRILE-d3) δ=7.86-7.81 (m, 2H), 7.54-7.49 (m, 2H), 7.33 (dt, J=1.3, 7.8 Hz, 1H), 7.30-7.23 (m, 2H), 7.23-7.20 (m, 1H), 7.16 (ddd, J=1.9, 4.1, 6.2 Hz, 1H), 7.12-7.07 (m, 1H), 6.95 (d, J=7.9 Hz, 1H), 5.65 (s, 2H), 4.96-4.82 (m, 2H). LCMS (ESI) m/z: 431.0 [M+H]⁺

### Example 13. Synthesis of Compound NEM-10A

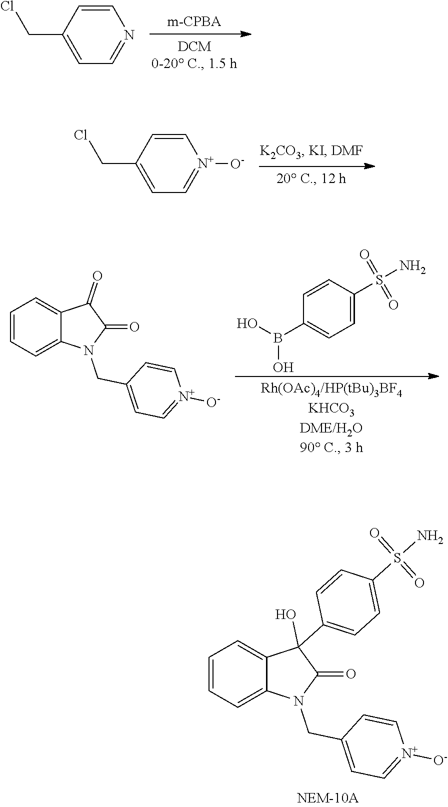 [SMILES: ClCc1ccncc1 ; [O-][n+]1ccc(CCl)cc1 ; O=C1C(=O)N(Cc2cc[n+]([O-])cc2)c2ccccc21 ; NS(=O)(=O)c1ccc(B(O)O)cc1 ; NS(=O)(=O)c1ccc(C2(O)C(=O)N(Cc3cc[n+]([O-])cc3)c3ccccc32)cc1]

### 1. Synthesis of 4-(chloromethyl)-1-oxido-pyridin-1-ium

To a solution of 4-(chloromethyl)pyridine (1.2 g, 9.41 mmol, 1.5 eq) in DCM (15 mL) was added m-CPBA (2.71 g, 12.54 mmol, 80% purity, 2 eq) at 0° C. The mixture was stirred at 20° C. for 12 h. The mixture was treated with aqueous Na₂S₂O₃ (10 mL, 8M in H₂O) and extracted with DCM (3×10 mL). The crude product 4-(chloromethyl)-1-oxido-pyridin-1-ium (600 mg, crude) was obtained as a black brown solid which was used into the next step without further purification.

### 2. Synthesis of 1-[(1-oxidopyridin-1-ium-4-yl)methyl]indoline-2,3-dione

A mixture of indoline-2,3-dione (1 g, 6.80 mmol, 1 eq), 4-(chloromethyl)-1-oxido-pyridin-1-ium (975.80 mg, 6.80 mmol, 1 eq), K₂CO₃ (939.34 mg, 6.80 mmol, 1 eq), KI (225.65 mg, 1.36 mmol, 0.2 eq) in DMF (10 mL) was degassed and purged with N₂ for 3 times, and then the mixture was stirred at 20° C. for 6 h under N₂ atmosphere. The mixture was filtered and the filtrate was purified by preparative HPLC (column: Phenomenex luna C18 250*50 mm*10 um;mobile phase: [water(TFA)-ACN];B %: 1%-40%, 10 min). Compound 1-[(1-oxidopyridin-1-ium-4-yl)methyl]indoline-2,3-dione (300 mg, 884.99 umol, 13.02%, 75% purity) was obtained as a brown solid. LCMS (ESI) m/z: 255.2 [M+H]⁺,

### 3. Synthesis of 4-[3-hydroxy-1-[(1-oxidopyridin-1-ium-4-yl)methyl]-2-oxo-indolin-3-yl]benzenesulfonamide (NME-10A)

A mixture of 1-[(1-oxidopyridin-1-ium-4-yl)methyl]indoline-2,3-dione (250 mg, 983.32 umol, 1 eq), (4-sulfamoylphenyl)boronic acid (395.31 mg, 1.97 mmol, 2 eq), diacetoxyrhodium (2.17 mg, 9.83 umol, 0.01 eq), tritert-butylphosphonium;tetrafluoroborate (8.56 mg, 29.50 umol, 0.03 eq) and K₂CO₃ (6.80 mg, 49.17 umol, 0.05 eq) in DME (3 mL) and H₂O (3 mL) was degassed and purged with N₂ for 3 times, and then the mixture was stirred at 90° C. for 6 h under N₂ atmosphere. The reaction mixture was partitioned between EtOAc (30 mL) and H₂O (20 mL). The organic phase was separated, washed with aqueous NaCl 20 mL, dried over Na₂SO₄, filtered and concentrated under reduced pressure to give a residue. The residue was purified by preparative HPLC (column: Waters Xbridge Prep OBD C18 150*40 mm*10 um;mobile phase: [water(NH4HCO3)-ACN];B %: 1%-30%, 8 min). Compound 4-[3-hydroxy-1-[(1-oxidopyridin-1-ium-4-yl)methyl]-2-oxo-indolin-3-yl]benzenesulfonamide (16.76 mg, 39.26 umol, 3.99%, 96.37% purity) was obtained as a pale yellow solid.

¹H NMR (400 MHz, METHANOL-d4) δ 8.29 (d, J=7.1 Hz, 2H), 7.91-7.85 (m, 2H), 7.58-7.51 (m, 4H), 7.37 (dt, J=1.3, 7.8 Hz, 1H), 7.26 (d, J=6.6 Hz, 1H), 7.18-7.13 (m, 1H), 7.02 (d, J=7.9 Hz, 1H), 5.09-5.00 (m, 2H) LCMS (ESI) m/z: 412.1 [M+H]⁺

### Example 14. Synthesis of Compound NEM-12A

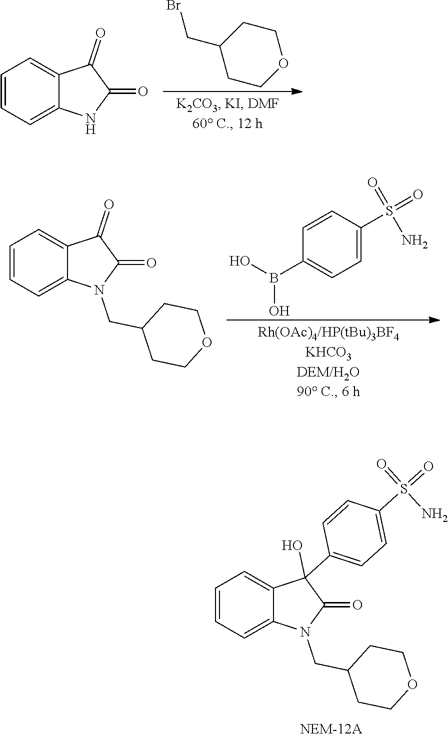 [SMILES: O=C1Nc2ccccc2C1=O ; BrCC1CCOCC1 ; O=C1C(=O)N(CC2CCOCC2)c2ccccc21 ; NS(=O)(=O)c1ccc(B(O)O)cc1 ; NS(=O)(=O)c1ccc(C2(O)C(=O)N(CC3CCOCC3)c3ccccc32)cc1]

### 1. Synthesis of 1-[(4-methoxyphenyl)methyl]indoline-2,3-dione

A mixture of indoline-2,3-dione (300 mg, 2.04 mmol, 1 eq), 4-(bromomethyl)tetrahydropyran (365.09 mg, 2.04 mmol, 1 eq), KI (135.39 mg, 815.60 umol, 0.4 eq), K₂CO₃ (563.61 mg, 4.08 mmol, 2 eq) in DMF (5 mL) was degassed and purged with N₂ for 3 times, and then the mixture was stirred at 60° C. for 3 hr under N₂ atmosphere. The reaction mixture was cooled to room temperature and diluted by water (15 mL), extracted with ethyl acetate (20 mL*2), the combined organics were washed with brine (15 mL), dried over Na₂SO₄, filtered and concentrated under reduced pressure to give a residue. The crude product 1-(tetrahydropyran-4-ylmethyl)indoline-2,3-dione (300 mg, crude) was obtained as a yellow solid which was used into the next step without further purification. LCMS (ESI) m/z: 246.1 [M+H]⁺

### 2. Synthesis of 4-[3-hydroxy-1-[(4-methoxyphenyl)methyl]-2-oxo-indolin-3-yl]benzenesulfonamide (NEM-12A)

A mixture of 1-(tetrahydropyran-4-ylmethyl)indoline-2,3-dione (300 mg, 1.22 mmol, 1 eq), (4-sulfamoylphenyl)boronic acid (319.62 mg, 1.59 mmol, 1.3 eq), diacetoxyrhodium (2.70 mg, 12.23 umol, 0.01 eq), K₂CO₃ (8.45 mg, 61.16 umol, 0.05 eq) and tritert-butylphosphonium;tetrafluoroborate (10.65 mg, 36.69 umol, 0.03 eq) in DME (2 mL) and H₂O (2 mL) was degassed and purged with N₂ for 3 times, and then the mixture was stirred at 90° C. for 5 hr under N₂ atmosphere. The reaction mixture was cooled to room temperature and diluted by water (15 mL), extracted with ethyl acetate (20 mL*2). The organic layer was washed with brine (5 mL), dried over Na₂SO₄ and concentrated to give crude product. The crude product was purified by preparative HPLC (column: Phenomenex Luna 80*30 mm*3 um;mobile phase: [water(TFA)-ACN];B %: 20%-50%, 8 min). Compound 4-[3-hydroxy-2-oxo-1-(tetrahydropyran-4-ylmethyl)indolin-3-yl]benzenesulfonamide (11.66 mg, 27.93 umol, 2.28%, 96.41% purity) was obtained as a pale yellow solid.

¹H NMR (400 MHz, ACETONITRILE-d3) δ 7.84-7.80 (m, 2H), 7.51-7.47 (m, 2H), 7.40 (dt, J=1.3, 7.8 Hz, 1H), 7.18 (dd, J=0.8, 7.3 Hz, 1H), 7.13-7.06 (m, 2H), 5.64 (s, 2H), 3.87 (dddd, J=2.6, 4.5, 6.7, 8.9 Hz, 2H), 3.67-3.53 (m, 2H), 3.34-3.26 (m, 2H), 2.11-2.02 (m, 1H), 1.66-1.51 (m, 2H), 1.40-1.27 (m, 2H) LCMS (ESI) m/z: 403.0 [M+H]⁺

### Example 15. Synthesis of Compounds NEM-27A and 13A

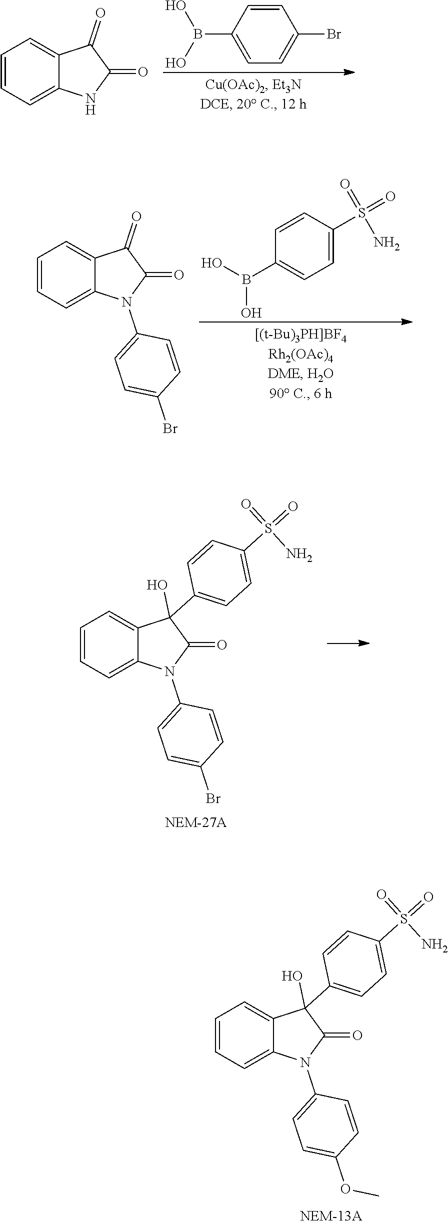

### 1. Synthesis of 1-(4-bromophenyl)indoline-2,3-dione

A mixture of indoline-2,3-dione (2 g, 13.59 mmol, 1 eq), (4-bromophenyl)boronic acid (4.09 g, 20.39 mmol, 1.5 eq), Cu(OAc)₂ (2.47 g, 13.59 mmol, 1 eq), TEA (2.75 g, 27.19 mmol, 3.78 mL, 2 eq) in DCE (10 mL) was degassed and purged with N₂ for 3 times, and then the mixture was stirred at 20° C. for 12 h under N₂ atmosphere. The reaction mixture was diluted with H₂O (50 mL) and extracted with EtOAc 100 mL (50 mL*2). The combined organic layers were washed with brine (20 mL), dried over Na₂SO₄, filtered and concentrated under reduced pressure to give a residue. The residue was purified by flash chromatography on silica gel (Petroleum ether/EtOAc=10:1-1:1). 1-(4-bromophenyl)indoline-2,3-dione (1.5 g, 4.96 mmol, 36.52%) was obtained as a yellow solid. LCMS (ESI) m/z: 302.1 [M+H]⁺

### 2. Synthesis of 4-[1-(4-bromophenyl)-3-hydroxy-2-oxo-indolin-3-yl]benzenesulfonamide (NEM-27A)

A mixture of 1-(4-bromophenyl)indoline-2,3-dione (500 mg, 1.65 mmol, 1 eq), (4-sulfamoylphenyl)boronic acid (665.32 mg, 3.31 mmol, 2 eq), diacetoxyrhodium (7.31 mg, 33.10 umol, 0.02 eq), tritert-butylphosphonium;tetrafluoroborate (19.21 mg, 66.20 umol, 0.04 eq) and K₂CO₃ (22.87 mg, 165.50 umol, 0.1 eq) in DME (4 mL) and H₂O (4 mL) was degassed and purged with N₂ for 3 times, and then the mixture was stirred at 90° C. for 3 h under N₂ atmosphere. The reaction mixture was partitioned between EtOAc (60 mL) and H₂O (30 mL). The organic phase was separated, washed with brine (30 mL), dried over Na₂SO₄, filtered and concentrated under reduced pressure to give a residue. The residue was purified by prep-HPLC (column: Phenomenex luna C18 100*40 mm*3 um;mobile phase: [water(TFA)-ACN];B %: 20%-65%, 8 min. Compound 4-[1-(4-bromophenyl)-3-hydroxy-2-oxo-indolin-3-yl]benzenesulfonamide (200 mg, 435.43 umol, 26.31%, 100% purity) was obtained as a white solid.

¹H NMR (400 MHz, METHANOL-d4) δ 7.89 (d, J=8.5 Hz, 2H), 7.78-7.73 (m, 2H), 7.62 (d, J=8.5 Hz, 2H), 7.44-7.40 (m, 2H), 7.37 (dt, J=1.1, 7.8 Hz, 1H), 7.28 (d, J=6.6 Hz, 1H), 7.20-7.16 (m, 1H), 6.92 (d, J=7.9 Hz, 1H); LCMS (ESI) m/z: 458.9 [M+H]⁺

### 3. Synthesis of 4-[3-hydroxy-1-(4-methoxyphenyl)-2-oxo-indolin-3-yl]benzenesulfonamide (NEM-13A)

A mixture of 4-[1-(4-bromophenyl)-3-hydroxy-2-oxo-indolin-3-yl]benzenesulfonamide (150 mg, 326.58 umol, 1 eq), [2-(2-aminophenyl)phenyl]-methylsulfonyloxy-palladium;ditert-butyl-[2-(2,4,6-triisopropylphenyl)phenyl]phosphane (25.94 mg, 32.66 umol, 0.1 eq), NaOMe (52.93 mg, 979.73 umol, 3 eq) in MeOH (1 mL) was degassed and purged with N₂ for 3 times, and then the mixture was stirred at 70° C. for 12 under N₂ atmosphere. The mixture was added H₂O (3 mL), extracted with EtOAc (10 mL*3). The organic layer was washed with brine (5 mL), dried over Na₂SO₄ and concentrated to give crude product. The crude product was purified by preparative HPLC column: Phenomenex C18 80*30 mm*3 um;mobile phase: [water(TFA)-ACN];B %: 35%-65%, 8 min). Compound 4-[3-hydroxy-1-(4-methoxyphenyl)-2-oxo-indolin-3-yl]benzenesulfonamide (21.4 mg, 51.10 umol, 15.65%, 98% purity) was obtained as a white solid.

¹H NMR (400 MHz, METHANOL-d4) δ 7.90-7.88 (m, 2H), 7.62-7.60 (m, 2H), 7.37-7.34 (m, 3H), 7.26 (dd, J=0.8, 7.4 Hz, 1H), 7.17-7.10 (m, 3H), 6.82-6.80 (d, J=7.8 Hz, 1H), 3.87 (s, 3H) LCMS (ESI) m/z: 411.1 [M+H]⁺

### Example 16. Synthesis of Compound NEM-14A

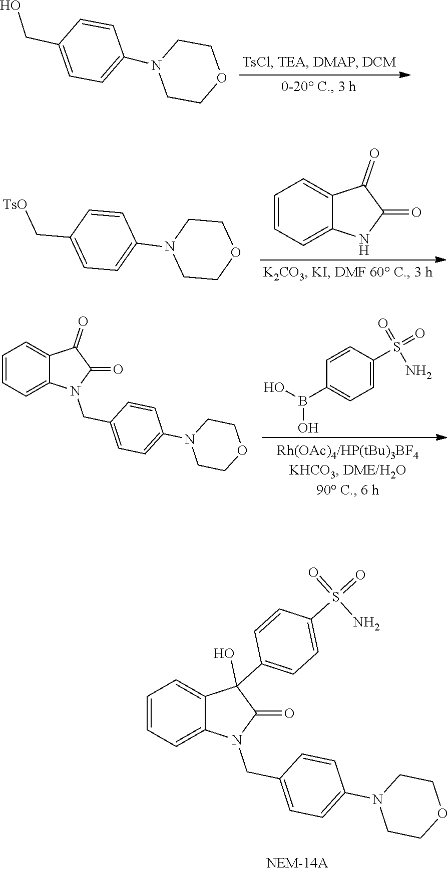

### 1. Synthesis of (4-morpholinophenyl)methyl 4-methylbenzenesulfonate

To a solution of (4-morpholinophenyl)methanol (500 mg, 2.59 mmol, 1 eq) in DCM (5 mL) was added DMAP (63.22 mg, 517.49 umol, 0.2 eq) and TEA (785.46 mg, 7.76 mmol, 1.08 mL, 3 eq). Then 4-methylbenzenesulfonyl chloride (542.62 mg, 2.85 mmol, 1.1 eq) was added to the mixture at 0° C. The mixture was stirred at 20° C. for 1 h. TLC (Dichloromethane. Methanol=10:1) indicated the starting material was remained, and one major new spot with larger polarity was detected. The solvent was removed under pressure to get a solid. The crude product (4-morpholinophenyl)methyl 4-methylbenzenesulfonate (800 mg, crude) was obtained as a yellow solid which was used into the next step without further purification.

### 2. Synthesis of 1-[(4-morpholinophenyl)methyl]indoline-2,3-dione

A mixture of indoline-2,3-dione (350 mg, 2.38 mmol, 1 eq), (4-morpholinophenyl)methyl 4-methylbenzenesulfonate (800 mg, 2.30 mmol, 9.68e-1 eq), KI (157.96 mg, 951.54 umol, 0.4 eq), K₂CO₃ (657.54 mg, 4.76 mmol, 2 eq) in DMF (10 mL) was degassed and purged with N₂ for 3 times, and then the mixture was stirred at 60° C. for 2 h. The reaction mixture was cooled to room temperature and diluted by water (15 mL), extracted with ethyl acetate (20 mL*2). The organic layer was washed with brine (5 mL), dried over Na₂SO₄ and concentrated to give crude product. The crude product was purified by flash column (ISCO 40 g silica, 0-100% ethyl acetate in petroleum ether, gradient over 20 min). Compound 1-[(4-morpholinophenyl)methyl]indoline-2,3-dione (350 mg, 1.09 mmol, 45.64%) was obtained as a yellow solid. LCMS (ESI) m/z: 323.2 [M+H]⁺

### 3. Synthesis of 4-[3-hydroxy-1-[(4-morpholinophenyl)methyl]-2-oxo-indolin-3-yl]benzenesulfonamide (NEM-14A)

A mixture of 1-[(4-morpholinophenyl)methyl]indoline-2,3-dione (300 mg, 930.64 umol, 1 eq), (4-sulfamoylphenyl)boronic acid (243.19 mg, 1.21 mmol, 1.3 eq), diacetoxyrhodium (2.06 mg, 9.31 umol, 0.01 eq), K₂CO₃ (6.43 mg, 46.53 umol, 0.05 eq) and tritert-butylphosphonium;tetrafluoroborate (8.10 mg, 27.92 umol, 0.03 eq) in DME (3 mL) and H₂O (3 mL) was degassed and purged with N₂ for 3 times, and then the mixture was stirred at 90° C. for 5 hr under N₂ atmosphere. The reaction mixture was cooled to room temperature and diluted by water (15 mL), extracted with ethyl acetate (20 mL*2). The organic layer was washed with brine (5 mL), dried over Na₂SO₄ and concentrated to give crude product. The crude product was purified by preparative HPLC (column: Phenomenex C18 80*30 mm*3 um;mobile phase: [water(TFA)-ACN];B %: 25%-55%, 8 min). Compound 4-[3-hydroxy-1-[(4-morpholinophenyl)methyl]-2-oxo-indolin-3-yl]benzenesulfonamide (23.01 mg, 47.02 umol, 5.05%, 98% purity) was obtained as a pale yellow solid. ¹H NMR (400 MHz, ACETONITRILE-d3) δ 7.84-7.82 (d, J=8.4, 2H), 7.52-7.50 (d, J=8.4, 2H), 7.34-7.29 (m, 3H), 7.22-7.18 (m, 1H), 7.12-7.05 (m, 3H), 6.96 (d, J=8.0 Hz, 1H), 5.66 (s, 2H), 4.86 (d, J=2.6 Hz, 2H), 3.86-3.80 (m, 4H), 3.25-3.19 (m, 4H) LCMS (ESI) m/z: 480.1 [M+H]⁺.

### Example 17. Synthesis of Compound NEM-15A

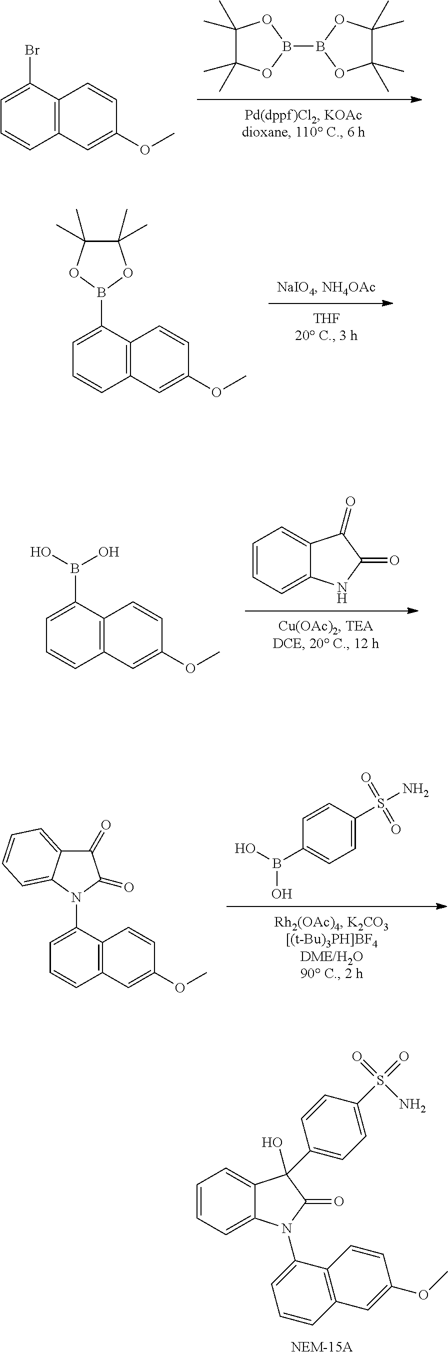

### 1. Synthesis of 2-(6-methoxy-1-naphthyl)-4,4,5,5-tetramethyl-1,3,2-dioxaborolane

A mixture of 1-bromo-6-methoxy-naphthalene (800 mg, 3.37 mmol, 1 eq), 4,4,5,5-tetramethyl-2-(4,4,5,5-tetramethyl-1,3,2-dioxaborolan-2-yl)-1,3,2-dioxaborolane (1.11 g, 4.39 mmol, 1.3 eq), Pd(dppf)Cl₂·CH₂Cl₂ (551.10 mg, 674.84 umol, 0.2 eq), KOAc (993.46 mg, 10.12 mmol, 3 eq) in dioxane (10 mL) was degassed and purged with N₂ for 3 times, and then the mixture was stirred at 120° C. for 3 h under N₂ atmosphere. After cooling to room temperature, the reaction mixture was filtered through a pad of Celite and the celite was rinsed with ethyl acetate (20 mL*3). The filtrate was diluted with water (20 mL), extracted with ethyl acetate (20 mL*3). The combined organics were washed with brine (10 mL), dried over Na₂SO₄, filtered and concentrated under reduced pressure to give a residue. The crude product was purified by flash chromatography on silica gel (Petroleum ether/EtOAc=50:1-2:1) to give 2-(6-methoxy-1-naphthyl)-4,4,5,5-tetramethyl-1,3,2-dioxaborolane (780 mg, 2.74 mmol, 81.35%) as a yellow solid. LCMS (ESI) m/z: 285.3 [M+H]⁺.

### 2. Synthesis of (6-methoxy-1-naphthyl)boronic acid

To a solution of 2-(6-methoxy-1-naphthyl)-4,4,5,5-tetramethyl-1,3,2-dioxaborolane (750 mg, 2.64 mmol, 1 eq) in THE (5 mL) was added NaIO₄ (2.26 g, 10.56 mmol, 585.02 uL, 4 eq), NH₄OAc (813.80 mg, 10.56 mmol, 4 eq) and H₂O (5 mL). The mixture was stirred at 20° C. for 12 h. The reaction mixture was quenched by addition Na₂SO₃ (10 mL) at 0° C., and then diluted with H₂O (10 mL) and extracted with EtOAc 50 mL (25 mL*2). The combined organic layers were washed with brine (20 mL), dried over Na₂SO₄, filtered and concentrated under reduced pressure to give a residue. The residue was by flash chromatography on silica gel (Petroleum ether/EtOAc=30:1-1:10) to give a solid. Compound (6-methoxy-1-naphthyl)boronic acid (300 mg, 1.49 mmol, 56.26%) was obtained as a yellow solid. LCMS (ESI) m/z: 203.2 [M+H]⁺

### 3. Synthesis of 1-(6-methoxy-1-naphthyl)indoline-2,3-dione

A mixture of (6-methoxy-1-naphthyl)boronic acid (300 mg, 1.49 mmol, 1 eq), indoline-2,3-dione (284.04 mg, 1.93 mmol, 1.3 eq), Cu(OAc)₂ (269.74 mg, 1.49 mmol, 1 eq), TEA (300.54 mg, 2.97 mmol, 413.40 uL, 2 eq) in DCE (1 mL) was degassed and purged with N₂ for 3 times, and then the mixture was stirred at 20° C. for 3 h under N₂ atmosphere. The reaction mixture was diluted with H₂O (5 mL) and extracted with EtOAc 20 mL (10 mL*2). The combined organic layers were washed with brine (5 mL), dried over Na₂SO₄, filtered and concentrated under reduced pressure to give a residue. The crude product was purified by flash chromatography on silica gel (Petroleum ether/EtOAc=30:1-1:1) to give 1-(6-methoxy-1-naphthyl)indoline-2,3-dione (150 mg, 494.54 umol, 33.30%) as a yellow solid. LCMS (ESI) m/z: 304.2 [M+H]⁺

### 4. Synthesis of 4-[3-hydroxy-1-(6-methoxy-1-naphthyl)-2-oxo-indolin-3-yl]benzenesulfonamide (NEM-15A)

A mixture of 1-(6-methoxy-1-naphthyl)indoline-2,3-dione (100 mg, 329.69 umol, 1 eq), (4-sulfamoylphenyl)boronic acid (132.54 mg, 659.39 umol, 2 eq), K₂CO₃ (4.56 mg, 32.97 umol, 0.1 eq), diacetoxyrhodium (7.29 mg, 32.97 umol, 0.1 eq) and tritert-butylphosphonium;tetrafluoroborate (9.57 mg, 32.97 umol, 0.1 eq) in DME (1 mL) and H₂O (1 mL) was degassed and purged with N₂ for 3 times, and then the mixture was stirred at 90° C. for 3 h under N₂ atmosphere. The reaction mixture was cooled to room temperature and diluted by water (5 mL), extracted with ethyl acetate (10 mL*2). The organic layer was washed with brine (5 mL), dried over Na₂SO₄ and concentrated to give crude product. The crude product was purified by preparative HPLC (column: C18-1 150*30 mm*5 um;mobile phase: [water(TFA)-ACN];B %: 15%-60%, 8 min) to give 4-[3-hydroxy-1-(6-methoxy-1-naphthyl)-2-oxo-indolin-3-yl]benzenesulfonamide (6.05 mg, 12.98 umol, 3.94%) as a pale yellow solid.

¹H NMR (400 MHz, METHANOL-d4) δ 8.01-7.91 (m, 3H), 7.79-7.69 (m, 2.4H), 7.67-7.58 (m, 1H), 7.45-7.36 (m, 3H), 7.35-7.08 (m, 3.6H), 6.45 (dd, J=7.8, 14.1 Hz, 1H), 3.94 (d, J=8.5 Hz, 3H) LCMS (ESI) m/z: 461.0 [M+H]⁺

### Example 18. Synthesis of Compound NEM-19A

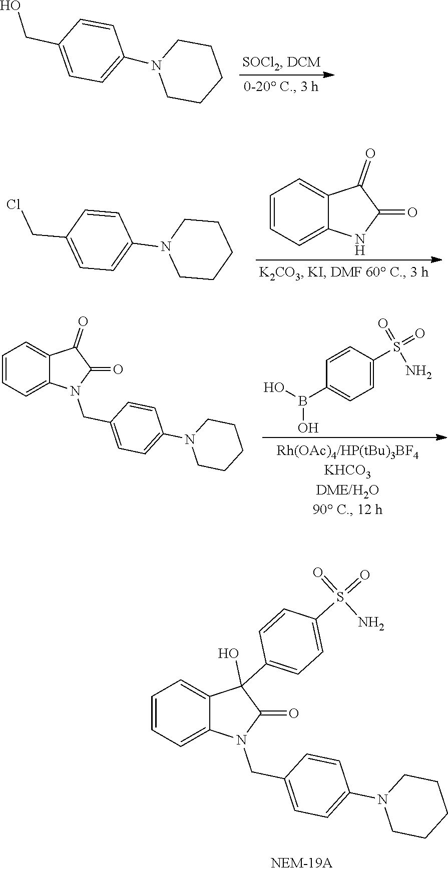

### 1. Synthesis of 1-[4-(chloromethyl)phenyl]piperidine

To a solution of [4-(1-piperidyl)phenyl]methanol (300 mg, 1.57 mmol, 1 eq) in DCM (3 mL) was added SOCl₂ (933.01 mg, 7.84 mmol, 568.91 uL, 5 eq) at 0° C. The mixture was stirred at 20° C. for 2 h. The mixture was concentrated under reduced pressure. The crude product 1-[4-(chloromethyl)phenyl]piperidine (300 mg, crude) was obtained as a white solid which was used into the next step without further purification.

### 2. Synthesis of 1-[[4-(1-piperidyl)phenyl]methyl]indoline-2,3-dione

To a solution of 1-[4-(chloromethyl)phenyl]piperidine (250 mg, 1.19 mmol, 1 eq) in DMF (4 mL) was added K₂CO₃ (329.52 mg, 2.38 mmol, 2 eq), KI (197.89 mg, 1.19 mmol, 1 eq) and indoline-2,3-dione (175.39 mg, 1.19 mmol, 1 eq). The mixture was stirred at 60° C. for 3 h. The reaction mixture was partitioned between EtOAc (50 mL) and brine (30 mL). The organic phase was separated, dried over Na₂SO₄, filtered and concentrated under reduced pressure to give a residue. The residue was purified by flash silica gel chromatography (ISCO®; 10 g SepaFlash® Silica Flash Column, Eluent of 0-100% Ethylacetate/Petroleum ethergradient @50 mL/min). Compound 1-[[4-(1-piperidyl)phenyl]methyl]indoline-2,3-dione (140 mg, crude) was obtained as a yellow solid. LCMS (ESI) m/z: 321.2 [M+H]⁺

### 3. Synthesis of 4-[3-hydroxy-2-oxo-1-[[4-(1-piperidyl)phenyl]methyl]indolin-3-yl]benzenesulfonamide (NEM-19A)

A mixture of 1-[[4-(1-piperidyl)phenyl]methyl]indoline-2,3-dione (100 mg, 312.13 umol, 1 eq), (4-sulfamoylphenyl)boronic acid (94.11 mg, 468.19 umol, 1.5 eq), tritert-butylphosphonium;tetrafluoroborate (3.62 mg, 12.49 umol, 0.04 eq), diacetoxyrhodium (1.38 mg, 6.24 umol, 0.02 eq) and K₂CO₃ (43.14 mg, 312.13 umol, 1 eq) in DME (3 mL) and H₂O (3 mL) was degassed and purged with N₂ for 3 times, and then the mixture was stirred at 90° C. for 3 h under N₂ atmosphere. The reaction mixture was partitioned between EtOAc (30 mL) and H₂O (10 mL). The organic phase was separated, washed with brine (5 mL), dried over Na₂SO₄, filtered and concentrated under reduced pressure to give a residue. The residue was purified by prep-HPLC (column: Waters Xbridge BEH C18 100*30 mm*10 um;mobile phase: [water(NH4HCO3)-ACN];B %: 30%-60%, 8 min). Compound 4-[3-hydroxy-2-oxo-1-[[4-(1-piperidyl)phenyl]methyl]indolin-3-yl]benzenesulfonamide (11.5 mg, 23.61 umol, 7.56%, 98.03% purity) was obtained as a white solid.

¹H NMR (400 MHz, ACETONITRILE-d3) δ 7.83 (d, J=8.5 Hz, 2H), 7.50 (d, J=8.5 Hz, 2H), 7.43-7.36 (m, 4H), 7.35-7.30 (m, 1H), 7.21 (d, J=7.4 Hz, 1H), 7.12-7.06 (m, 1H), 6.96 (d, J=8.0 Hz, 1H), 5.68 (s, 2H), 4.91 (d, J=2.8 Hz, 2H), 3.39-3.32 (m, 4H), 1.87 (td, J=5.7, 10.9 Hz, 4H), 1.71-1.63 (m, 2H)

### Example 19. Synthesis of Compound NEM-23A

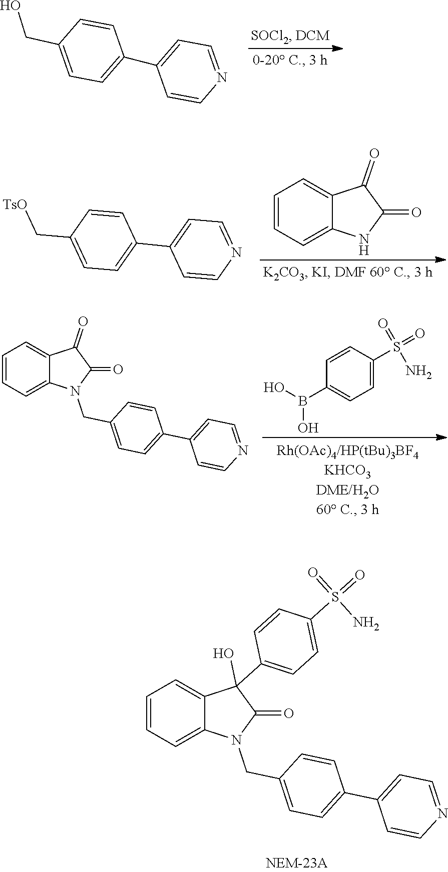

### 1. Synthesis of 4-[4-(chloromethyl)phenyl]pyridine

To a solution of [4-(4-pyridyl)phenyl]methanol (300 mg, 1.62 mmol, 1 eq) in DCM (5 mL) was added SOCl₂ (963.47 mg, 8.10 mmol, 587.48 uL, 5 eq) at 0° C. The mixture was stirred at 20° C. for 2 h. The mixture was concentrated under reduced pressure to give crude product. The crude product 4-[4-(chloromethyl)phenyl]pyridine (300 mg, crude) as a yellow solid which was used into the next step without further purification. LCMS (ESI) m/z: 204.3 [M+H]⁺

### 2. Synthesis of 1-[[4-(4-pyridyl)phenyl]methyl]indoline-2,3-dione

To a solution of 4-[4-(chloromethyl)phenyl]pyridine (150 mg, 736.50 umol, 1 eq), indoline-2,3-dione (108.36 mg, 736.50 umol, 1 eq) in DMF (4 mL) was added K₂CO₃ (203.58 mg, 1.47 mmol, 2 eq) and KI (12.23 mg, 73.65 umol, 0.1 eq). The mixture was stirred at 60° C. for 3 h. The reaction mixture was partitioned between EtOAc (20 mL) and brine (5 mL). The organic phase was separated, dried over Na₂SO₄, filtered and concentrated under reduced pressure to give a residue. The residue was purified by flash silica gel chromatography (ISCO®; 10 g SepaFlash® Silica Flash Column, Eluent of 0-100% Ethylacetate/Petroleum ethergradient @50 mL/min). Compound 1-[[4-(4-pyridyl)phenyl]methyl]indoline-2,3-dione (140 mg, 445.38 umol, 60.47%) was obtained as a yellow solid. LCMS (ESI) m/z: 315.2 [M+H]⁺

### 3. Synthesis of 4-[3-hydroxy-2-oxo-1-[[4-(4-pyridyl)phenyl]methyl]indolin-3-yl]benzenesulfonamide (NEM-23A)

A mixture of 1-[[4-(4-pyridyl)phenyl]methyl]indoline-2,3-dione (100 mg, 318.13 umol, 1 eq), (4-sulfamoylphenyl)boronic acid (95.92 mg, 477.20 umol, 1.5 eq), tritert-butylphosphonium;tetrafluoroborate (3.69 mg, 12.73 umol, 0.04 eq), diacetoxyrhodium (1.41 mg, 6.36 umol, 0.02 eq) and K₂CO₃ (4.40 mg, 31.81 umol, 0.1 eq) in DME (1 mL) and H₂O (1 mL) was degassed and purged with N₂ for 3 times, and then the mixture was stirred at 90° C. for 3 h under N₂ atmosphere. The reaction mixture was partitioned between EtOAc (20 mL) and H₂O (5 mL). The organic phase was separated, washed with brine (5 mL), dried over Na₂SO₄, filtered and concentrated under reduced pressure to give a residue. The residue was purified by prep-HPLC (column: Waters Xbridge BEH C18 100*30 mm*10 um;mobile phase: [water(NH4HCO3)-ACN];B %: 18%-48%, 8 min). Compound 4-[3-hydroxy-2-oxo-1-[[4-(4-pyridyl)phenyl]methyl]indolin-3-yl]benzenesulfonamide (10.5 mg, 22.27 umol, 7.00%, 100% purity) was obtained as a yellow solid.

¹H NMR (400 MHz, METHANOL-d4) δ 8.66 (br d, J=6.1 Hz, 2H), 7.94 (br d, J=6.3 Hz, 2H), 7.85 (br dd, J=8.3, 16.3 Hz, 4H), 7.55 (br t, J=9.2 Hz, 4H), 7.33 (br t, J=7.7 Hz, 1H), 7.24 (br d, J=7.4 Hz, 1H), 7.15-7.09 (m, 1H), 7.02 (d, J=7.9 Hz, 1H), 5.14-5.02 (m, 2H).

### Example 20. Synthesis of Compound NEM-26A

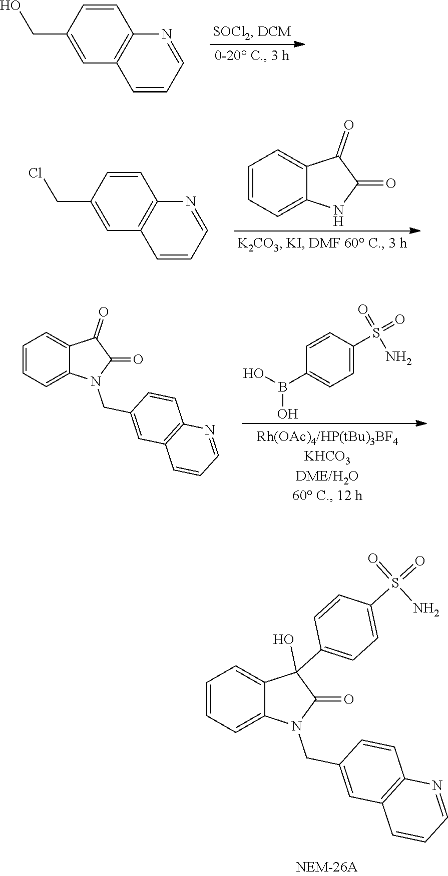 [SMILES: OCc1ccc2ncccc2c1 ; ClCc1ccc2ncccc2c1 ; O=C1Nc2ccccc2C1=O ; O=C1C(=O)N(Cc2ccc3ncccc3c2)c2ccccc21 ; NS(=O)(=O)c1ccc(B(O)O)cc1 ; NS(=O)(=O)c1ccc(C2(O)C(=O)N(Cc3ccc4ncccc4c3)c3ccccc32)cc1]

### 1. Synthesis of 6-(chloromethyl)quinoline

To a solution of 6-quinolylmethanol (500 mg, 3.14 mmol, 1 eq) in CH₃CN (12 mL) was added SOCl₂ (3.28 g, 27.57 mmol, 2 mL, 8.78 eq) at 0° C. The mixture was stirred at 20° C. for 2 h. The mixture was concentrated under reduced pressure. The crude product 6-(chloromethyl)quinoline (300 mg, crude) was obtained as a light yellow solid which was used into the next step without further purification. LCMS (ESI) m/z: 178.3 [M+H]⁺.

### 2. Synthesis of 1-(6-quinolylmethyl)indoline-2,3-dione

To a solution of 6-(chloromethyl)quinoline (300 mg, 1.69 mmol, 1 eq) in DMF (3 mL) was added indoline-2,3-dione (248.49 mg, 1.69 mmol, 1 eq), K₂CO₃ (466.83 mg, 3.38 mmol, 2 eq) and KI (28.04 mg, 168.89 umol, 0.1 eq). The mixture was stirred at 60° C. for 3 hr. The reaction mixture was added H₂O (5 mL), extracted with EtOAc (10 mL*3). The organic phase was dried over Na₂SO₄, filtered and concentrated under reduced pressure to give a residue. The residue was purified by flash silica gel chromatography (ISCO®; 10 g SepaFlash® Silica Flash Column, Eluent of 0-100% Ethylacetate/Petroleum ethergradient @50 mL/min). Compound 1-(6-quinolylmethyl)indoline-2,3-dione (150 mg, crude) was obtained as a yellow solid. LCMS (ESI) m/z: 289.3 [M+H]⁺

### 3. Synthesis of 4-[3-hydroxy-2-oxo-1-(6-quinolylmethyl)indolin-3-yl]benzenesulfonamide (NEM-26A)

A mixture of 1-(6-quinolylmethyl)indoline-2,3-dione (140 mg, 485.61 umol, 1 eq), (4-sulfamoylphenyl)boronic acid (146.42 mg, 728.41 umol, 1.5 eq), tritert-butylphosphonium;tetrafluoroborate (5.64 mg, 19.42 umol, 0.04 eq), diacetoxyrhodium (2.15 mg, 9.71 umol, 0.02 eq) and K₂CO₃ (67.11 mg, 485.61 umol, 1 eq) in DME (3 mL) and H₂O (3 mL) was degassed and purged with N₂ for 3 times, and then the mixture was stirred at 90° C. for 3 h under N₂ atmosphere. The reaction mixture was added H₂O (5 mL), extracted with EtOAc (10 mL*3). The organic phase was dried over Na₂SO₄, filtered and concentrated under reduced pressure to give a residue. The residue was purified by prep-HPLC (column: Waters Xbridge BEH C18 100*30 mm*10 um;mobile phase: [water(NH4HCO3)-ACN];B %: 18%-48%, 8 min). Compound 4-[3-hydroxy-2-oxo-1-(6-quinolylmethyl)indolin-3-yl]benzenesulfonamide (10.5 mg, 23.05 umol, 4.75%, 97.78% purity) was obtained as a white solid.

¹H NMR (400 MHz, METHANOL-d4) δ 8.83 (dd, J=1.5, 4.3 Hz, 1H), 8.31 (d, J=8.0 Hz, 1H), 8.01 (d, J=8.8 Hz, 1H), 7.93 (s, 1H), 7.87 (d, J=8.5 Hz, 2H), 7.77 (dd, J=1.8, 8.8 Hz, 1H), 7.59-7.51 (m, 3H), 7.33-7.27 (m, 1H), 7.24 (d, J=7.0 Hz, 1H), 7.14-7.08 (m, 1H), 7.03 (d, J=7.9 Hz, 1H), 5.25-5.13 (m, 2H)

### Example 21. Synthesis of Compound NEM-20A

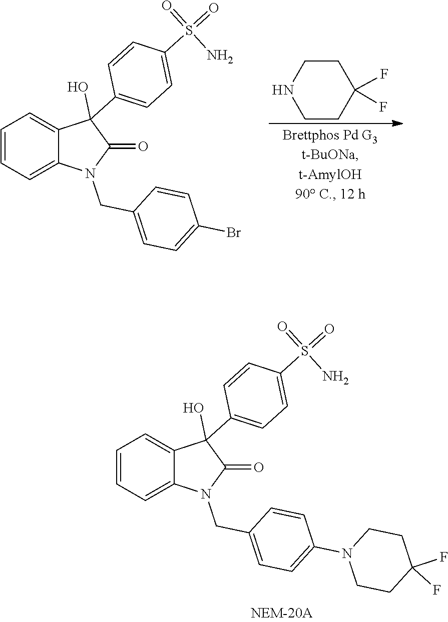 [SMILES: NS(=O)(=O)c1ccc(C2(O)C(=O)N(Cc3ccc(Br)cc3)c3ccccc32)cc1 ; FC1(F)CCNCC1 ; NS(=O)(=O)c1ccc(C2(O)C(=O)N(Cc3ccc(N4CCC(F)(F)CC4)cc3)c3ccccc32)cc1]

### 1. Synthesis of 4-[1-[[4-(4,4-difluoro-1-piperidyl)phenyl]methyl]-3-hydroxy-2-oxo-indolin-3-yl]benzenesulfonamide (NEM-20A)

To a mixture of 4-[1-[(4-bromophenyl)methyl]-3-hydroxy-2-oxo-indolin-3-yl]benzenesulfonamide (130 mg, 274.64 umol, 1 eq), 4,4-difluoropiperidine (49.90 mg, 411.97 umol, 1.5 eq), BrettPhos Pd G₃ (49.79 mg, 54.93 umol, 0.2 eq), sodium;2-methylpropan-2-olate (2 M, 274.64 uL, 2 eq) in t-AmylOH (2 mL) was degassed and purged with N₂ for 3 times, and then the mixture was stirred at 90° C. for 12 h under N₂ atmosphere. The reaction mixture was concentrated under reduced pressure to remove solvent. The residue was purified by prep-HPLC (column: Phenomenex Luna 80*30 mm*3 um;mobile phase: [water(HCl)-ACN];B %: 5%-35%, 8 min). Compound 4-[1-[[4-(4,4-difluoro-1-piperidyl)phenyl]methyl]-3-hydroxy-2-oxo-indolin-3-yl]benzenesulfonamide (21.2 mg, 41.28 umol, 15.03% yield) was obtained as a white solid.

¹H NMR (400 MHz, Chloroform-d) δ 7.87-7.82 (m, 2H), 7.53-7.47 (m, 2H), 7.33-7.29 (m, 1H), 7.26-7.19 (m, 3H), 7.10-7.05 (m, 1H), 6.94 (br d, J=6.1 Hz, 2H), 6.89 (d, J=7.9 Hz, 1H), 5.04-4.94 (m, 3H), 4.78 (d, J=15.4 Hz, 1H), 3.85 (br s, 1H), 3.36 (br t, J=5.6 Hz, 4H), 2.12 (br s, 4H) MS (M+H)⁺=514.1.

### Example 22. Synthesis of Compounds NEM-30A and NEM-21A

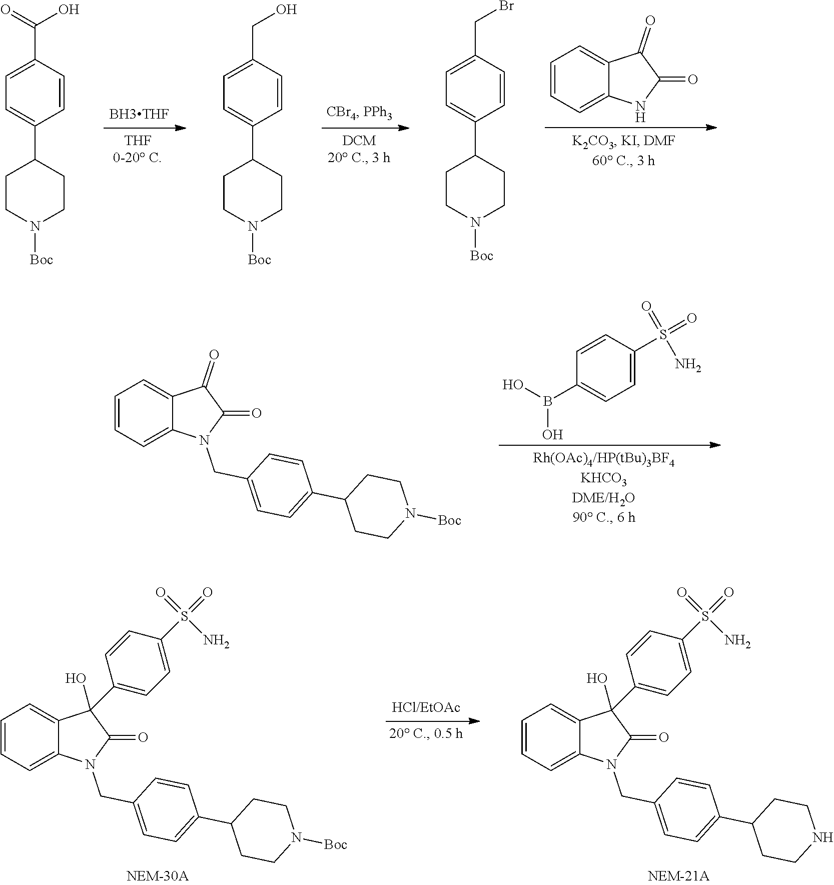

### 1. Synthesis of tert-butyl 4-[4-(hydroxymethyl)phenyl]piperidine-1-carboxylate

To a solution of 4-(1-tert-butoxycarbonyl-4-piperidyl)benzoic acid (1.3 g, 4.26 mmol, 1 eq) in THE (15 mL) was added BH₃·THF (1 M, 8.51 mL, 2 eq) dropwise at 0° C. The mixture was stirred at 20° C. for 12 h. The mixture was quenched with 5% aq. NH₄C₁ (10 mL), extracted with ethyl acetate (30 mL). The organic layer was washed with aq NaHCO₃ (25 mL), dried with Na₂SO₄ and concentrated. Compound tert-butyl 4-[4-(hydroxymethyl)phenyl]piperidine-1-carboxylate (1 g, crude) was obtained as a white solid which was used into next step without further purification.

### 2. Synthesis of tert-butyl 4-[4-(bromomethyl)phenyl]piperidine-1-carboxylate

To a solution of tert-butyl 4-[4-(hydroxymethyl)phenyl]piperidine-1-carboxylate (500 mg, 1.72 mmol, 1 eq) in DCM (10 mL) was added PPh₃ (585.09 mg, 2.23 mmol, 1.3 eq) and CBr₄ (739.77 mg, 2.23 mmol, 1.3 eq). The mixture was stirred at 20° C. for 12 h. The mixture was concentrated under reduced pressure. The crude product tert-butyl 4-[4-(bromomethyl)phenyl]piperidine-1-carboxylate (500 mg, crude) was obtained as a brown oil which was used into the next step without further purification.

### 3. Synthesis of tert-butyl 4-[4-[(2,3-dioxoindolin-1-yl)methyl]phenyl]piperidine-1-carboxylate

To a solution of indoline-2,3-dione (150 mg, 1.02 mmol, 1 eq), tert-butyl 4-[4-(bromomethyl)phenyl]piperidine-1-carboxylate (361.19 mg, 1.02 mmol, 1 eq) in CH₃CN (5 mL) was added K₂CO₃ (281.80 mg, 2.04 mmol, 2 eq) and KI (16.92 mg, 101.95 umol, 0.1 eq). The mixture was stirred at 20° C. for 12 h. The mixture was diluted with H₂O (3 mL), extracted with EtOAc (3×5 mL). The organic layer was washed with brine (3 mL), dried over Na₂SO₄ and concentrated to give crude product. The residue was purified by prep-HPLC (column: Waters Xbridge BEH C18 100*30 mm*10 um;mobile phase: [water(NH4HCO3)-ACN];B %: 45%-75%, 8 min). Compound tert-butyl 4-[4-[(2,3-dioxoindolin-1-yl)methyl]phenyl]piperidine-1-carboxylate (130 mg, 309.16 umol, 30.32% yield) was obtained as a yellow solid.

### 4. Synthesis of tert-butyl 4-[4-[[3-hydroxy-2-oxo-3-(4-sulfamoylphenyl)indolin-1-yl]m ethyl]phenyl]piperidine-1-carboxylate (NEM-30A)

A mixture of tert-butyl 4-[4-[(2,3-dioxoindolin-1-yl)methyl]phenyl]piperidine-1-carboxylate (120 mg, 285.37 umol, 1 eq), (4-sulfamoylphenyl)boronic acid (114.73 mg, 570.75 umol, 2 eq), tritert-butylphosphonium;tetrafluoroborate (2.48 mg, 8.56 umol, 0.03 eq), diacetoxyrhodium (630.65 ug, 2.85 umol, 0.01 eq) and K₂CO₃ (1.97 mg, 14.27 umol, 0.05 eq) in H₂O (2 mL) and DME (2 mL) was degassed and purged with N₂ for 3 times, and then the mixture was stirred at 100° C. for 5 h under N₂ atmosphere. The reaction mixture was concentrated under reduced pressure to remove solvent. The residue was purified by prep-HPLC (column: Waters Xbridge Prep OBD C18 150*40 mm*10 um;mobile phase: [water(NH4HCO3)-ACN];B %: 40%-70%, 8 min). Compound tert-butyl 4-[4-[[3-hydroxy-2-oxo-3-(4-sulfamoylphenyl)indolin-1-yl]methyl]phenyl]piperidine-1-carboxylate (35 mg, 60.59 umol, 21.23% yield) was obtained as a white solid.

¹H NMR (400 MHz, METHANOL-d4) δ 7.85 (d, J=8.5 Hz, 2H), 7.50 (d, J=8.5 Hz, 2H), 7.33-7.28 (m, 3H), 7.24-7.18 (m, 3H), 7.11-7.06 (m, 1H), 6.98 (d, J=7.9 Hz, 1H), 4.99-4.88 (m, 2H), 4.19 (br d, J=13.3 Hz, 2H), 2.85 (br s, 2H), 2.70 (tt, J=3.4, 12.1 Hz, 1H), 1.78 (br s, 2H), 1.63-1.50 (m, 2H), 1.49-1.45 (m, 9H)

### 5. 4-[3-hydroxy-2-oxo-1-[[4-(4-piperidyl)phenyl]methyl]indolin-3-yl]benzenesulfonamide (NEM-21A)

A mixture of tert-butyl 4-[4-[[3-hydroxy-2-oxo-3-(4-sulfamoylphenyl)indolin-1-yl]methyl]phenyl]piperidine-1-carboxylate (28 mg, 48.47 umol, 1 eq) in HCl/EtOAc (4 M, 1.87 mL, 154.05 eq) was stirred at 20° C. for 1 h under N₂ atmosphere. The reaction mixture was concentrated under reduced pressure to remove solvent. The residue was purified by prep-HPLC (column: Phenomenex Luna 80*30 mm*3 um;mobile phase: [water(HCl)-ACN];B %: 5%-35%, 8 min). Compound 4-[3-hydroxy-2-oxo-1-[[4-(4-piperidyl)phenyl]methyl]indolin-3-yl]benzenesulfonamide (20 mg, 41.88 umol, 86.40% yield) was obtained as a pale yellow solid.

¹H NMR (400 MHz, METHANOL-d4) δ 7.86-7.84 (d, J=8.4 Hz, 2H), 7.50 (d, J=8.4 Hz, 2H), 7.38-7.35 (m, 2H), 7.33-7.23 (m, 3H), 7.20-7.16 (m, 1H), 7.10-7.05 (m, 1H), 7.01-6.95 (m, 1H), 4.99-4.88 (m, 2H), 3.50-3.47 (m, 2H), 3.16-3.09 (m, 2H), 2.91-2.85 (m, 1H), 2.07-2.03 (m, 2H), 1.90-1.84 (m, 2H) MS (M+H)+=478.0.

### Example 23. Synthesis of Compounds NEM-32A and NEM-22A

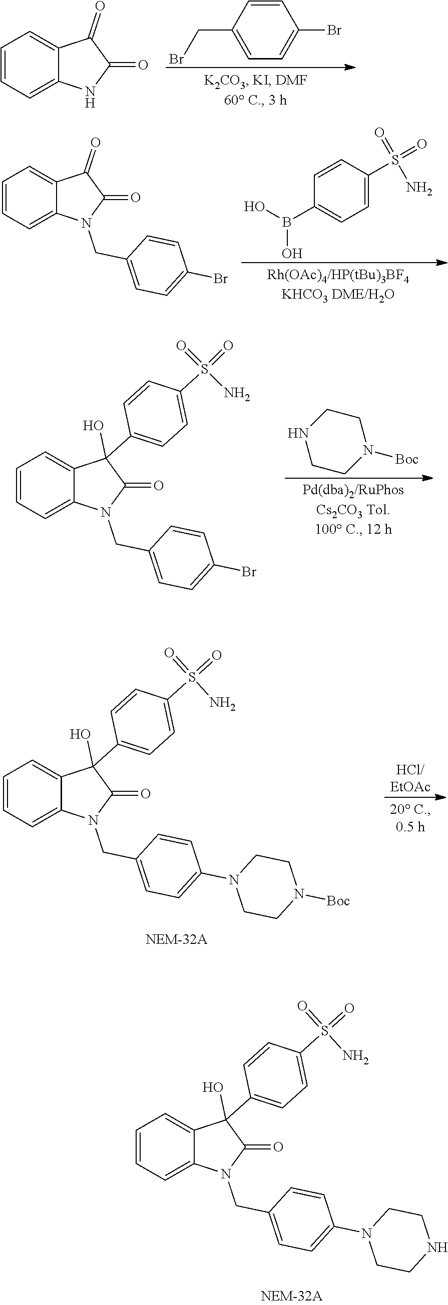

### 1. Synthesis of 1-[(4-bromophenyl)methyl]indoline-2,3-dione

To a solution of indoline-2,3-dione (1 g, 6.80 mmol, 1 eq), 1-bromo-4-(bromomethyl)benzene (1.70 g, 6.80 mmol, 1 eq) in CH₃CN (10 mL) was added K₂CO₃ (1.88 g, 13.60 mmol, 2 eq) and KI (112.83 mg, 680.00 umol, 0.1 eq). The mixture was stirred at 20° C. for 12 h. The mixture was diluted with water (20 mL) and extracted with ethyl acetate (3×20 mL). The combined organic layers were dried over sodium sulphate and concentrated in vacuo to give a residue. The residue was purified by column chromatography (silica gel, 100-200 mesh, 0-50% ethyl acetate in petroleum ether). Compound 1-[(4-bromophenyl)methyl]indoline-2,3-dione (1.5 g, 4.74 mmol, 69.77% yield) was obtained as a red solid.

### 2. Synthesis of 4-[1-[(4-bromophenyl)methyl]-3-hydroxy-2-oxo-indolin-3-yl]benzenesulfonamide

A mixture of [4-(tert-butylsulfamoyl)phenyl]boronic acid (400 mg, 1.27 mmol, 1 eq), (4-sulfamoylphenyl)boronic acid (382.92 mg, 1.91 mmol, 1.5 eq), diacetoxyrhodium (5.61 mg, 25.40 umol, 0.02 eq), tritert-butylphosphonium;tetrafluoroborate (14.74 mg, 50.80 umol, 0.04 eq) and K₂CO₃ (17.55 mg, 127.00 umol, 0.1 eq) in DME (3 mL) and H₂O (3 mL) was degassed and purged with N₂ for 3 times and then the mixture was stirred at 90° C. for 3 h under N₂ atmosphere. The reaction mixture was partitioned between ethyl acetate (50 mL) and H₂O (30 mL). The organic phase was separated, washed with brine (10 mL), dried over Na₂SO₄, filtered and concentrated under reduced pressure to give a residue. The residue was purified by column chromatography (silica gel, 100-200 mesh, 0-100% ethyl acetate in petroleum ether). Compound 4-[1-[(4-bromophenyl)methyl]-3-hydroxy-2-oxo-indolin-3-yl]benzenesulfonamide (300 mg, 633.80 umol, 49.91% yield) was obtained as a yellow solid.

### 3. Synthesis of tert-butyl 4-[4-[[3-hydroxy-2-oxo-3-(4-sulfamoylphenyl)indolin-1-yl]m ethyl]phenyl]piperazine-1-carboxylate (NEM-32A)

A mixture of 4-[1-[(4-bromophenyl)methyl]-3-hydroxy-2-oxo-indolin-3-yl]benzenesulfonamide 150 mg, 316.90 umol, 1 eq), tert-butyl piperazine-1-carboxylate (118.04 mg, 633.80 umol, 2 eq), BrettPhos Pd G₃ (57.45 mg, 63.38 umol, 0.2 eq), sodium;2-methylpropan-2-olate (2 M, 475.35 uL, 3 eq) in t-AmylOH (5 mL) was degassed and purged with N₂ for 3 times. Then the mixture was stirred at 90° C. for 12 h under N₂ atmosphere. The reaction mixture was concentrated under reduced pressure to remove solvent. The residue was purified by prep-HPLC (column: Waters Xbridge Prep OBD C18 150*40 mm*10 um;mobile phase: [water(NH4HCO3)-ACN];B %: 40%-70%, 8 min). Compound tert-butyl 4-[4-[[3-hydroxy-2-oxo-3-(4-sulfamoylphenyl)indolin-1-yl]methyl]phenyl]piperazine-1-carboxylate (30 mg, 47.36 umol, 14.94% yield) was obtained as a pale yellow solid.

H NMR (400 MHz, Acetonitrile-d3) δ 7.87-7.78 (m, 2H), 7.55-7.48 (m, 2H), 7.30 (dt, J=1.3, 7.8 Hz, 1H), 7.24 (d, J=8.6 Hz, 2H), 7.20-7.16 (m, 1H), 7.08-7.03 (m, 1H), 6.96 (d, J=7.9 Hz, 1H), 6.93-6.89 (m, 2H), 5.74-5.56 (m, 2H), 4.88-4.77 (m, 2H), 3.53-3.46 (m, 4H), 3.10-3.04 (m, 4H), 1.43 (s, 9H).

### 4. Synthesis of 4-[3-hydroxy-2-oxo-1-[(4-piperazin-1-ylphenyl)methyl]indolin-3-yl]benzenesulfonamide (NEM-22A)

A mixture of tert-butyl 4-[4-[[3-hydroxy-2-oxo-3-(4-sulfamoylphenyl)indolin-1-yl]methyl]phenyl]piperazine-1-carboxylate (20 mg, 34.56 umol, 1 eq) in HCl/EtOAc (4 M, 2.00 mL, 231.47 eq) was stirred at 20° C. for 1 hr under N₂ atmosphere. The reaction mixture was concentrated under reduced pressure to remove solvent. The residue was purified by prep-HPLC (column: Phenomenex Luna 80*30 mm*3 um;mobile phase: [water(HCl)-ACN];B %: 1%-30%, 8 min). Compound 4-[3-hydroxy-2-oxo-1-[(4-piperazin-1-ylphenyl)methyl]indolin-3-yl]benzenesulfonamide (8.1 mg, 16.34 umol, 47.27% yield) was obtained as a pale yellow solid.

¹H NMR (400 MHz, METHANOL-d4) δ 7.88-7.82 (m, 2H), 7.48 (d, J=8.5 Hz, 2H), 7.35-7.28 (m, 3H), 7.23-7.18 (m, 1H), 7.12-7.06 (m, 1H), 7.03-6.98 (m, 3H), 4.97-4.92 (m, 1H), 4.82 (s, 1H), 3.42-3.38 (m, 4H), 3.38-3.33 (m, 4H) MS (M+H)+=479.1

### Example 24. Synthesis of Compound NEM-24A

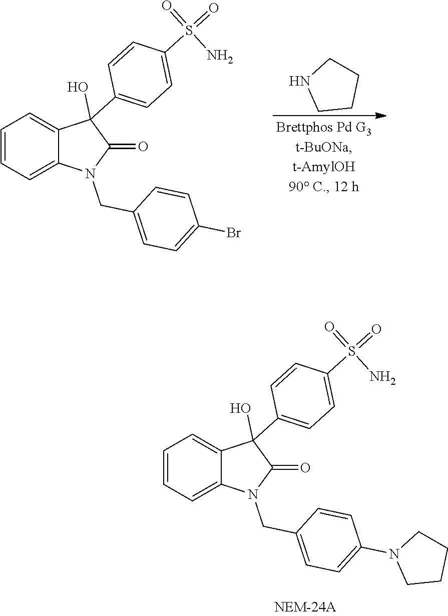 [SMILES: NS(=O)(=O)c1ccc(C2(O)C(=O)N(Cc3ccc(Br)cc3)c3ccccc32)cc1 ; C1CCNC1 ; NS(=O)(=O)c1ccc(C2(O)C(=O)N(Cc3ccc(N4CCCC4)cc3)c3ccccc32)cc1]

### 1. Synthesis of 4-[3-hydroxy-2-oxo-1-[(4-pyrrolidin-1-ylphenyl)methyl]indolin-3-yl]benzenesulfonamide (NEM-24A)

A mixture of 4-[1-[(4-bromophenyl)methyl]-3-hydroxy-2-oxo-indolin-3-yl]benzenesulfonamide (130 mg, 274.64 umol, 1 eq), pyrrolidine (29.30 mg, 411.97 umol, 34.39 uL, 1.5 eq), BrettPhos Pd G₃ (49.79 mg, 54.93 umol, 0.2 eq), sodium;2-methylpropan-2-olate (2 M, 274.64 uL, 2 eq) in t-AmylOH (2 mL) was degassed and purged with N₂ for 3 times, and then the mixture was stirred at 90° C. for 12 h under N₂ atmosphere. The reaction mixture was concentrated under reduced pressure to remove solvent. The residue was purified by prep-HPLC (column: Waters Xbridge BEH C18 100*30 mm*10 um;mobile phase: [water(NH4HCO3)-ACN];B %: 40%-70%, 8 min). Compound 4-[3-hydroxy-2-oxo-1-[(4-pyrrolidin-1-ylphenyl)methyl]indolin-3-yl]benzenesulfonamide (2.3 mg, 4.96 umol, 1.81% yield) was obtained as an off white solid.

¹H NMR (400 MHz, Chloroform-d) δ 7.79 (d, J=8.4 Hz, 2H), 7.44 (d, J=8.4 Hz, 2H), 7.22 (br s, 1H), 7.15-7.10 (m, 3H), 7.01-6.93 (m, 1H), 6.86 (d, J=7.9 Hz, 1H), 6.53-6.43 (m, 2H), 4.92 (d, J=14.9 Hz, 1H), 4.78 (s, 2H), 4.71-4.58 (m, 1H), 3.43 (s, 1H), 3.20 (s, 4H), 1.99-1.89 (m, 4H) MS (M+H)⁺=464.1.

### Example 25. Synthesis of Compound NEM-25A

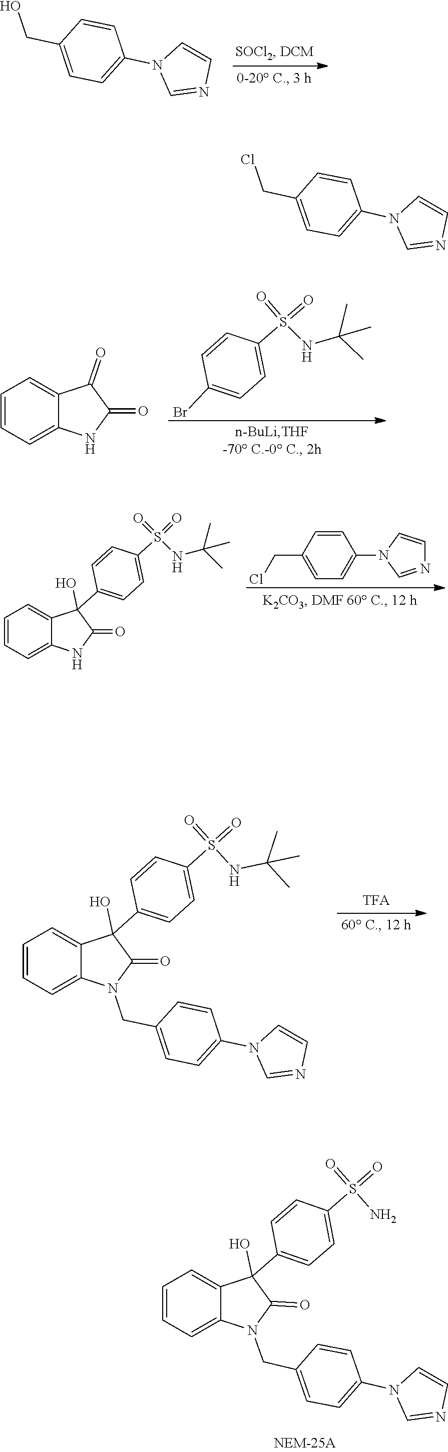

### 1. Synthesis of 1-[4-(chloromethyl)phenyl]imidazole

To a solution of (4-imidazol-1-ylphenyl)methanol (500 mg, 2.87 mmol, 1 eq) in DCM (6 mL) was added SOCl₂ (1.71 g, 14.35 mmol, 1.04 mL, 5 eq) at 0° C. The mixture was stirred at 20° C. for 3 h. The mixture was concentrated under reduced pressure. The mixture was diluted with ethyl acetate (20 mL), washed with sat. NaHCO₃ aqueous at 0° C. until pH=7. The organic layer was washed with brine (5 mL), dried over Na₂SO₄ and concentrated to give crude product. Compound 1-[4-(chloromethyl)phenyl]imidazole (500 mg, crude) was obtained as a white solid which was used directly without further purification.

### 2. Synthesis of N-tert-butyl-4-(3-hydroxy-2-oxo-indolin-3-yl)benzenesulfonamide

To a solution of 4-bromo-N-tert-butyl-benzenesulfonamide (397.19 mg, 1.36 mmol, 2 eq) in THE (4 mL) was added dropwise n-BuLi (2 M, 1.36 mL, 4 eq) at −70° C. After addition, the mixture was stirred at this temperature for 30 min, and then indoline-2,3-dione (100 mg, 679.67 umol, 1 eq) in THE (4 mL) was added dropwise to the above solution at −70° C. The resulting mixture was stirred at −70° C. for 1.5 h. The mixture was quenched with sat. NH₄C₁ (5 mL), extracted with EtOAc (10 mL×3). The organic layer was washed with brine (5 mL), dried over Na₂SO₄, filtered and concentrated under reduced pressure to give a residue. The residue was purified by column chromatography (silica gel, 100-200 mesh, 0-100% ethyl acetate in petroleum ether). Compound N-tert-butyl-4-(3-hydroxy-2-oxo-indolin-3-yl)benzenesulfonamide (100 mg, 277.45 umol, 40.82%) was obtained as a white solid.

¹H NMR (400 MHz, Acetonitrile-d3) δ 8.50 (br s, 1H), 7.84-7.76 (m, 2H), 7.54-7.47 (m, 2H), 7.32 (dt, J=1.3, 7.7 Hz, 1H), 7.18-7.13 (m, 1H), 7.08-6.97 (m, 2H), 5.59 (s, 1H), 4.66 (s, 1H), 1.13 (s, 9H)

### 3. Synthesis of N-tert-butyl-4-[3-hydroxy-1-[(4-imidazol-1-ylphenyl)methyl]-2-oxo-indolin-3-yl]benzenesulfonamide

To a solution of N-tert-butyl-4-(3-hydroxy-2-oxo-indolin-3-yl)benzenesulfonamide (50 mg, 138.72 umol, 1 eq) in DMF (1 mL) was added K₂CO₃ (38.35 mg, 277.45 umol, 2 eq) and 1-[4-(chloromethyl)phenyl]imidazole (32.07 mg, 166.47 umol, 1.2 eq). The mixture was stirred at 60° C. for 12 h. The resultant mixture was filtered and the combined filtrates was purified by prep-HPLC (column: Waters Xbridge BEH C18 100*30 mm*10 um;mobile phase: [water(NH4HCO3)-ACN];B %: 25%-55%, 8 min). Compound N-tert-butyl-4-[3-hydroxy-1-[(4-imidazol-1-ylphenyl)methyl]-2-oxo-indolin-3-yl]benzenesulfonamide (30 mg, 55.17 umol, 39.77% yield, 95% purity) was obtained as a white solid.

1H NMR (400 MHz, METHANOL-d4) δ=8.12 (s, 1H), 7.84 (d, J=8.5 Hz, 2H), 7.58-7.49 (m, 7H), 7.33 (s, 1H), 7.23 (s, 1H), 7.14 (s, 2H), 7.04 (s, 1H), 5.04 (s, 2H), 1.16 (s, 9H)

### 4. Synthesis of 4-[3-hydroxy-1-[(4-imidazol-1-ylphenyl)methyl]-2-oxo-indolin-3-yl]benzenesulfonamide (NEM-25A)

To a solution of N-tert-butyl-4-[3-hydroxy-1-[(4-imidazol-1-ylphenyl)methyl]-2-oxo-indolin-3-yl]benzenesulfonamide (30 mg, 58.07 umol, 1 eq) in DCM (1 mL) was added TFA (2.31 g, 20.26 mmol, 1.5 mL, 348.87 eq). The mixture was stirred at 60° C. for 2 h. The reaction mixture was cooled to 20° C. The solvent was concentrated under reduced pressure to give a residue. The residue was purified by prep-HPLC (column: Phenomenex C18 80*30 mm*3 um;mobile phase: [water(TFA)-ACN];B %: 5%-35%, 8 min). Compound 4-[3-hydroxy-1-[(4-imidazol-1-ylphenyl)methyl]-2-oxo-indolin-3-yl]benzenesulfonamide (13.7 mg, 22.73 umol, 39.14% yield, TFA salt) was obtained as a yellow solid. ¹H NMR (400 MHz, Acetonitrile-d3) δ 8.68 (s, 1H), 7.89-7.82 (m, 2H), 7.66 (br s, 1H), 7.48 (br s, 1H), 7.62-7.46 (m, 7H), 7.34 (dt, J=1.3, 7.8 Hz, 1H), 7.23 (dd, J=0.7, 7.4 Hz, 1H), 7.16-7.07 (m, 1H), 6.99 (d, J=7.9 Hz, 1H), 5.68 (s, 2H), 5.07-4.95 (m, 2H). MS (M+H)⁺=461.1.

### Example 26. Synthesis of Compound NEM-34A

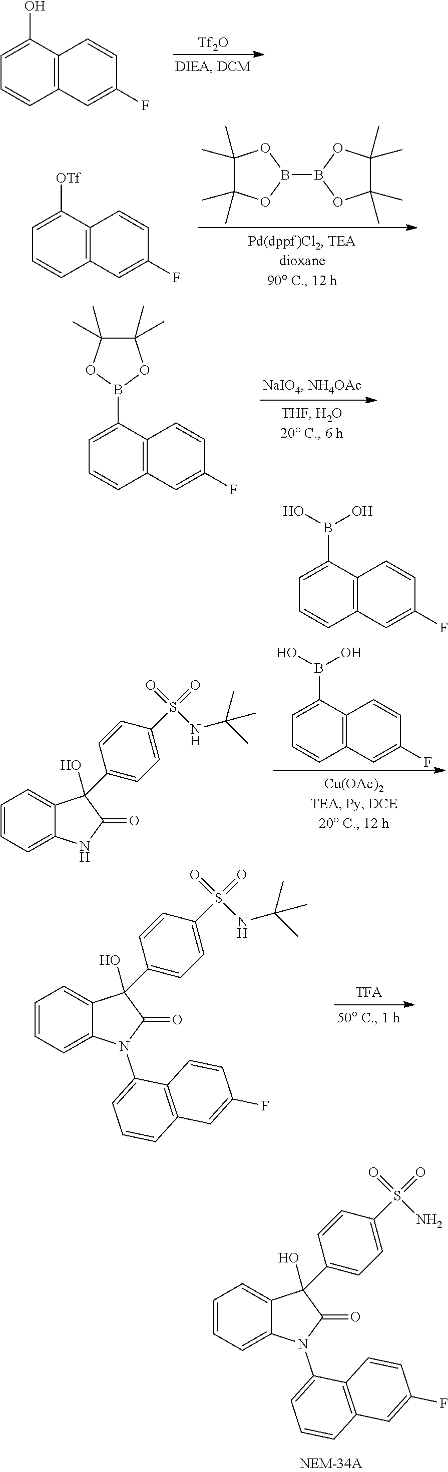

### 1. Synthesis of (6-fluoro-1-naphthyl) trifluoromethanesulfonate

To a solution of 6-fluoronaphthalen-1-ol (500 mg, 3.08 mmol, 1 eq) in DCM (5 mL) was added DIEA (1.59 g, 12.33 mmol, 2.15 mL, 4 eq) and Tf₂O (1.74 g, 6.17 mmol, 1.02 mL, 2 eq). The mixture was stirred at 20° C. for 2 hr. The reaction mixture was diluted by water (30 mL), extracted with DCM (15 mL*3). The combined organics were washed with brine (3 mL), dried over Na₂SO₄ and concentrated under reduced pressure to give a residue. The residue was purified by flash silica gel chromatography (ISCO; 12 g SepaFlash Silica Flash Column, Eluent of 0-15% Ethyl acetate/Petroleum ether gradient @40 mL/min). Compound (6-fluoro-1-naphthyl) trifluoromethanesulfonate (0.85 g, 2.89 mmol, 93.70% yield) was obtained as colorless oil.

### 2. Synthesis of 2-(6-fluoro-1-naphthyl)-4,4,5,5-tetramethyl-1,3,2-dioxaborolane

A mixture of (6-fluoro-1-naphthyl) trifluoromethanesulfonate (674.9 mg, 2.29 mmol, 1 eq), 4,4,5,5-tetramethyl-2-(4,4,5,5-tetramethyl-1,3,2-dioxaborolan-2-yl)-1,3,2-dioxaborolane (2.33 g, 9.18 mmol, 4 eq), cyclopentyl(diphenyl)phosphane;dichloromethane; dichloropalladium;iron (280.99 mg, 344.08 umol, 0.15 eq), TEA (928.45 mg, 9.18 mmol, 1.28 mL, 4 eq) in dioxane (6 mL) was degassed and purged with N₂ for 3 times, and then the mixture was stirred at 90° C. for 12 hr under N₂ atmosphere. The reaction mixture was diluted by water (10 mL), extracted with ethyl acetate (15 mL*3). The combined organics were washed with brine (5 mL), dried over Na₂SO₄, and concentrated under reduced pressure to give a residue. The residue was purified by flash silica gel chromatography (ISCO; 40 g SepaFlash Silica Flash Column, Eluent of 0˜10% Ethyl acetate/Petroleum ether gradient @40 mL/min). Compound 2-(6-fluoro-1-naphthyl)-4,4,5,5-tetramethyl-1,3,2-dioxaborolane (0.5 g, 1.84 mmol, 80.10% yield) was obtained as a light yellow oil. ¹H NMR (400 MHz, CHLOROFORM-d) δ 8.82-8.76 (m, 1H), 8.05-8.03 (m, 1H), 7.90-7.86 (d, J=8.4, 1H), 7.53-7.42 (m, 2H), 7.34-7.28 (m, 1H), 1.43 (s, 12H).

### 3. Synthesis of (6-fluoro-1-naphthyl)boronic acid

To a solution of 2-(6-fluoro-1-naphthyl)-4,4,5,5-tetramethyl-1,3,2-dioxaborolane (470 mg, 1.73 mmol, 1 eq) in THE (9 mL) and H₂O (3 mL) was added NH₄OAc (199.70 mg, 2.59 mmol, 1.5 eq) and NaIO₄ (1.85 g, 8.64 mmol, 478.53 uL, 5 eq). The mixture was stirred at 20° C. for 2 hr. The mixture was acidified by adding hydrochloric acid (1 M, 10 mL) dropwise at 0° C. to pH=6 and the reaction mixture was concentrated. The solid was collected by filtration and was dried under reduced pressure. (6-fluoro-1-naphthyl)boronic acid (350 mg, crude) was obtained as a white solid.

### 4. Synthesis of N-tert-butyl-4-[1-(6-fluoro-1-naphthyl)-3-hydroxy-2-oxo-indolin-3-yl]benzenesulfonamide

A mixture of N-tert-butyl-4-(3-hydroxy-2-oxo-indolin-3-yl)benzenesulfonamide (100 mg, 277.45 umol, 1 eq), (6-fluoro-1-naphthyl)boronic acid (105.42 mg, 554.90 umol, 2 eq), TEA (56.15 mg, 554.90 umol, 77.23 uL, 2 eq), Pyridine (43.89 mg, 554.90 umol, 44.79 uL, 2 eq) and Cu(OAc)₂ (100.79 mg, 554.90 umol, 2 eq) in DCE (2 mL) was degassed and purged with 02 for 3 times, and then the mixture was stirred at 20° C. for 12 hr under 02 atmosphere. 10 mL of water was added to the mixture, the mixture was extracted with dichloromethane (10 mL*3), and the combined extracts were washed with brine (5 mL), dried with anhydrous Na₂SO₄ and concentrated under reduced pressure to give a residue. The residue was purified by flash silica gel chromatography (ISCO; 25 g SepaFlash Silica Flash Column, Eluent of 0-100% Ethyl acetate/Petroleum ether gradient @40 mL/min). Compound N-tert-butyl-4-[1-(6-fluoro-1-naphthyl)-3-hydroxy-2-oxo-indolin-3-yl]benzenesulfonamide (0.07 g, 138.73 umol, 50.00% yield) was obtained as a light yellow solid. MS (M+H)⁺=505.0

### 5. 4-[1-(6-fluoro-1-naphthyl)-3-hydroxy-2-oxo-indolin-3-yl]benzenesulfonamide (NE M-34A)

To a solution of N-tert-butyl-4-[1-(6-fluoro-1-naphthyl)-3-hydroxy-2-oxo-indolin-3-yl]benzenesulfonamide (50 mg, 99.09 umol, 1 eq) in DCM (1 mL) was added TFA (1.93 g, 16.88 mmol, 1.25 mL, 170.37 eq). The mixture was stirred at 50° C. for 1 hr. The mixture was evaporated under reduced pressure. The residue was purified by preparative HPLC (column: Phenomenex Luna C18 75*30 mm*3 um;mobile phase: [water(TFA)-ACN];B %: 35%-65%, 8 min). Compound 4-[1-(6-fluoro-1-naphthyl)-3-hydroxy-2-oxo-indolin-3-yl]benzenesulfonamide (8.5 mg, 15.11 umol, 15.25% yield, TFA salt) was obtained as a yellow solid. ¹H NMR (400 MHz, Acetonitrile-d3) δ 8.08-8.06 (m, 1H), 7.94-7.89 (m, 2H), 7.80-7.65 (m, 4.5H), 7.66-7.56 (m, 1.5H), 7.47-7.24 (m, 3H), 7.23-7.11 (m, 1H), 6.48 (dd, J=7.8, 10.6 Hz, 1H), 5.68 (br d, J=5.6 Hz, 2H). MS (M+H)⁺=449.0.

### Example 27. Synthesis of Compound NEM-35A

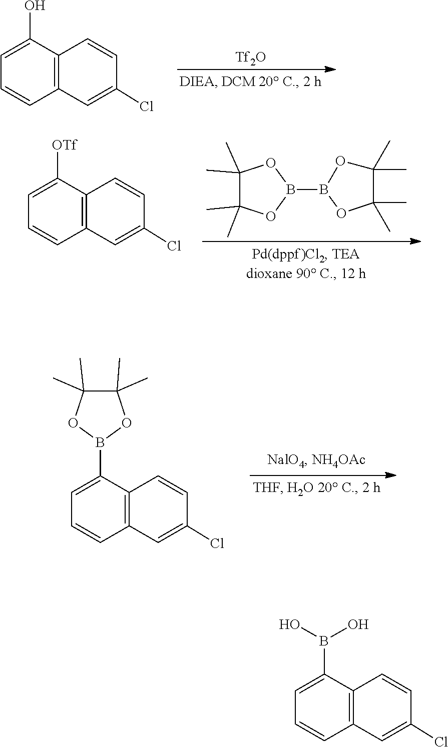

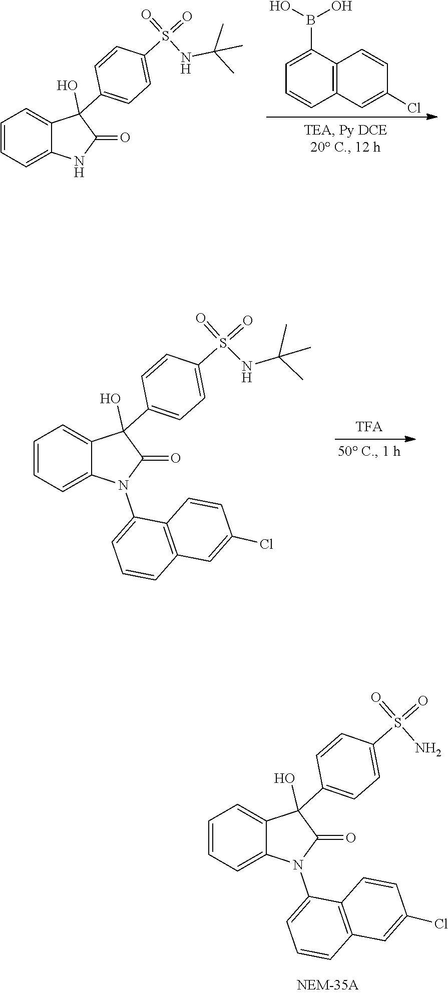 [SMILES: CC(C)(C)NS(=O)(=O)c1ccc(C2(O)C(=O)Nc3ccccc32)cc1 ; OB(O)c1cccc2cc(Cl)ccc12 ; CC(C)(C)NS(=O)(=O)c1ccc(C2(O)C(=O)N(c3cccc4cc(Cl)ccc34)c3ccccc32)cc1 ; NS(=O)(=O)c1ccc(C2(O)C(=O)N(c3cccc4cc(Cl)ccc34)c3ccccc32)cc1]

### 1. Synthesis of (6-chloro-1-naphthyl) trifluoromethanesulfonate

To a solution of 6-chloronaphthalen-1-ol (500 mg, 2.80 mmol, 1 eq) in DCM (6 mL) was added DIEA (1.45 g, 11.20 mmol, 1.95 mL, 4 eq) and Tf₂O (1.58 g, 5.60 mmol, 923.74 uL, 2 eq). The mixture was stirred at 20° C. for 2 hr. 10 mL of water was added to the mixture, the mixture was extracted with dichloromethane (10 mL*3), the combined extracts were washed with brine (10 mL), dried with anhydrous Na₂SO₄ and concentrated under reduced pressure to give a residue. The residue was purified by flash silica gel chromatography (ISCO; 25 g SepaFlash Silica Flash Column, Eluent of 0˜10% Ethyl acetate/Petroleum ether gradient @80 mL/min). Compound (6-chloro-1-naphthyl) trifluoromethanesulfonate (0.83 g, 2.67 mmol, 95.44% yield) was obtained as a light yellow oil. ¹H NMR (400 MHz, CHLOROFORM-d) δ 8.03 (d, J=9.0 Hz, 1H), 7.92 (d, J=1.9 Hz, 1H), 7.80 (d, J=8.1 Hz, 1H), 7.60 (dd, J=2.0, 9.1 Hz, 1H), 7.57-7.50 (m, 1H), 7.50-7.45 (m, 1H)

### 2. Synthesis of 2-(6-chloro-1-naphthyl)-4,4,5,5-tetramethyl-1,3,2-dioxaborolane

A mixture of (6-chloro-1-naphthyl) trifluoromethanesulfonate (830 mg, 2.67 mmol, 1 eq), 4,4,5,5-tetramethyl-2-(4,4,5,5-tetramethyl-1,3,2-dioxaborolan-2-yl)-1,3,2-dioxaborolane (2.71 g, 10.69 mmol, 4 eq), TEA (1.08 g, 10.69 mmol, 1.49 mL, 4 eq), cyclopentyl(diphenyl)phosphane;dichloromethane;dichloropalladium;iron (218.17 mg, 267.16 umol, 0.1 eq) in dioxane (10 mL) was degassed and purged with N₂ for 3 times, and then the mixture was stirred at 90° C. for 12 hr under N₂ atmosphere. The reaction mixture was diluted by water (10 mL), extracted with ethyl acetate (10 mL*3). The combined organics were washed with brine (10 mL), dried over Na₂SO₄ and concentrated under reduced pressure to give a residue. The residue was purified by flash silica gel chromatography (ISCO; 12 g SepaFlash Silica Flash Column, Eluent of 0-1% Ethyl acetate/Petroleum ether gradient @40 mL/min). Compound 2-(6-chloro-1-naphthyl)-4,4,5,5-tetramethyl-1,3,2-dioxaborolane (0.7258 g, 2.52 mmol, 94.14% yield) was obtained as light yellow oil. ¹H NMR (400 MHz, CHLOROFORM-d) δ 8.73 (d, J=9.1 Hz, 1H), 8.08 (dd, J=1.2, 6.8 Hz, 1H), 7.87-7.79 (m, 2H), 7.53-7.44 (m, 2H), 1.43 (s, 12H)

### 3. Synthesis of (6-chloro-1-naphthyl)boronic acid

To a solution of 2-(6-chloro-1-naphthyl)-4,4,5,5-tetramethyl-1,3,2-dioxaborolane (725 mg, 2.51 mmol, 1 eq) in THF (9 mL) and H₂O (3 mL) was added NaIO₄ (2.69 g, 12.56 mmol, 696.07 uL, 5 eq) and NH₄OAc (290.48 mg, 3.77 mmol, 1.5 eq). The mixture was stirred at 20° C. for 2 hr. The mixture was acidified by adding hydrochloric acid (1 M, 10 mL) dropwise at 0° C. to pH=6. The reaction mixture was concentrated and the solid was collected by filtration. (6-chloro-1-naphthyl)boronic acid (300 mg, crude) was obtained as a white solid.

### 4. Synthesis of N-tert-butyl-4-[1-(6-chloro-1-naphthyl)-3-hydroxy-2-oxo-indolin-3-yl]benzenesulfonamide

A mixture of N-tert-butyl-4-(3-hydroxy-2-oxo-indolin-3-yl)benzenesulfonamide (100 mg, 277.45 umol, 1 eq), (6-chloro-1-naphthyl)boronic acid (103.09 mg, 499.41 umol, 1.8 eq), TEA (56.15 mg, 554.90 umol, 77.24 uL, 2 eq), Pyridine (43.89 mg, 554.90 umol, 44.79 uL, 2 eq) and Cu(OAc)₂ (100.79 mg, 554.90 umol, 2 eq) in DCE (1 mL) was degassed and purged with 02 for 3 times, and then the mixture was stirred at 20° C. for 12 hr under 02 atmosphere. 5 mL of water was added to the mixture, the mixture was extracted with DCM (10 mL*3), and the combined extracts were washed with brine (5 mL), dried with anhydrous Na₂SO₄ and concentrated under reduced pressure to give a residue. The residue was purified by preparative HPLC (neutral condition,column: Phenomenex C18 80*40 mm*3 um;mobile phase: [water(NH4HCO3)-ACN];B %: 50%-80%, 8 min). Compound N-tert-butyl-4-[1-(6-chloro-1-naphthyl)-3-hydroxy-2-oxo-indolin-3-yl]benzenesulfonamide (50 mg, 95.96 umol, 34.59% yield) was obtained as a light yellow solid. MS (M+H)⁺=521.1

### 5. 4-[1-(6-chloro-1-naphthyl)-3-hydroxy-2-oxo-indolin-3-yl]benzenesulfonamide (NEM-35A)

To a solution of N-tert-butyl-4-[1-(6-chloro-1-naphthyl)-3-hydroxy-2-oxo-indolin-3-yl]benzenesulfonamide (30 mg, 57.58 umol, 1 eq) in DCM (1 mL) was added TFA (2.31 g, 20.26 mmol, 1.50 mL, 351.86 eq). The mixture was stirred at 50° C. for 1 hr. The mixture was evaporated under reduced pressure. The residue was purified by preparative HPLC (column: Phenomenex Luna C18 75*30 mm*3 um;mobile phase: [water(TFA)-ACN];B %: 35%-65%, 8 min). Compound 4-[1-(6-chloro-1-naphthyl)-3-hydroxy-2-oxo-indolin-3-yl]benzenesulfonamide (12.5 mg, 26.62 umol, 46.23% yield) was obtained as a yellow solid. ¹H NMR (400 MHz, ACETONITRILE-d3) δ 8.11 (dd, J=2.1, 7.8 Hz, 1H), 8.05 (dd, J=5.0, 8.3 Hz, 1H), 7.97-7.87 (m, 2H), 7.78-7.69 (m, 3.5H), 7.64 (dd, J=0.9, 7.3 Hz, 1H), 7.55 (td, J=2.7, 9.0 Hz, 1H), 7.49-7.44 (m, 0.5H), 7.40-7.24 (m, 2H), 7.22-7.12 (m, 1H), 6.48 (dd, J=7.9, 10.1 Hz, 1H), 5.68 (br d, J=5.5 Hz, 2H). MS (M+H)+=464.9

### Example 28. Synthesis of Compound NEM-36A

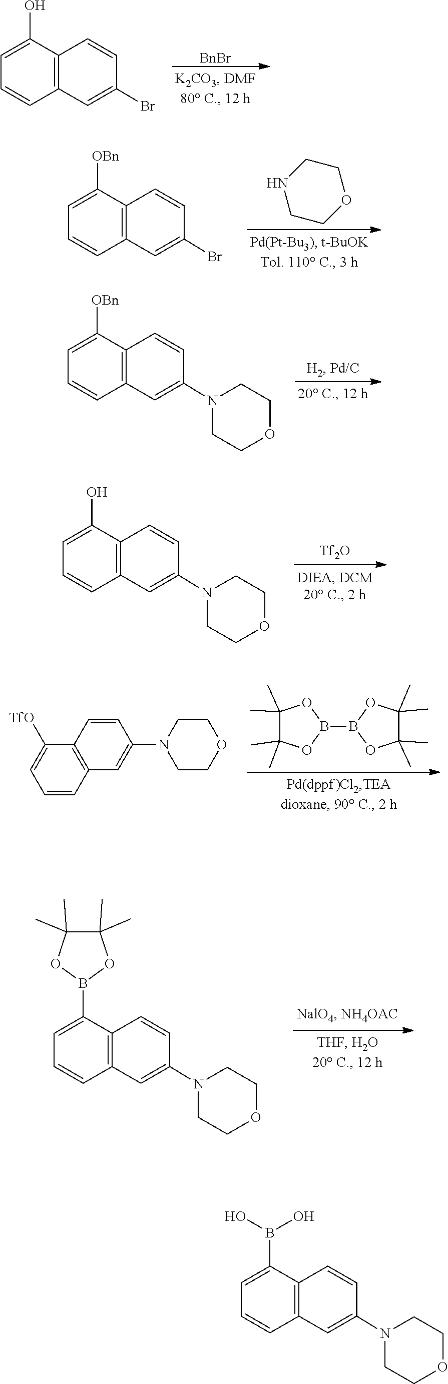

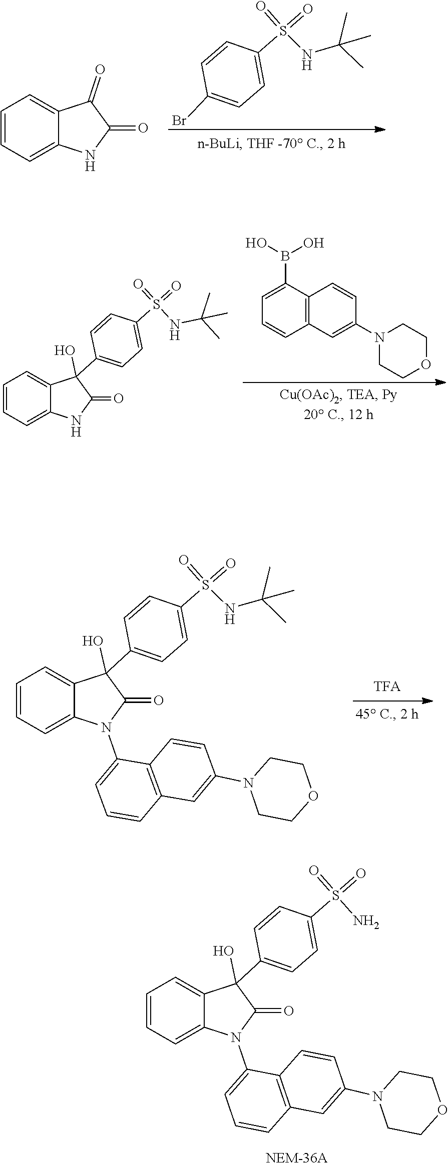

### 1. Synthesis of 1-benzyloxy-6-bromo-naphthalene

To a solution of 6-bromonaphthalen-1-ol (1 g, 4.48 mmol, 1 eq) and chloromethylbenzene (1.70 g, 13.45 mmol, 1.55 mL, 3 eq) in DMF (10 mL) was added K₂CO₃ (1.86 g, 13.45 mmol, 3 eq) and KI (74.42 mg, 448.30 umol, 0.1 eq). The mixture was stirred at 80° C. for 12 hr. The reaction mixture was cooled to room temperature and diluted by water (50 mL), extracted with ethyl acetate (50 mL*2). The combined organics were washed with brine (50 mL), dried over Na₂SO₄, filtered and concentrated under reduced pressure to give a residue. The residue was purified by flash column (ISCO 20 g silica, 0-20% ethyl acetate in petroleum ether gradient @40 mL/min). Compound 1-benzyloxy-6-bromo-naphthalene (1.3 g, 4.15 mmol, 92.59% yield) was obtained as a yellow oil.

### 2. Synthesis of 4-(5-benzyloxy-2-naphthyl)morpholine

A mixture of 1-benzyloxy-6-bromo-naphthalene (1.3 g, 4.15 mmol, 1 eq), morpholine (723.25 mg, 8.30 mmol, 730.55 uL, 2 eq), t-BuOK (931.55 mg, 8.30 mmol, 2 eq), palladium;tritert-butylphosphane (424.26 mg, 830.17 umol, 0.2 eq) in toluene (15 mL) was degassed and purged with N₂ for 3 times, and then the mixture was stirred at 110° C. for 3 hr under N₂ atmosphere. 20 mL of water was added to the mixture, the mixture was extracted with ethyl acetate (30 mL*2), and the combined extracts were washed with brine (20 mL), dried with anhydrous Na₂SO₄ and concentrated under reduced pressure to give a residue. The residue was purified by flash column (ISCO 20 g silica, 0-20% ethyl acetate in petroleum ether gradient @40 mL/min). Compound 4-(5-benzyloxy-2-naphthyl)morpholine (1 g, 3.13 mmol, 75.43% yield) was obtained as a yellow solid.

### 3. Synthesis of 6-morpholinonaphthalen-1-ol

To a solution of 4-(5-benzyloxy-2-naphthyl)morpholine (1 g, 3.13 mmol, 1 eq) in MeOH (15 mL) was added Pd/C (1 g, 10% purity) under N₂ atmosphere. The suspension was degassed and purged with H₂ for 3 times. The mixture was stirred under H₂ (15 Psi) at 20° C. for 12 hr. The suspension was filtered through a pad of Celite and the filter cake was washed with MeOH (20 mL×3). The filtrate was concentrated under reduced pressure to give a residue. Compound 6-morpholinonaphthalen-1-ol (600 mg, crude) was obtained as a gray solid which was used into next step without further purification.

### 4. Synthesis of (6-morpholino-1-naphthyl) trifluoromethanesulfonate

To a solution of 6-morpholinonaphthalen-1-ol (542.7 mg, 2.37 mmol, 1 eq) in DCM (5 mL) was added DIEA (1.22 g, 9.47 mmol, 1.65 mL, 4 eq) and Tf₂O (1.34 g, 4.73 mmol, 781.09 uL, 2 eq). The mixture was stirred at 20° C. for 2 hr. 15 mL of water was added to the mixture, the mixture was extracted with dichloromethane (15 mL*3), the combined extracts were washed with brine (10 mL), dried with anhydrous Na₂SO₄ and concentrated under reduced pressure to give a residue. The residue was purified by flash column (ISCO 20 g silica, 0-15% ethyl acetate in petroleum ether, gradient over 20 min). Compound (6-morpholino-1-naphthyl) trifluoromethanesulfonate (0.7288 g, 2.02 mmol, 85.21% yield) was obtained as a light yellow oil.

### 5. Synthesis of 4-[5-(4,4,5,5-tetramethyl-1,3,2-dioxaborolan-2-yl)-2-naphthyl]morpholine

A mixture of (6-morpholino-1-naphthyl) trifluoromethanesulfonate (700 mg, 1.94 mmol, 1 eq), 4,4,5,5-tetramethyl-2-(4,4,5,5-tetramethyl-1,3,2-dioxaborolan-2-yl)-1,3,2-dioxaborolane (1.97 g, 7.75 mmol, 4 eq), TEA (784.12 mg, 7.75 mmol, 1.08 mL, 4 eq), cyclopentyl(diphenyl)phosphane;dichloromethane;dichloropalladium;iron (158.20 mg, 193.73 umol, 0.1 eq) in dioxane (10 mL) was degassed and purged with N₂ for 3 times, and then the mixture was stirred at 90° C. for 12 hr under N₂ atmosphere. 10 mL of water was added to the mixture, the mixture was extracted with ethyl acetate (10 mL*3), and the combined extracts were washed with brine (30 mL), dried with anhydrous Na₂SO₄ and concentrated under reduced pressure to give a residue. The residue was purified by flash column (ISCO 20 g silica, 0-15% ethyl acetate in petroleum ether gradient @80 mL/min). Compound 4-[5-(4,4,5,5-tetramethyl-1,3,2-dioxaborolan-2-yl)-2-naphthyl]morpholine (700 mg, 1.24 mmol, 63.91% yield, 60% purity) was obtained as a light yellow oil. ¹H NMR (400 MHz, CHLOROFORM-d) δ 8.67 (d, J=9.3 Hz, 1H), 7.91 (dd, J=1.3, 6.9 Hz, 1H), 7.80 (d, J=8.1 Hz, 1H), 7.41 (dd, J=6.9, 8.1 Hz, 1H), 7.35-7.28 (m, 1H), 7.15-7.09 (m, 1H), 3.98-3.89 (m, 4H), 3.34-3.23 (m, 4H), 1.46-1.40 (s, 12H)

### 6. Synthesis of (6-morpholino-1-naphthyl)boronic acid

To a solution of 4-[5-(4,4,5,5-tetramethyl-1,3,2-dioxaborolan-2-yl)-2-naphthyl]morpholine (650 mg, 1.92 mmol, 1 eq) in THE (1 mL) and H₂O (0.3 mL) was added NaIO₄ (2.05 g, 9.58 mmol, 530.87 uL, 5 eq), NH₄OAc (147.70 mg, 1.92 mmol, 1 eq) in one portion at 20° C. for 12 h. The mixture was acidified by adding hydrochloric acid (1 M, 100 mL) dropwise at 0° C. to pH=6. The crude product was purified by prep-HPLC (column: Phenomenex C18 80*40 mm*3 um;mobile phase: [water(NH4HCO3)-ACN];B %: 5%-35%, 8 min). Compound (6-morpholino-1-naphthyl)boronic acid (20 mg, 77.79 umol, 4.06% yield) was obtained as a brown solid.

### 7. Synthesis of N-tert-butyl-4-(3-hydroxy-2-oxo-indolin-3-yl)benzenesulfonamide

To a solution of 4-bromo-N-tert-butyl-benzenesulfonamide (5.36 g, 18.35 mmol, 2 eq) in THE (30 mL) was added dropwise n-BuLi (2.5 M, 14.68 mL, 4 eq) at −70° C. over 5 min. After addition, the mixture was stirred at this temperature for 30 min, and then indoline-2,3-dione (1.35 g, 9.18 mmol, 1 eq) in THE (20 mL) was added dropwise at −70° C. The resulting mixture was stirred at −70° C. for 1.5 hr. The reaction was poured into ice NH₄C₁ aq. 50 mL at 0° C., the mixture was extracted with ethyl acetate (50 mL*3), and the combined extracts were washed with brine (50 mL), dried with anhydrous Na₂SO₄ and filtered. The filtrate was concentrated under reduced pressure to give a residue. The residue was purified by flash column (ISCO 12 g silica, 0˜100% ethyl acetate in petroleum ether gradient @40 mL/min). Compound N-tert-butyl-4-(3-hydroxy-2-oxo-indolin-3-yl)benzenesulfonamide (0.9 g, 2.50 mmol, 27.21% yield) was obtained as a yellow solid.

### 8. Synthesis of N-tert-butyl-4-[3-hydroxy-1-(6-morpholino-1-naphthyl)-2-oxo-indolin-3-yl]benzenesulfonamide

To a solution of N-tert-butyl-4-(3-hydroxy-2-oxo-indolin-3-yl)benzenesulfonamide (28 mg, 77.69 umol, 1 eq) and (6-morpholino-1-naphthyl)boronic acid (19.97 mg, 77.69 umol, 1 eq) in DCE (1 mL) was added Pyridine (6.14 mg, 77.69 umol, 6.27 uL, 1 eq), TEA (7.86 mg, 77.69 umol, 10.81 uL, 1 eq) and Cu(OAc)₂ (14.11 mg, 77.69 umol, 1 eq), the mixture was stirred at 20° C. for 12 hr under 02 atmosphere. The suspension was filtered through a pad of Celite and the filter cake was washed with dichloromethane (2 mL*3). The filtrate was concentrated under reduced pressure to give a residue. The residue was purified by flash column (ISCO 12 g silica, 0-50% ethyl acetate in petroleum ether gradient @40 mL/min). Compound N-tert-butyl-4-[3-hydroxy-1-(6-morpholino-1-naphthyl)-2-oxo-indolin-3-yl]benzenesulfonamide (10 mg, 10.50 umol, 13.51% yield, 60% purity) was obtained as a yellow solid.

### 9. Synthesis of 4-[3-hydroxy-1-(6-morpholino-1-naphthyl)-2-oxo-indolin-3-yl]benzenesulfonamide (NEM-36A)

To a solution of N-tert-butyl-4-[3-hydroxy-1-(6-morpholino-1-naphthyl)-2-oxo-indolin-3-yl]benzenesulfonamide (10 mg, 10.50 umol, 60% purity, 1 eq) in DCM (0.5 mL) was added dropwise TFA (385.00 mg, 3.38 mmol, 0.25 mL, 321.72 eq). The mixture was stirred at 45° C. for 2 hr. The reaction was concentrated under reduced pressure. The residue was purified by prep-HPLC (column: Phenomenex C18 75*30 mm*3 um;mobile phase: [water(NH4HCO3)-ACN];B %: 20%-50%, 8 min). Compound 4-[3-hydroxy-1-(6-morpholino-1-naphthyl)-2-oxo-indolin-3-yl]benzenesulfonamide (1.8 mg, 3.49 umol, 33.26% yield) was obtained as a white solid. ¹H NMR (400 MHz, ACETONITRILE-d3) δ 7.96-7.87 (m, 3H), 7.71 (dd, J=8.6, 10.9 Hz, 2H), 7.64-7.52 (m, 2H), 7.33 (br d, J=17.6 Hz, 6H), 7.21-7.11 (m, 1H), 6.47 (dd, J=7.9, 11.8 Hz, 1H), 5.70 (br d, J=6.0 Hz, 2H), 3.85-3.79 (m, 4H), 3.26 (td, J=5.0, 10.5 Hz, 4H). MS (M+H)+=515.95

### Example 29. Synthesis of Compound NEM-37A

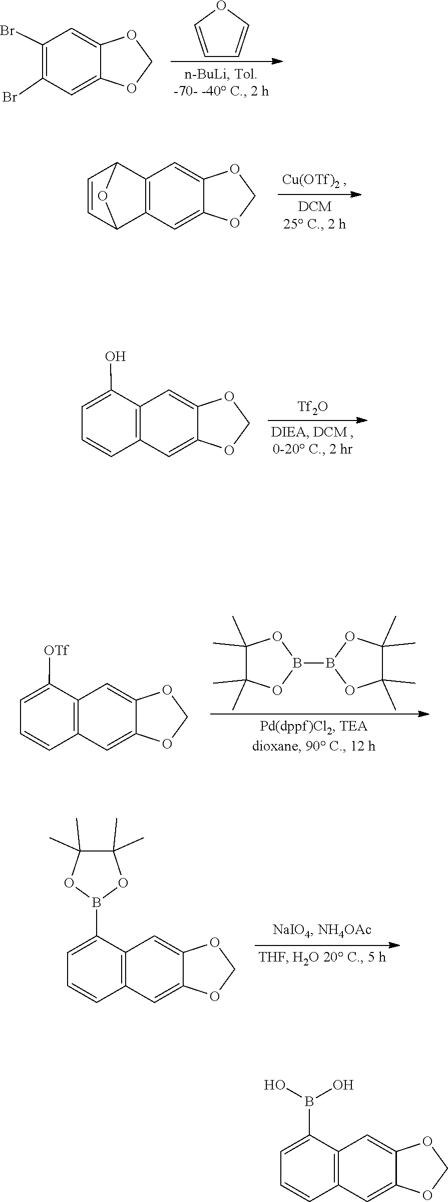

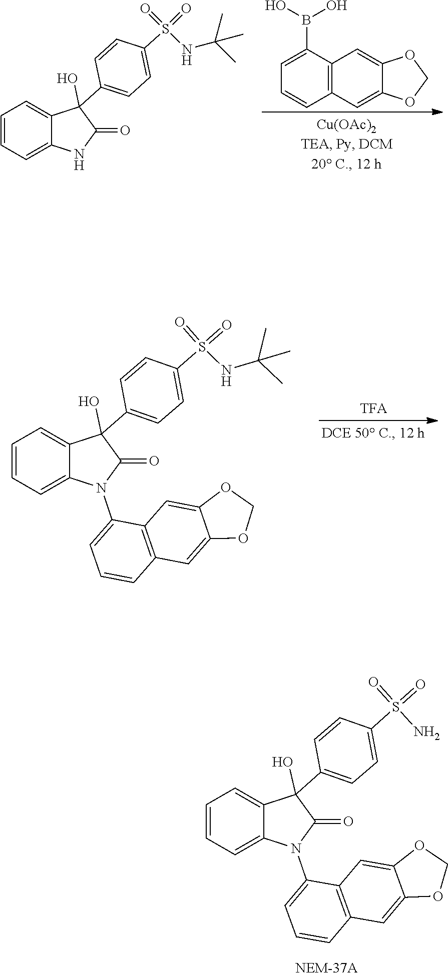

### 1. Synthesis of 5,7,14-trioxatetracyclo[9.2.1.02,10.04,8]tetradeca-2(10),3,8,12-tetraene

To a stirred solution of 5,6-dibromo-1,3-benzodioxole (2 g, 7.15 mmol, 308.64 uL, 1 eq) and freshly distilled furan (2.43 g, 35.73 mmol, 2.60 mL, 5 eq) in toluene (20 mL) was added dropwise n-BuLi (2 M, 4.29 mL, 1.2 eq) over 5 minutes at −70° C. The reaction mixture was stirred for 10 minutes before it was warmed up to −40° C. Stirring was continued for another 2 hours. The mixture was quenched with 1 N HCl aq. 10 mL, and then diluted with H₂O 100 mL and extracted with solvent ethyl acetate (50 mL*2). The combined organic layers were washed with brine, dried over Na₂SO₄ and concentrated under reduced pressure to give a residue. The crude product 5,7,14-trioxatetracyclo[9.2.1.02,10.04,8]tetradeca-2(10),3,8,12-tetraene (1 g, 4.25 mmol, 59.50% yield, 80% purity) was obtained as a white solid which was used into the next step without further purification.

### 2. Synthesis of benzo[f][1,3]benzodioxol-5-ol

To a solution of 5,7,14-trioxatetracyclo[9.2.1.02,10.04,8]tetradeca-2(10),3,8,12-tetraene (1.2 g, 6.38 mmol, 1 eq) in DCM (12 mL), bis(trifluoromethylsulfonyloxy)copper (115.32 mg, 318.85 umol, 0.050 eq) was added. The reaction mixture was degassed with N₂ for three times and at 25° C. stirred for 2 hr under N₂ atmosphere. 15 mL of water was added to the mixture, the mixture was extracted with DCM (15 mL*3), the combined extracts were washed with brine (10 mL), dried with anhydrous Na₂SO₄ and concentrated under reduced pressure to give a residue. Compound benzo[f][1,3]benzodioxol-5-ol (1.7 g, crude) was obtained as a light yellow oil.

### 3. Synthesis of benzo[f][1,3]benzodioxol-5-yl trifluoromethanesulfonate

To a solution of benzo[f][1,3]benzodioxol-5-ol (1.6 g, 8.50 mmol, 1 eq) in DCM (15 mL) was added DIEA (4.40 g, 34.01 mmol, 5.92 mL, 4 eq) and Tf₂O (4.80 g, 17.01 mmol, 2.81 mL, 2 eq) at 0° C. The mixture was stirred at 20° C. for 2 hr. 35 mL of water was added to the mixture, the mixture was extracted with DCM (25 mL*3), the combined extracts were washed with brine (20 mL), dried with anhydrous Na₂SO₄ and concentrated under reduced pressure to give a residue. The residue was purified by flash silica gel chromatography (ISCO; 25 g SepaFlash Silica Flash Column, Eluent of 0˜15% Ethyl acetate/Petroleum ether gradient @80 mL/min). Compound benzo[f][1,3]benzodioxol-5-yl trifluoromethanesulfonate (2.3 g, 7.18 mmol, 84.47% yield) was obtained as a light yellow oil. ¹H NMR (400 MHz, CHLOROFORM-d) δ 7.66-7.59 (m, 1H), 7.31-7.27 (m, 2H), 7.22 (s, 1H), 7.13 (s, 1H), 6.07 (s, 2H)

### 4. Synthesis of 2-benzo[f][1,3]benzodioxol-5-yl-4,4,5,5-tetramethyl-1,3,2-dioxaborolane

A mixture of benzo[f][1,3]benzodioxol-5-yl trifluoromethanesulfonate (1 g, 3.12 mmol, 1 eq) 4,4,5,5-tetramethyl-2-(4,4,5,5-tetramethyl-1,3,2-dioxaborolan-2-yl)-1,3,2-dioxaborolane (3.96 g, 15.61 mmol, 5 eq), TEA (1.26 g, 12.49 mmol, 1.74 mL, 4 eq), cyclopentyl(diphenyl)phosphane;dichloromethane;dichloropalladium;iron (255.01 mg, 312.26 umol, 0.1 eq) in dioxane (10 mL) was degassed and purged with N₂ for 3 times, and then the mixture was stirred at 90° C. for 12 hr under N₂ atmosphere. 10 mL of water was added to the mixture, the mixture was extracted with ethyl acetate (10 mL*3), and the combined extracts were washed with brine (10 mL), dried with anhydrous Na₂SO₄ and concentrated under reduced pressure to give a residue. The residue was purified by flash silica gel chromatography (ISCO; 25 g SepaFlash Silica Flash Column, Eluent of 0˜15% Ethyl acetate/Petroleum ether gradient @40 mL/min). Compound 2-benzo[f][1,3]benzodioxol-5-yl-4,4,5,5-tetramethyl-1,3,2-dioxaborolane (1.7 g, 2.85 mmol, 91.30% yield, 50% purity) was obtained as a white solid. ¹H NMR (400 MHz, CHLOROFORM-d) δ 8.19 (s, 1H), 7.93 (d, J=6.9 Hz, 1H), 7.76 (d, J=8.0 Hz, 1H), 7.36-7.30 (m, 1H), 7.14-7.09 (d, J=3.6 Hz, 1H), 6.04 (s, 2H), 1.41 (s, 12H).

### 5. Synthesis of benzo[f][1,3]benzodioxol-5-ylboronic acid

To a solution of 2-benzo[f][1,3]benzodioxol-5-yl-4,4,5,5-tetramethyl-1,3,2-dioxaborolane (1.5 g, 5.03 mmol, 1 eq) in THE (3 mL) and H₂O (1 mL) was added NaIO₄ (5.38 g, 25.16 mmol, 1.39 mL, 5 eq), NH₄OAc (581.72 mg, 7.55 mmol, 1.5 eq) in portion. The mixture was stirred at 20° C. for 5 hr. The mixture was acidified by adding hydrochloric acid (1 M, 10 mL) dropwise at 0° C. to pH=6. The reaction mixture was concentrated, then the reaction mixture was filtered and the filter cake was concentrated under reduced pressure. Compound benzo[f][1,3]benzodioxol-5-ylboronic acid (320 mg, crude) was obtained as a brown solid.

### 6. Synthesis of 4-(1-benzo[f][1,3]benzodioxol-5-yl-3-hydroxy-2-oxo-indolin-3-yl)-N-tert-butyl-benzenesulfonamide

A mixture of N-tert-butyl-4-(3-hydroxy-2-oxo-indolin-3-yl)benzenesulfonamide (150 mg, 416.17 umol, 1 eq), benzo[f][1,3]benzodioxol-5-ylboronic acid (179.78 mg, 832.35 umol, 2 eq), TEA (84.22 mg, 832.35 umol, 115.85 uL, 2 eq), Pyridine (65.84 mg, 832.35 umol, 67.18 uL, 2 eq) and Cu(OAc)₂ (151.18 mg, 832.35 umol, 2 eq) in DCM (2 mL) was degassed and purged with 02 for 3 times, and then the mixture was stirred at 20° C. for 12 hr under 02 atmosphere. 5 mL of water was added to the mixture, the mixture was extracted with DCM (5 mL*3), the combined extracts were washed with brine (10 mL), dried with anhydrous Na₂SO₄ and concentrated under reduced pressure to give a residue. The residue was purified by flash silica gel chromatography (ISCO; 25 g SepaFlash Silica Flash Column, Eluent of 0-100% Ethyl acetate/Petroleum ether gradient @40 mL/min). Compound 4-(1-benzo[f][1,3]benzodioxol-5-yl-3-hydroxy-2-oxo-indolin-3-yl)-N-tert-butyl-benzenesulfonamide (42 mg, 55.41 umol, 13.31% yield, 70% purity) was obtained as a light yellow oil.

### 7. 4-(1-benzo[f][1,3]benzodioxol-5-yl-3-hydroxy-2-oxo-indolin-3-yl)benzenesulfonamide (NEM-37A)

To a solution of 4-(1-benzo[f][1,3]benzodioxol-5-yl-3-hydroxy-2-oxo-indolin-3-yl)-N-tert-butyl-benzenesulfonamide (42 mg, 79.16 umol, 1 eq) in DCM (1 mL) was added TFA (3.08 g, 27.01 mmol, 2 mL, 341.25 eq). The mixture was stirred at 50° C. for 12 hr. The mixture was evaporated under reduced pressure. The residue was purified by preparative HPLC (column: Phenomenex Luna 80*30 mm*3 um;mobile phase: [water(TFA)-ACN];B %: 30%-60%, 8 min). Compound 4-(1-benzo[f][1,3]benzodioxol-5-yl-3-hydroxy-2-oxo-indolin-3-yl)benzenesulfonamide (3.5 mg, 7.38 mol, 9.32% yield, 100% purity) was obtained as a yellow solid. ¹H NMR (400 MHz, ACETONITRILE-d3) δ 7.94-7.85 (m, 3H), 7.70 (t, J=8.9 Hz, 2H), 7.55-7.46 (m, 1H), 7.45-7.41 (m, 1H), 7.39-7.24 (m, 3H), 7.22-7.11 (m, 1H), 7.05-7.01 (m, 0.5H), 6.74-6.70 (m, 0.5H), 6.51-6.45 (m, 1H), 6.09-6.00 (m, 2H), 5.74-5.62 (m, 2H). MS (M+H)⁺=475.0

### Example 30. Synthesis of Compound NEM-38A

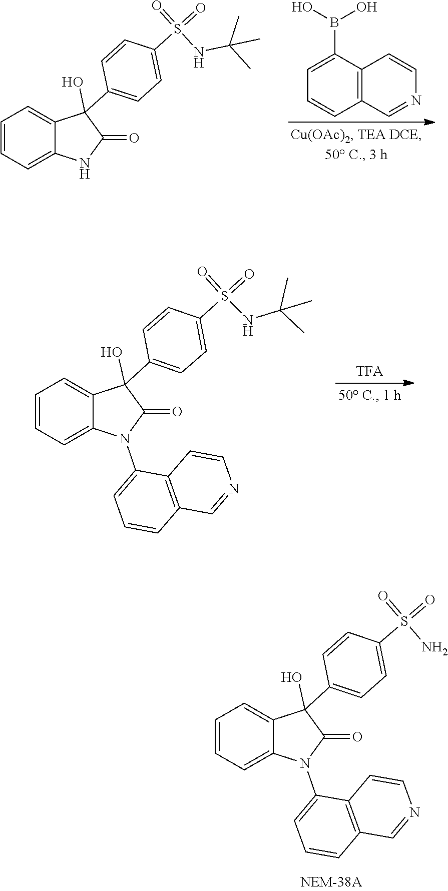 [SMILES: CC(C)(C)NS(=O)(=O)c1ccc(C2(O)C(=O)Nc3ccccc32)cc1 ; OB(O)c1cccc2cnccc12 ; CC(C)(C)NS(=O)(=O)c1ccc(C2(O)C(=O)N(c3cccc4cnccc34)c3ccccc32)cc1 ; NS(=O)(=O)c1ccc(C2(O)C(=O)N(c3cccc4cnccc34)c3ccccc32)cc1]

### 1. Synthesis of N-tert-butyl-4-[3-hydroxy-1-(5-isoquinolyl)-2-oxo-indolin-3-yl]benzenesulfonamide

A mixture of N-tert-butyl-4-(3-hydroxy-2-oxo-indolin-3-yl)benzenesulfonamide (200 mg, 554.90 umol, 1 eq), 5-isoquinolylboronic acid (287.95 mg, 1.66 mmol, 3 eq), Cu(OAc)₂ (201.57 mg, 1.11 mmol, 2 eq), TEA (112.30 mg, 1.11 mmol, 154.47 uL, 2 eq) and Pyridine (87.78 mg, 1.11 mmol, 89.58 uL, 2 eq) in DCM (5 mL) was degassed and purged with N₂ for 3 times, and then the mixture was stirred at 50° C. for 3 hr under N₂ atmosphere. 5 mL of water was added to the mixture, the mixture was extracted with DCM (20 mL*2), and the combined extracts were washed with brine (20 mL), dried with anhydrous Na₂SO₄ and concentrated under reduced pressure to give a residue. The residue was purified by prep-HPLC (column: Phenomenex C18 80*40 mm*3 um;mobile phase: [water(NH4HCO3)-ACN];B %: 30%-50%, 8 min). Compound N-tert-butyl-4-[3-hydroxy-1-(5-isoquinolyl)-2-oxo-indolin-3-yl]benzenesulfonamide (35 mg, 71.78 umol, 12.94% yield) was obtained as a yellow solid. MS (M+H)+=488.1

### 2. Synthesis of 4-[3-hydroxy-1-(5-isoquinolyl)-2-oxo-indolin-3-yl]benzenesulfonamide (NE M-38A)

To a solution of N-tert-butyl-4-[3-hydroxy-1-(5-isoquinolyl)-2-oxo-indolin-3-yl]benzenesulfonamide (30 mg, 61.53 umol, 1 eq) in DCM (1 mL) was added TFA (1.15 g, 10.13 mmol, 750.00 uL, 164.63 eq). The mixture was stirred at 50° C. for 1 hr. The reaction mixture was concentrated under reduced pressure to give a residue. The residue was purified by preparative HPLC (column: Phenomenex Luna C18 75*30 mm*3 um;mobile phase: [water(TFA)-ACN];B %: 10%-40%, 8 min). Compound 4-[3-hydroxy-1-(5-isoquinolyl)-2-oxo-indolin-3-yl]benzenesulfonamide (17.7 mg, 32.42 umol, 52.69% yield, TFA salt) was obtained as yellow solid. ¹H NMR (400 MHz, ACETONITRILE-d3) δ 9.58 (br d, J=6.8 Hz, 1H), 8.59-8.48 (m, 1H), 8.47-8.40 (m, 1H), 8.08 (br d, J=7.4 Hz, 1H), 8.01 (br t, J=8.1 Hz, 1H), 7.96-7.88 (m, 2H), 7.85 (br s, 0.5H), 7.76-7.69 (m, 2H), 7.64 (br s, 0.5H), 7.41-7.28 (m, 2H), 7.22 (dd, J=7.6, 11.1 Hz, 1H), 6.59-6.51 (m, 1H), 5.70 (br d, J=6.4 Hz, 2H). MS (M+H)+=432.0

### Example 31. Synthesis of Compound NEM-41A

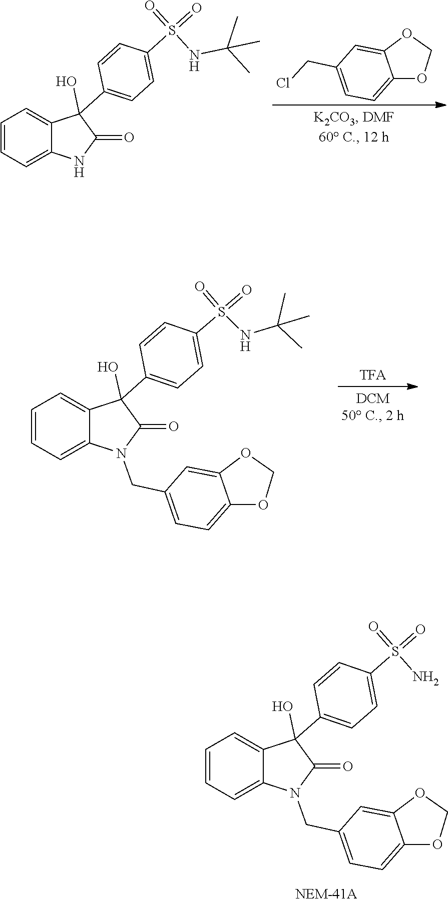

### 1. Synthesis of 4-[1-(1,3-benzodioxol-5-ylmethyl)-3-hydroxy-2-oxo-indolin-3-yl]-N-tert-buty 1-benzenesulfonamide

To a solution of N-tert-butyl-4-(3-hydroxy-2-oxo-indolin-3-yl)benzenesulfonamide (110 mg, 305.19 umol, 1 eq) in DMF (2 mL) was added K₂CO₃ (126.54 mg, 915.58 umol, 3 eq) and 5-(chloromethyl)-1,3-benzodioxole (52.06 mg, 305.19 umol, 1 eq), the mixture was stirred at 60° C. for 12 hr. 5 mL of water was added to the mixture, the mixture was extracted with ethyl acetate (20 mL*2), and the combined extracts were washed with brine (20 mL), dried with anhydrous Na₂SO₄ and concentrated under reduced pressure to give a residue. The residue was purified by flash column (ISCO 20 g silica, 0-60% ethyl acetate in petroleum ether, gradient over 20 min). Compound 4-[1-(1,3-benzodioxol-5-ylmethyl)-3-hydroxy-2-oxo-indolin-3-yl]-N-tert-butyl-benzenesulfonamide (60 mg, 121.32 umol, 39.75% yield) was obtained as a yellow solid.

### 2. Synthesis of 4-[1-(1,3-benzodioxol-5-ylmethyl)-3-hydroxy-2-oxo-indolin-3-yl]benzenesulfonamide (NEM-41A)

To a solution of 4-[1-(1,3-benzodioxol-5-ylmethyl)-3-hydroxy-2-oxo-indolin-3-yl]-N-tert-butyl-benzenesulfonamide (60 mg, 121.32 umol, 1 eq) in DCM (1 mL) was added TFA (660.00 mg, 5.79 mmol, 428.57 uL, 47.71 eq). The mixture was stirred at 50° C. for 2 hr. The mixture was concentrated under reduced pressure to give a residue. The residue was purified by preparative HPLC (column: Phenomenex Luna C18 80*30 mm*3 um;mobile phase: [water(TFA)-ACN];B %: 30%-60%, 8 min) to give desired compound. Compound 4-[1-(1,3-benzodioxol-5-ylmethyl)-3-hydroxy-2-oxo-indolin-3-yl]benzenesulfonamide (9.5 mg, 20.58 umol, 16.96% yield) was obtained as yellow solid. ¹H NMR (400 MHz, METHANOL-d4) δ 7.86 (d, J=8.4 Hz, 2H), 7.50 (d, J=8.4 Hz, 2H), 7.32 (dt, J=1.1, 7.7 Hz, 1H), 7.20 (d, J=7.0 Hz, 1H), 7.13-7.05 (m, 1H), 7.01 (d, J=7.9 Hz, 1H), 6.91-6.82 (m, 2H), 6.77 (d, J=7.9 Hz, 1H), 5.97-5.87 (d, J=1.6 Hz, 2H), 4.87 (s, 2H). MS (M+H)+=439.1

### Example 32. Synthesis of Compound NEM-45A

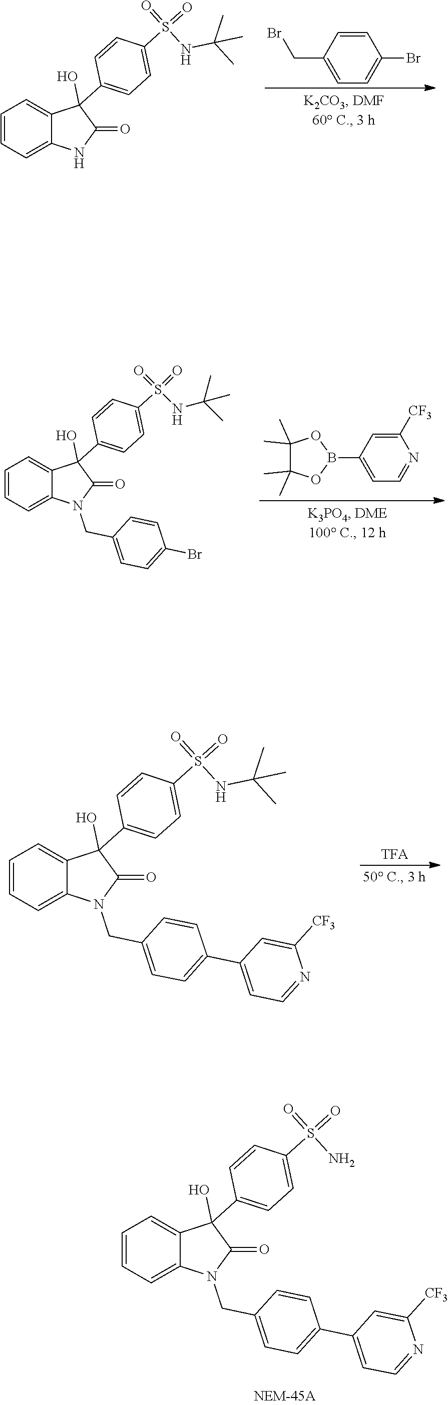

### 1. Synthesis of 4-[1-[(4-bromophenyl)methyl]-3-hydroxy-2-oxo-indolin-3-yl]-N-tert-butyl-benzenesulfonamide

To a solution of N-tert-butyl-4-(3-hydroxy-2-oxo-indolin-3-yl)benzenesulfonamide (200 mg, 554.90 mol, 1 eq) and K₂CO₃ (191.72 mg, 1.39 mmol, 2.5 eq) in DMF (2 mL) was added 1-bromo-4-(bromomethyl)benzene (166.42 mg, 665.88 mol, 1.2 eq). Then the mixture was stirred at 60° C. for 3 hr. 5 mL of water was added to the mixture, the mixture was extracted with ethyl acetate (10 mL*3) and the combined extracts were washed with brine (6 mL), dried with anhydrous Na₂SO₄ and concentrated under reduced pressure to give a residue. The residue was purified by flash silica gel chromatography (ISCO; 4 g SepaFlash Silica Flash Column, Eluent of 0˜100% Ethyl acetate/Petroleum ether gradient @50 mL/min). Compound 4-[1-[(4-bromophenyl)methyl]-3-hydroxy-2-oxo-indolin-3-yl]-N-tert-butyl-benzenesulfonamide (0.295 g, 501.47 mol, 90.37% yield, 90% purity) was obtained as a light yellow solid. MS (M+H)⁺=529.1

### 2. Synthesis of N-tert-butyl-4-[3-hydroxy-2-oxo-1-[[4-[2-(trifluoromethyl)-4-pyridyl]phenyl]methyl]indolin-3-yl]benzenesulfonamide

A mixture of 4-[1-[(4-bromophenyl)methyl]-3-hydroxy-2-oxo-indolin-3-yl]-N-tert-butyl-benzenesulfonamide (60 mg, 113.33 mol, 1 eq), 4-(4,4,5,5-tetramethyl-1,3,2-dioxaborolan-2-yl)-2-(trifluoromethyl)pyridine (34.04 mg, 124.66 mol, 1.1 eq), K₃PO₄ (48.11 mg, 226.65 mol, 2 eq), cyclopentyl(diphenyl)phosphane;dichloromethane; dichloropalladium;iron (18.51 mg, 22.67 mol, 0.2 eq) in DME (1 mL) was degassed and purged with N₂ for 3 times, and then the mixture was stirred at 100° C. for 12 hr under N₂ atmosphere. 5 mL of water was added to the mixture, the mixture was extracted with ethyl acetate (5 mL*3), and the combined extracts were washed with brine (5 mL), dried with anhydrous Na₂SO₄ and concentrated under reduced pressure to give a residue. The residue was purified by flash silica gel chromatography (ISCO; 12 g SepaFlash Silica Flash Column, Eluent of 0˜100% Ethyl acetate/Petroleum ether gradient @40 mL/min). Compound N-tert-butyl-4-[3-hydroxy-2-oxo-1-[[4-[2-(trifluoromethyl)-4-pyridyl]phenyl]methyl]indolin-3-yl]benzenesulfonamide (50 mg, 83.94 mol, 74.07% yield) was obtained as a light yellow solid. MS (M+H)⁺=596.3

### 3. 4-[3-hydroxy-2-oxo-1-[[4-[2-(trifluoromethyl)-4-pyridyl]phenyl]methyl]indolin-3-yl]benzenesulfonamide (NEM-45A)

To a solution of N-tert-butyl-4-[3-hydroxy-2-oxo-1-[[4-[2-(trifluoromethyl)-4-pyridyl]phenyl]methyl]indolin-3-yl]benzenesulfonamide (50 mg, 83.94 mol, 1 eq) in DCM (1 mL) was added TFA (3.07 g, 26.92 mmol, 2 mL, 320.74 eq). Then the mixture was stirred at 50° C. for 3 h. The mixture was evaporated under reduced pressure. The residue was purified by preparative HPLC (column: Waters Xbridge BEH C18 100*30 mm*10 um;mobile phase: [water(NH4HCO3)-ACN];gradient:30%-65% B over 8 min). Compound 4-[3-hydroxy-2-oxo-1-[[4-[2-(trifluoromethyl)-4-pyridyl]phenyl]methyl]indolin-3-yl]benzenesulfonamide (5 mg, 9.27 mol, 15.46% yield) was obtained as a white solid. ¹H NMR (400 MHz, ACETONITRILE-d3) δ 8.76 (d, J=5.1 Hz, 1H), 8.03 (s, 1H), 7.89-7.83 (m, 3H), 7.79 (d, J=8.4 Hz, 2H), 7.56-7.50 (m, 4H), 7.33 (dt, J=1.3, 7.8 Hz, 1H), 7.24-7.21 (m, 1H), 7.09 (dt, J=0.8, 7.5 Hz, 1H), 6.98 (d, J=7.9 Hz, 1H), 5.65 (br s, 2H), 5.01 (d, J=4.4 Hz, 2H), 4.81 (s, 1H). MS (M+H)⁺=540.2

### Biological Example 1. Biological Evaluation

To evaluate 4-(3-hydroxy-1-(4-methoxybenzyl)-2-oxoindolin-3-yl)benzenesulfonamide (Examples 1 and 2) for its ability to effectively inhibit Car1 enzyme activity, stopped-flow assays were performed, and its half-maximal inhibitory concentration (IC₅₀) was determined and compared to the known Car enzyme inhibitor MZ. Employing the stopped-flow assay, it was determined that 4-(3-hydroxy-1-(4-methoxybenzyl)-2-oxoindolin-3-yl)benzenesulfonamide is more potent at inhibiting Car1 than the established inhibitor MZ (Table 1). These results strongly suggested that 4-(3-hydroxy-1-(4-methoxybenzyl)-2-oxoindolin-3-yl)benzenesulfonamide will be much more efficient at inhibiting mast cell development than MZ and thereby possesses substantial therapeutic potential.

TABLE 1
IC₅₀s of Car enzyme inhibitors

| Compounds | IC₅₀ (nM) |
|---|---|
| MZ | 40.33 |
| 4-(3-hydroxy-1-(4-methoxybenzyl)-2- | 0.044 |
| oxoindolin-3-yl)benzenesulfonamide (CAR0037) | |

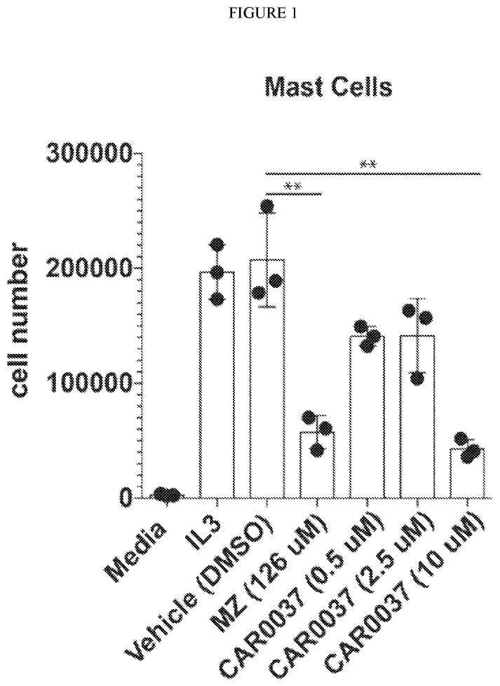

To further test the ability of 4-(3-hydroxy-1-(4-methoxybenzyl)-2-oxoindolin-3-yl)benzenesulfonamide to inhibit mast cell development, an established mast cell culture assay was employed (Henry, E. K., et al., J Exp Med, 2016. 213(9): p. 1663-73). As a positive control for mast cell development, bone marrow cells were isolated from the femur of mice and cultured in the presence of 10 ng/mL of IL-3 and vehicle (1:10, DMSO/RPMI complete media) for seven days and mast cells were identified by flow cytometric analysis as (c-Kit+ FceRIa+ CD49b− cells). To determine mast cell inhibitory capacity, cultures were treated with increasing doses of 4-(3-hydroxy-1-(4-methoxybenzyl)-2-oxoindolin-3-yl)benzenesulfonamide (CAR0037) (0.5 uM, 2.5 uM and 10 uM) and its effects were compared to MZ, which was used at 30 μg/mL (126.97 uM) (its peak effective range based on a previous study (Henry, E. K., et al., J Exp Med, 2016. 213(9): p. 1663-73)). Treatment with IL-3 resulted in increased mast cell numbers compared to media treated controls (FIG. 1). Further, while vehicle alone showed no effects, treatment with MZ resulted in significantly decreased mast cell numbers as previously reported (FIG. 1) (Henry, E. K., et al., J Exp Med, 2016. 213(9): p. 1663-73). Importantly, 10 uM of 4-(3-hydroxy-1-(4-methoxybenzyl)-2-oxoindolin-3-yl)benzenesulfonamide inhibited mast cell development as effectively as 126.97 uM of MZ. These data are consistent with our stopped-flow analysis (Table 1) and demonstrate that 4-(3-hydroxy-1-(4-methoxybenzyl)-2-oxoindolin-3-yl)benzenesulfonamide is a more potent inhibitor of in vitro mast cell responses than MZ.

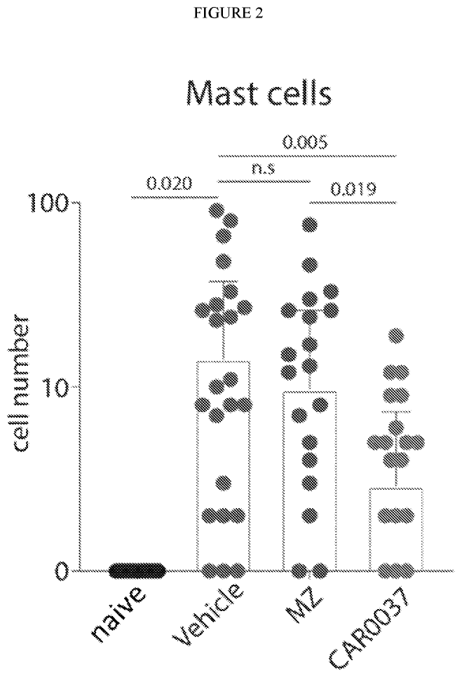

The stopped-flow and in vitro studies indicate that the novel compound 4-(3-hydroxy-1-(4-methoxybenzyl)-2-oxoindolin-3-yl)benzenesulfonamide is a more potent inhibitor of Car1 than MZ, and as a result, operates as a more efficient inhibitor of mast cell development. However, whether 4-(3-hydroxy-1-(4-methoxybenzyl)-2-oxoindolin-3-yl)benzenesulfonamide (CAR0037) is also more potent than MZ at inhibiting in vivo mast cell responses remained unknown. To address this, a model of Trichinella spiralis-induced mastocytosis was employed and the capacity of MZ and 4-(3-hydroxy-1-(4-methoxybenzyl)-2-oxoindolin-3-yl)benzenesulfonamide to inhibit intestinal mast cell responses was directly compared (Henry, E. K., et al., J Exp Med, 2016. 213(9): p. 1663-73). Briefly, mice were infected with T. spiralis and treated with either vehicle, MZ or 4-(3-hydroxy-1-(4-methoxybenzyl)-2-oxoindolin-3-yl)benzenesulfonamide and intestinal mast cells were quantified via esterase staining and histological analysis (Henry, E. K., et al., J Exp Med, 2016. 213(9): p. 1663-73). As reported previously, mice infected with T. spiralis exhibit significantly increased mast cell responses in the presence of vehicle (Henry, E. K., et al., J Exp Med, 2016. 213(9): p. 1663-73). Importantly, mice treated with MZ at 2 mg/day, a dose lower than what was required to inhibit mast cell responses in previous published studies (Henry, E. K., et al., J Exp Med, 2016. 213(9): p. 1663-73), showed no changes in mast cell responses post-infection (FIG. 2). In contrast, mice treated with 2 mg/day of 4-(3-hydroxy-1-(4-methoxybenzyl)-2-oxoindolin-3-yl)benzenesulfonamide presented with significantly decreased intestinal mast cell numbers (FIG. 2). These data are consistent with the in vitro assays and further demonstrate that 4-(3-hydroxy-1-(4-methoxybenzyl)-2-oxoindolin-3-yl)benzenesulfonamide is a more potent inhibitor of mast cells than MZ, and therefore, possesses substantial therapeutic potential.

To evaluate the ability of representative compounds of the invention to effectively inhibit mast cell development, in vitro mast cell cultures were performed as previously described (Henry et al), and the percent inhibition at 10 μm was measured. Results are shown in the following table.

| | Percent |
|---|---|
| Compound | Inhibition at 10 μm |
| 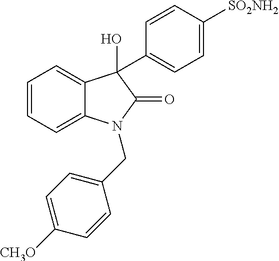 | 91 |
| 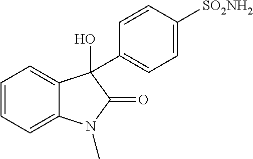 [SMILES: CN1C(=O)C(O)(c2ccc(S(N)(=O)=O)cc2)c2ccccc21] | 84 |
| 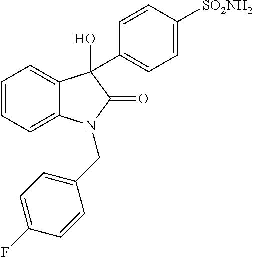 [SMILES: NS(=O)(=O)c1ccc(C2(O)C(=O)N(Cc3ccc(F)cc3)c3ccccc32)cc1] | 98 |
| 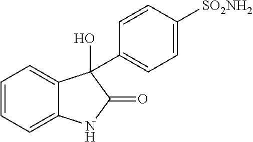 [SMILES: NS(=O)(=O)c1ccc(C2(O)C(=O)Nc3ccccc32)cc1] | 94 |
| 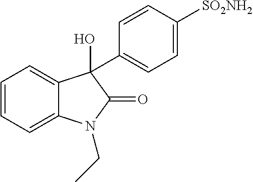 [SMILES: CCN1C(=O)C(O)(c2ccc(S(N)(=O)=O)cc2)c2ccccc21] | 99 |
| 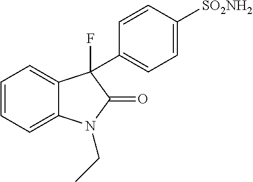 [SMILES: CCN1C(=O)C(F)(c2ccc(S(N)(=O)=O)cc2)c2ccccc21] | 99 |
| 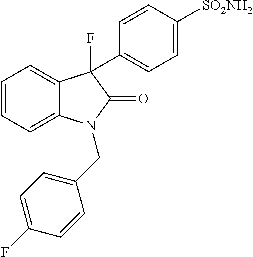 [SMILES: NS(=O)(=O)c1ccc(C2(F)C(=O)N(Cc3ccc(F)cc3)c3ccccc32)cc1] | 98 |
| 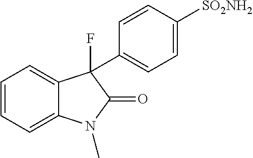 [SMILES: CN1C(=O)C(F)(c2ccc(S(N)(=O)=O)cc2)c2ccccc21] | 100 |
| 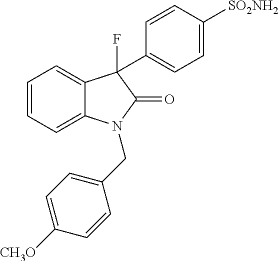 | 99 |

Additional compounds were evaluated with the stopped-flow assay, and the following table shows the IC₅₀ values of a few representative compounds:

TABLE 2
IC₅₀s of further Car enzyme inhibitors*

| Compounds | IC50 (nM) |
|---|---|
| NEM-9A | 51.3 |
| NEM-10A | 33.8 |
| NEM-12A | 62.5 |
| NEM-14A | 34.0 |
| NEM-15A | 35.3 |
| NEM-23A | 77 |
| NEM-24A | 44.3 |
| NEM-37A | 5.3 |

*The control, MZ, used in these assays shows an IC₅₀ of about 26-39 nM.

In additional, representative compounds were tested in the mast cell culture assay, and the results were shown in percent of inhibition (POI) based on MC numbers. Overall, all tested compounds were shown to have low/no toxicity based on viability observations.

TABLE 3
POI of further Car enzyme inhibitors at 15 uM*

| Compounds | POI |
|---|---|
| NEM-4A | 82 |
| NEM-9A | 18 |
| NEM-10A | 11 |
| NEM-12A | 6 |
| NEM-13A | 87 |
| NEM-14A | 98 |
| NEM-15A | 100 |
| NEM-19A | 99 |
| NEM-20A | 100 |
| NEM-21A | 81 |
| NEM-22A | 53 |
| NEM-23A | 99 |
| NEM-24A | 99 |
| NEM-25A | 87 |
| NEM-26A | 82 |
| NEM-27A | 94 |
| NEM-30A | 100 |
| NEM-32A | 100 |
| NEM-34A | 99 |
| NEM-35A | 99 |
| NEM-36A | 91 |
| NEM-37A | 100 |
| NEM-38A | 63 |
| NEM-41A | 64 |
| NEM-45A | 100 |

*The control, MZ, at 126 uM, used in these assays shows a POI ranging from about 78% up to 100%. As used herein, “uM” refers to micromolar or μM.

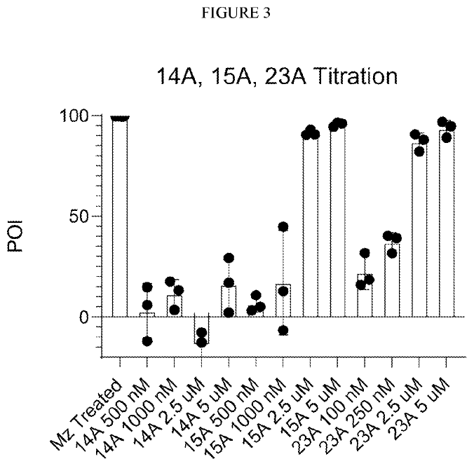

The POI data further shows that NEM-14A at 10 uM has a comparable inhibition of mast cell numbers with the control, MZ, at 126 uM. NEM-14A at 15 uM shows a significantly higher inhibition than MZ at 126 uM, with p=0.0046 (Student's t-test). The POI data also shows that NEM-15A at both 10 uM and 15 uM shows a significantly higher inhibition of mast cell numbers than MZ at 126 uM, with p=0.02 and 0.0049, respectively, Student's t-test. Additionally, NEM-23A was found to inhibit mast cell numbers at 5 uM, 10 uM, and 15 uM, all at comparable level than MZ at 126 uM. Further titration shows that NEM-23A inhibits mast cell numbers with close to 50% at 250 nM, and inhibits mast cell numbers even at 100 nM, see FIG. 3.

### Discussion

As new technologies and techniques are developed, our ability to understand hematopoiesis and lineage commitment events continues to advance. As a result, recent studies are beginning to reshape traditional models of stem cell development and are identifying previously unappreciated therapeutic targets. Previous studies identified a mast cell progenitor that is defined by its high expression of the enzyme Car1 (Henry, E. K., et al., J Exp Med, 2016. 213(9): p. 1663-73; Inclan-Rico, J. M., et al., PLoS Pathogens, 2020. 16(5): p. e1008579). These studies also demonstrated that inhibition of Car1 is sufficient to prevent mast cell development. Determining the developmental check points of mast cells is of great scientific and clinical relevance. Mast cells are strategically positioned at barrier surfaces and are robust producers of inflammatory molecules in response to diverse stimuli (Voehringer, D., Nat Rev Immunol, 2013. 13(5): p. 362-75). As a result, mast cells are critical players in several disease states including allergies, asthma, mastocytosis, mast cell activation syndrome and deadly anaphylaxis reactions (Henry, E. K., et al., J Exp Med, 2016. 213(9): p. 1663-73; Voehringer, D., Nat Rev Immunol, 2013. 13(5): p. 362-75). Collectively, these studies suggest that Car1 inhibitors can be employed to treat several forms of mast cell-mediated inflammation.

Previous studies have demonstrated that the Car enzyme inhibitor MZ can prevent both murine and human mast cell development. The studies presented here describe the design and synthesis of 4-(3-hydroxy-1-(4-methoxybenzyl)-2-oxoindolin-3-yl)benzenesulfonamide (CAR0037), a Car1 inhibitor that is much more potent in its ability to inhibit Car1 than MZ. Importantly, 4-(3-hydroxy-1-(4-methoxybenzyl)-2-oxoindolin-3-yl)benzenesulfonamide's capacity to inhibit Car1 correlated with its potent potential to inhibit mast cell responses both in vitro and in vivo. Collectively, these studies demonstrate that the Car1 inhibitor 4-(3-hydroxy-1-(4-methoxybenzyl)-2-oxoindolin-3-yl)benzenesulfonamide outcompetes MZ in both its ability to inhibit Car1 enzyme activation and mast cell development and possesses substantial therapeutic potential to treat mast cell-mediated diseases. Further studies show that compounds such as NEM-14A, 15-A, and 23-A are effective in inhibiting Car1 enzyme activation and can inhibit mast cell development at lower doses.

### Materials and Methods

Stopped-flow: IC₅₀ values were established using an SX20 stopped-flow spectrometer from Applied Biophysics. In one syringe of the SX20, recombinant human Car1 was kept at a concentration of 9 μM in a sodium phosphate buffer and inhibitors were added to this solution at 0 nM, 10 nM, 20 nM, 80 nM and 100 nM. In a second syringe, a saturated CO₂ solution was mixed with 0.2 mM phenol red. This series of dilutions allow for the evaluation of how quickly Car1 can metabolize the CO₂ in the absence of an inhibitor and for the determination of whether that process is inhibited as increasing concentrations of compounds are added.

In vitro Mast Cell development: Briefly, 1×10⁶ bone marrow-resident progenitor cells were isolated from the femur of mice and cultured in complete media supplemented with 10 ng/mL of rIL-3 for a period of 5 days. Mast cells were identified by their expression of the surface molecules FcεRIα and c-Kit via flow cytometric analysis. In addition to IL-3, cultures were treated with vehicle or increasing concentrations of Car enzyme inhibitors at the indicated concentrations.

Trichinella spiralis infection: Mice were infected with 500 T. spiralis muscle stage larvae by oral gavage and were treated with vehicle (1:5, DMSO/PBS), 2 mg MZ, or 2 mg 4-(3-hydroxy-1-(4-methoxybenzyl)-2-oxoindolin-3-yl)benzenesulfonamide (CAR0037) i.p. daily; mice were sacrificed between on day 10-post infection and mast cell responses in the small intestine were evaluated by esterase staining as described previously (Henry, E. K., et al., J Exp Med, 2016. 213(9): p. 1663-73).

The following illustrate representative pharmaceutical dosage forms, containing a compound of Formula I or Formula A (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

The invention has been described with reference to various specific and preferred embodiments and techniques. However, it should be understood that many variations and modifications may be made while remaining within the spirit and scope of the invention.

1. A compound of Formula A, or a pharmaceutically acceptable salt thereof: 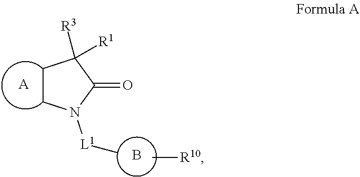
wherein:
R¹ is aryl, 5-membered heteroaryl, 6-membered heteroaryl, or (C₁-C₃)alkyl that is substituted with aryl, 5-membered heteroaryl, or 6-membered heteroaryl, wherein any aryl, 5-membered heteroaryl, and 6-membered heteroaryl, is substituted with —S(═O)₂NH₂ and is also optionally substituted with one or more groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{a}R^{b}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;
R³ is H, fluoro, hydroxy, (C₁-C₆)alkyl, or (C₁-C₆)alkoxy, wherein any (C₁-C₆)alkyl and (C₁-C₆)alkoxy, is optionally substituted with one or more fluoro;
Ring A is phenyl, 5-membered heteroaryl, or 6-membered heteroaryl, and ring A is optionally substituted, as valency permits, with 1, 2, 3, or 4 groups independently selected from the group consisting of halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy;
Ring B is absent, an optionally substituted carbocyclylene, optionally substituted arylene, optionally substituted heteroarylene, or optionally substituted heterocyclylene;
L¹ is absent or an optionally substituted C₁₋₃ alkylene;
R¹⁰ is hydrogen, halo, hydroxy, cyano, nitro, NH₂, COOH, CONH₂, S(O)₂NH₂, G¹, OG¹, NHG¹, NG¹G¹, C(O)G¹, C(O)OG¹, C(O)NHG¹, C(O)NG¹G¹, S(O)₂G¹, S(O)₂NHG¹, or S(O)₂NG¹G¹, wherein G¹ at each occurrence is independently an optionally substituted (C₁-C₆)alkyl, optionally substituted (C₁-C₆)heteroalkyl, optionally substituted 3-7 membered carbocyclic, optionally substituted aryl, optionally substituted heteroaryl, or optionally substituted heterocyclyl,
each R^{a} and R^{b} is independently selected from the group consisting of H, (C₁-C₆)alkyl, (C₃-C₆)cycloalkyl, and (C₃-C₆)cycloalkyl(C₁-C₆)alkyl; or R^{a} and R^{b} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl; and
each R^{e} and R^{f} is independently selected from the group consisting of H, (C₁-C₆)alkyl, (C₃-C₆)cycloalkyl, and (C₃-C₆)cycloalkyl(C₁-C₆)alkyl; or R^{e} and R^{f} together with the nitrogen to which they are attached form an aziridino, azetidino, morpholino, piperazino, pyrrolidino or piperidino, which aziridino, azetidino, morpholino, piperazino, pyrrolidino and piperidino is optionally substituted with one or more groups independently selected from the group consisting of halo and (C₁-C₆)alkyl.

2. The compound of claim 1, or a pharmaceutically acceptable salt thereof, characterized as having a structure according to Formula A-1: 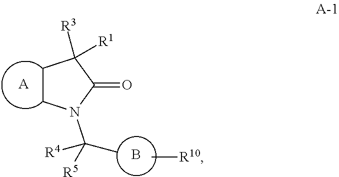
wherein: R⁴ and R⁵ are each independently hydrogen or an optionally substituted C₁₋₃ alkyl.

3. The compound of claim 2, or a pharmaceutically acceptable salt thereof, wherein ring B is an optionally substituted phenylene or optionally substituted 5 or 6-membered heteroarylene.

4. The compound of claim 2, or a pharmaceutically acceptable salt thereof, wherein ring B is unsubstituted phenylene.

5. The compound of claim 2, or a pharmaceutically acceptable salt thereof, characterized as having a structure according to Formula A-1-A: 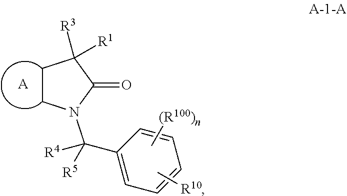
wherein:
n is 0, 1, 2, or 3; and
(i) R¹⁰⁰ at each occurrence is independently halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy, wherein R^{e} and R^{f} are defined in claim 1; or
(ii) R¹⁰⁰ at each occurrence is independently halogen, OH, C₁₋₄ alkyl, C₁₋₄ alkoxy, or C₁₋₄ heteroalkyl, wherein the C₁₋₄ alkyl, C₁₋₄ alkoxy, or C₁₋₄ heteroalkyl is optionally substituted with 1-3 fluorine; or
(iii) two adjacent R¹⁰⁰, together with the intervening atoms, are joined to form an optionally substituted 4-7 membered ring, which optionally contains a ring heteroatom and is aromatic or nonaromatic and any remaining R¹⁰⁰ is defined in (i) or (ii).

6. (canceled)

7. The compound of claim 2, or a pharmaceutically acceptable salt thereof, wherein R¹⁰ is an optionally substituted 4-7 membered monocyclic heterocyclic ring having 1 or 2 ring heteroatoms, wherein each ring heteroatom is independently nitrogen, oxygen, or sulfur, wherein when substituted, the 4-7 membered monocyclic heterocyclic ring is preferably substituted with 1-3 substituents, such as 1-3 substituents each independently selected from oxo, halogen (e.g., F), OH, C₁₋₄ alkyl, C₁₋₄ alkoxy, C₁₋₄ heteroalkyl, a 3-6 membered ring, or a nitrogen protecting group, wherein the C₁₋₄ alkyl, C₁₋₄ alkoxy, or C₁₋₄ heteroalkyl is optionally substituted with 1-3 fluorine.

8. The compound of claim 2, or a pharmaceutically acceptable salt thereof, wherein R¹⁰ is an optionally substituted 5 or 6 membered monocyclic heterocyclic ring having 1 or 2 ring heteroatoms, wherein each ring heteroatom is independently nitrogen, oxygen, or sulfur, such as pyrrolidine, piperidine, piperazine, morpholine, etc., wherein when substituted, the 5 or 6 membered monocyclic heterocyclic ring is preferably substituted with 1-3 substituents, such as 1-3 substituents each independently selected from oxo, halogen (e.g., F), OH, C₁₋₄ alkyl, C₁₋₄ alkoxy, C₁₋₄ heteroalkyl, a 3-6 membered ring, or a nitrogen protecting group, wherein the C₁₋₄ alkyl, C₁₋₄ alkoxy, or C₁₋₄ heteroalkyl is optionally substituted with 1-3 fluorine.

9. The compound of claim 2, or a pharmaceutically acceptable salt thereof, wherein R¹⁰ is an optionally substituted 5 or 6 membered heteroaryl ring having 1-4 ring heteroatoms, wherein each ring heteroatom is independently nitrogen, oxygen, or sulfur, such as pyridyl or imidazole ring, wherein a ring nitrogen atom is optionally oxidized, wherein when substituted, the 5 or 6 membered heteroaryl ring is preferably substituted with 1-3 substituents as valency permits, such as 1-3 substituents each independently selected from halogen (e.g., F), OH, C₁₋₄ alkyl, C₁₋₄ alkoxy, C₁₋₄ heteroalkyl, or a 3-6 membered ring, wherein the C₁₋₄ alkyl, C₁₋₄ alkoxy, or C₁₋₄ heteroalkyl is optionally substituted with 1-3 fluorine.

10. The compound of claim 2, or a pharmaceutically acceptable salt thereof, wherein R¹⁰ is selected from:

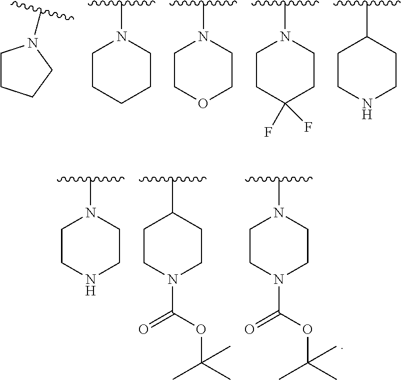

11. (canceled)

12. The compound of claim 2, or a pharmaceutically acceptable salt thereof, wherein ring B is an optionally substituted 5 or 6 membered heteroarylene having 1-4 ring heteroatoms, wherein each ring heteroatom is independently nitrogen, oxygen, or sulfur, such as pyridylene, wherein a ring nitrogen atom is optionally oxidized, wherein when substituted, the 5 or 6 membered heteroarylene is preferably substituted with 1-3 substituents as valency permits, such as 1-3 substituents each independently selected from halogen (e.g., F), OH, C₁₋₄ alkyl, C₁₋₄ alkoxy, C₁₋₄ heteroalkyl, or a 3-6 membered ring, wherein the C₁₋₄ alkyl, C₁₋₄ alkoxy, or C₁₋₄ heteroalkyl is optionally substituted with 1-3 fluorine.

13. The compound of claim 2, or a pharmaceutically acceptable salt thereof, wherein ring B is an optionally substituted bicyclic heteroarylene having 1-4 ring heteroatoms, wherein each ring heteroatom is independently nitrogen, oxygen, or sulfur, such as a 6,6-bicyclic heteroaryl ring, e.g., a quinoline or isoquinoline ring, wherein a ring nitrogen atom is optionally oxidized, wherein when substituted, the bicyclic heteroarylene is preferably substituted with 1-3 substituents as valency permits, such as 1-3 substituents each independently selected from halogen (e.g., F), OH, C₁₋₄ alkyl, C₁₋₄ alkoxy, C₁₋₄ heteroalkyl, or a 3-6 membered ring, wherein the C₁₋₄ alkyl, C₁₋₄ alkoxy, or C₁₋₄ heteroalkyl is optionally substituted with 1-3 fluorine.

14. The compound of claim 2, or a pharmaceutically acceptable salt thereof, wherein ring B is an optionally substituted 4-7 membered monocyclic heterocyclic ring having 1 or 2 ring heteroatoms, wherein each ring heteroatom is independently nitrogen, oxygen, or sulfur, such as a tetrahydropyran ring, etc., wherein when substituted, the 4-7 membered monocyclic heterocyclic ring is preferably substituted with 1-3 substituents, such as 1-3 substituents each independently selected from oxo, halogen (e.g., F), OH, C₁₋₄ alkyl, C₁₋₄ alkoxy, or C₁₋₄ heteroalkyl, or a nitrogen protecting group, wherein the C₁₋₄ alkyl, C₁₋₄ alkoxy, or C₁₋₄ heteroalkyl is optionally substituted with 1-3 fluorine.

15-17. (canceled)

18. The compound of claim 1, or a pharmaceutically acceptable salt thereof, characterized as having a structure according to Formula A-2:

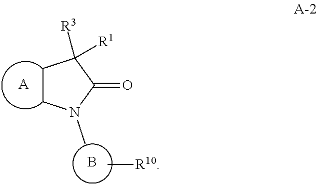

19. (canceled)

20. The compound of claim 18, or a pharmaceutically acceptable salt thereof, characterized as having a structure according to Formula A-2-A, A-2-B, or A-2-C: 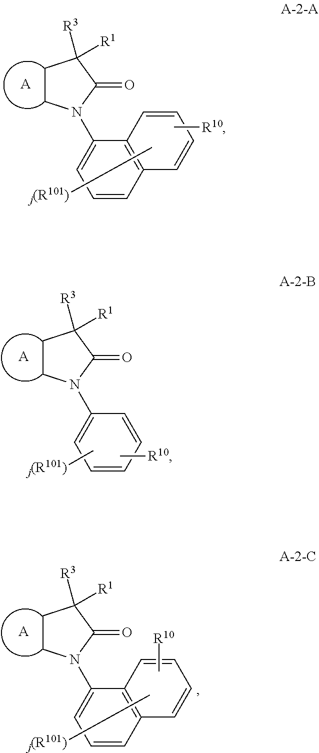
wherein:
j is 0, 1, 2, 3, or 4; and
(i) R¹⁰¹ at each occurrence is independently halo, hydroxy, cyano, nitro, (C₁-C₆)alkyl, (C₃-C₆)cycloalkyl, (C₁-C₆)alkoxy, (C₁-C₆)alkanoyl, (C₁-C₆)alkoxycarbonyl, (C₁-C₆)alkanoyloxy, and NR^{e}R^{f}, wherein any (C₁-C₆)alkyl, (C₃-C₆)cycloalkyl, (C₁-C₆)alkoxy, (C₁-C₆)alkanoyl, (C₁-C₆)alkoxycarbonyl, and (C₁-C₆)alkanoyloxy is optionally substituted with one or more groups independently selected from the group consisting of halo, hydroxy, cyano, nitro, (C₃-C₆)cycloalkyl, and (C₁-C₆)alkoxy, wherein R^{e} and R^{f} are defined in claim 1; or
(ii) R¹⁰¹ at each occurrence is independently halogen (e.g., F, Cl, Br, etc.), CN, OH, G², or OG², wherein G² at each occurrence is independently C₁₋₄ alkyl, C₁₋₄ heteroalkyl, 3-6 membered ring, (C₁₋₄ alkylene)-(3-6 membered ring), or (C₁₋₄ heteroalkylene)-(3-6 membered ring), wherein the C₁₋₄ alkyl, C₁₋₄ heteroalkyl, C₁₋₄ alkylene, or C₁₋₄ heteroalkylene, is optionally substituted with 1-3 fluorine; and the 3-6 membered ring is optionally substituted with halogen, CN, C₁₋₄ alkyl optionally substituted with 1-3 F, C₁₋₄ alkoxy optionally substituted with 1-3 F, or C₁₋₄ heteroalkyl optionally substituted with 1-3 F, or
(iii) two instances of R¹⁰¹ are joined together to form a 5-7 membered ring, which is optionally substituted with halogen, CN, C₁₋₄ alkyl optionally substituted with 1-3 F, C₁₋₄ alkoxy optionally substituted with 1-3 F, or C₁₋₄ heteroalkyl optionally substituted with 1-3 F, and any remaining R¹⁰¹ is defined in (i) or (ii).

21-23. (canceled)

24. The compound of claim 1, or a pharmaceutically acceptable salt thereof, characterized as having a structure according to Formula A-3:

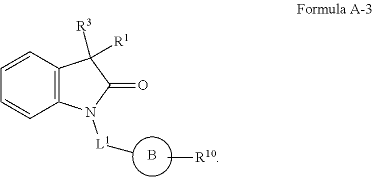

25. A compound selected from the following table or a pharmaceutically acceptable salt thereof.

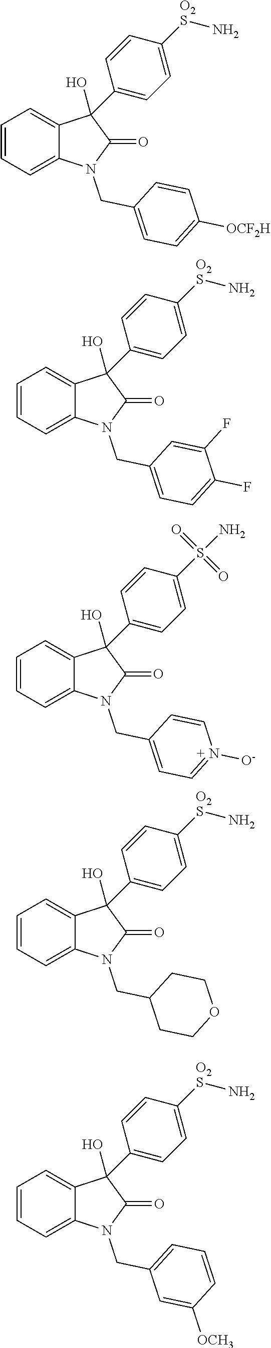

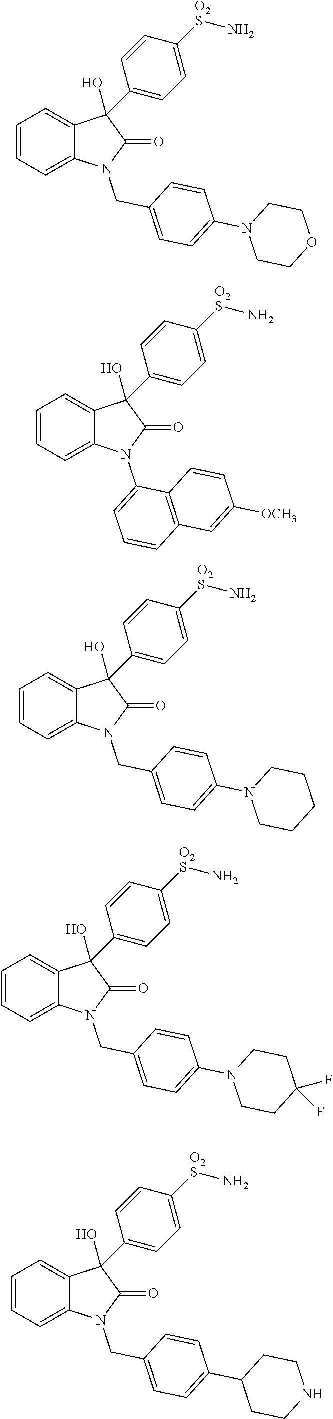 [SMILES: NS(=O)(=O)c1ccc(C2(O)C(=O)N(Cc3ccc(N4CCOCC4)cc3)c3ccccc32)cc1 ; COc1ccc2c(N3C(=O)C(O)(c4ccc(S(N)(=O)=O)cc4)c4ccccc43)cccc2c1 ; NS(=O)(=O)c1ccc(C2(O)C(=O)N(Cc3ccc(N4CCCCC4)cc3)c3ccccc32)cc1 ; NS(=O)(=O)c1ccc(C2(O)C(=O)N(Cc3ccc(N4CCC(F)(F)CC4)cc3)c3ccccc32)cc1 ; NS(=O)(=O)c1ccc(C2(O)C(=O)N(Cc3ccc(C4CCNCC4)cc3)c3ccccc32)cc1]

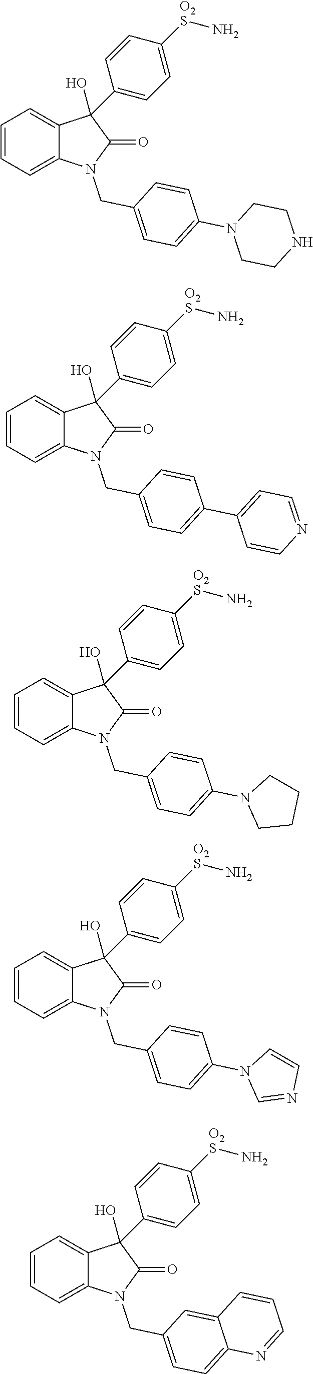 [SMILES: NS(=O)(=O)c1ccc(C2(O)C(=O)N(Cc3ccc(N4CCNCC4)cc3)c3ccccc32)cc1 ; NS(=O)(=O)c1ccc(C2(O)C(=O)N(Cc3ccc(-c4ccncc4)cc3)c3ccccc32)cc1 ; NS(=O)(=O)c1ccc(C2(O)C(=O)N(Cc3ccc(N4CCCC4)cc3)c3ccccc32)cc1 ; NS(=O)(=O)c1ccc(C2(O)C(=O)N(Cc3ccc(-n4ccnc4)cc3)c3ccccc32)cc1 ; NS(=O)(=O)c1ccc(C2(O)C(=O)N(Cc3ccc4ncccc4c3)c3ccccc32)cc1]

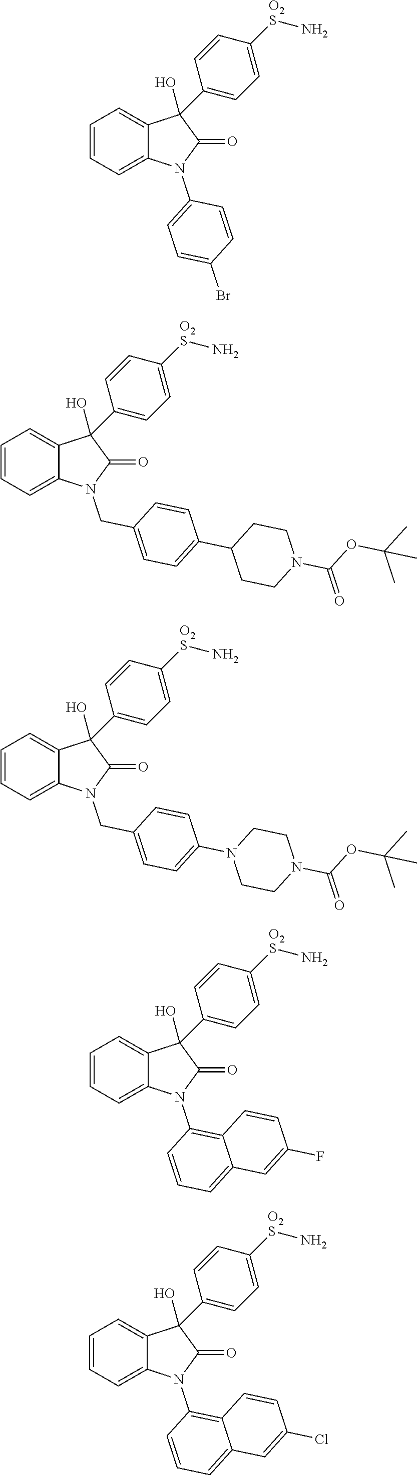 [SMILES: NS(=O)(=O)c1ccc(C2(O)C(=O)N(c3ccc(Br)cc3)c3ccccc32)cc1 ; CC(C)(C)OC(=O)N1CCC(c2ccc(CN3C(=O)C(O)(c4ccc(S(N)(=O)=O)cc4)c4ccccc43)cc2)CC1 ; CC(C)(C)OC(=O)N1CCN(c2ccc(CN3C(=O)C(O)(c4ccc(S(N)(=O)=O)cc4)c4ccccc43)cc2)CC1 ; NS(=O)(=O)c1ccc(C2(O)C(=O)N(c3cccc4cc(F)ccc34)c3ccccc32)cc1 ; NS(=O)(=O)c1ccc(C2(O)C(=O)N(c3cccc4cc(Cl)ccc34)c3ccccc32)cc1]

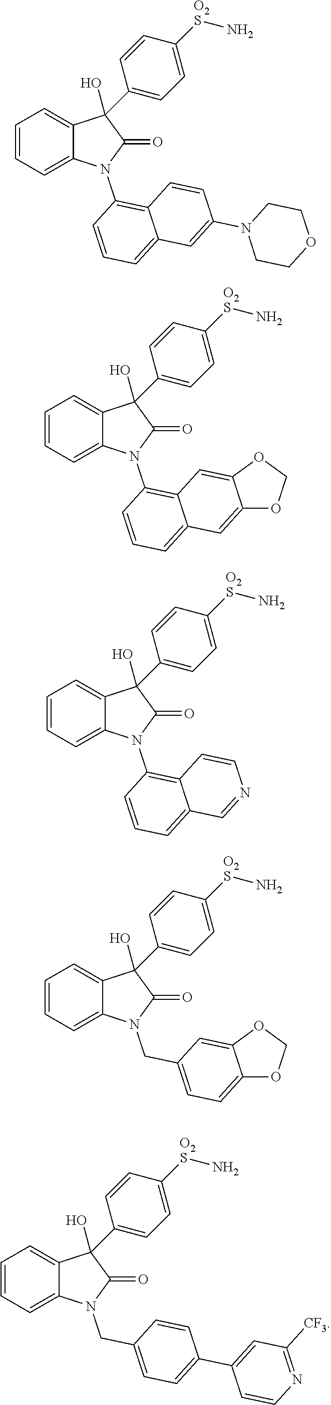 [SMILES: NS(=O)(=O)c1ccc(C2(O)C(=O)N(c3cccc4cc(N5CCOCC5)ccc34)c3ccccc32)cc1 ; NS(=O)(=O)c1ccc(C2(O)C(=O)N(c3cccc4cc5c(cc34)OCO5)c3ccccc32)cc1 ; NS(=O)(=O)c1ccc(C2(O)C(=O)N(c3cccc4cnccc34)c3ccccc32)cc1 ; NS(=O)(=O)c1ccc(C2(O)C(=O)N(Cc3ccc4c(c3)OCO4)c3ccccc32)cc1 ; NS(=O)(=O)c1ccc(C2(O)C(=O)N(Cc3ccc(-c4ccnc(C(F)(F)F)c4)cc3)c3ccccc32)cc1]

26. The compound: 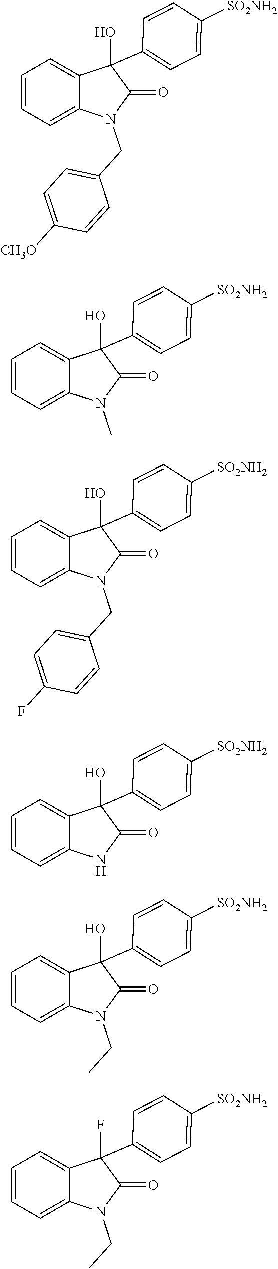 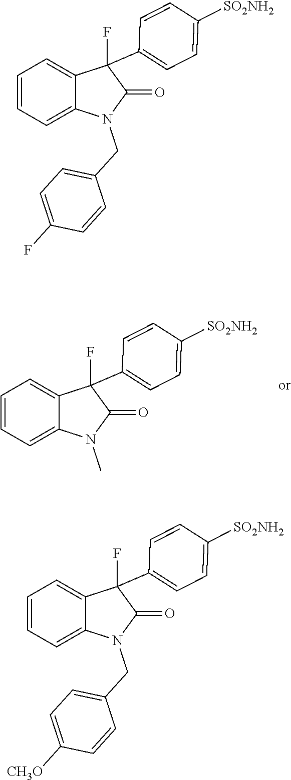
or a pharmaceutically acceptable salt thereof.

27. A pharmaceutical composition comprising a compound as described in claim 1 or a pharmaceutically acceptable salt thereof and a pharmaceutically acceptable excipient.

28. A method of inhibiting a carbonic anhydrase enzyme in vitro or in vivo comprising contacting the carbonic anhydrase enzyme with an effective amount of a compound as described in claim 1 or a pharmaceutically acceptable salt thereof.

29. A method of treating a disease or condition mediated by a carbonic anhydrase enzyme in a mammal (e.g., a human), comprising administering a compound as described in claim 1, or a pharmaceutically acceptable salt thereof, to the mammal.

30-34. (canceled)

35. The method of claim 29, wherein the disease or condition mediated by a carbonic anhydrase enzyme is an allergic disease, a bacterial infection, a fungal infection, a viral infection, mastocytosis or mast cell-mediated inflammation.

36-44. (canceled)
